(19) United States
(12) Patent Application Publication
(10) Pub. No.: US 2020/0303074 A1
(43) Pub. Date: Sep. 24, 2020

(54) INDIVIDUALIZED AND COLLABORATIVE HEALTH CARE SYSTEM, METHOD AND COMPUTER PROGRAM

(71) Applicant: Martin Mueller-Wolf, Zug (CH)

(72) Inventor: Martin Mueller-Wolf, Zug (CH)

(21) Appl. No.: 16/841,510

(22) Filed: Apr. 6, 2020

Related U.S. Application Data
(63) Continuation-in-part of application No. 14/373,575, filed on Jul. 21, 2014, now Pat. No. 10,692,589.

(51) Int. Cl. G16H 50/20; G16H 10/60; G16H 50/50; G16H 40/67; G16H 70/20; G16H 70/60; G06Q 50/00; G16H 20/70; G16H 80/00; G16H 10/20; G16H 20/30; A61B 5/00; A61B 5/16; A61B 5/021; A61B 5/145; H04N 7/14

(52) U.S. Cl. CPC G16H 50/20; H04N 7/141; G16H 50/50; G16H 40/67; G16H 70/20; G16H 70/60; G06Q 50/01; G16H 20/70; G16H 80/00; G16H 10/20; G16H 20/30; A61B 5/486; A61B 5/165; A61B 5/4833; A61B 5/7275; A61B 5/7465; A61B 5/7475; A61B 5/021; A61B 5/14546; A61B 5/14532; G16H 10/60

(57) ABSTRACT

A system and method for individualized life management focusing on individualized and collaborative health care involving a plurality of individuals, using groups of state parameters for defining a state of each individual, and using groups of action parameters for defining treatment options and/or behavior options targeted at an individual. The system includes a data processor for processing input data, based on the groups of state parameters, into output data, which are the basis for the groups of action parameters, using defined relationships/assignments between groups of state parameters and groups of action parameters. Data storage stores the groups of state parameters and action parameters and the defined relationships/assignments between groups of the state and action parameters. A data communication system/platform communicates state parameters and/or action parameters among the individuals. The data processor means can include an adaptive structure (e.g., neural networks) where the defined relationships/assignments between groups are redefined/updated using empirical pairs of action parameter groups and state parameter groups.

### CROSS-REFERENCE TO RELATED APPLICATION

This application is a continuation-in-part under 35 U.S.C. § 120 based upon co-pending U.S. patent application Ser. No. 14/373,575 filed on Jul. 21, 2014, which is a national stage entry under 35 U.S.C. § 371 based upon International Application No. PCT/IB2013/000183 filed on Jan. 20, 2013, which claims priority under 35 U.S.C. § 119(e) based upon U.S. provisional patent application Ser. No. 61/588,721 filed on Jan. 20, 2012 and U.S. provisional patent application Ser. No. 61/752,887 filed on Jan. 15, 2013. The entire disclosure of the prior applications are incorporated herein by reference.

### COPYRIGHT AUTHORIZATION

A portion of the disclosure of this patent document contains material which is subject to (copyright or mask work) protection. The (copyright or mask work) owner has no objection to the facsimile reproduction by anyone of the patent document or the patent disclosure, as it appears in the Patent and Trademark Office patent file or records, but otherwise reserves all (copyright or mask work) rights whatsoever.

### BACKGROUND

### Technical Field

The present technology relates to a system and method for individualized life management in connection with individualized and collaborative health care involving a plurality of individuals, using groups of state parameters for defining a state of each individual, and using groups of action parameters for defining treatment options and/or behavior options targeted at an individual.

### Background Description

In known healthcare systems and methods, there is a focus on health management instead of disease management. Some 3000 years ago, in China, health management was literally health-oriented. It was therefore comprehensive and integrative and therefore necessarily preventive. The ancient Chinese Health Care Professionals (HCP's) were rewarded for the health of their clients, and not for treating diseases.

On the contrary, today's health care systems can be defined as health reparation systems or disease management systems for which it is a common saying in the American Medical Community that there are rushed doctors working in a fragmented system.

It is known that there exists alienation from individual health management, resulting in a fragmented health care system

It seems that the citizens of First World countries in the so-called “Trias” (North America; Europe; Japan plus ASEAN) are separated from their health.

Especially in the United States of America (USA), the pharmaceutical companies and the payors, the insurance companies, are more or less in one hand. Thus, the doctor working in a health maintenance organization (HMO) is very much in a situation of an economically dependent person (and economic victim) with the patient so to say being the victim of the victim.

The USA makes up 4% of the First World population. They spend 40%, resulting in a factor of 10, for disease management with very poor results: 66% of the population are overweight, 34% are obese, and the rate of diabetic's type 2 (which is a result of the individual health management of the persons concerned) is by far the highest in all First World countries. This situation has been described by Prof. Dr. Paul Ciechanowski, a leading US expert for Diabetes Management, Depression Management, “Diapression” (“An Integrated Model for Understanding the Experience of Individuals With Co-Occurring Diabetes and Depression”, 2011): “the rushed doctor in a fragmented system”.

Therefore, a comprehensive and integrative person/patient-centered health care model is needed. Health education is not dealt with in elementary, secondary or high schools—nor in colleges or at universities. Although it is the most valuable good of mankind, it is not treated and protected as such.

As an example, the role concepts of patients and doctors in the western world for the USA and German/Europe, will be discussed.

The research in Europe (in Germany) which also reflects results in the USA and Japan (although the frequency in the groups is certainly different in these countries and the social background influences the results so that in each country a specific analysis is needed) is described in the following in order to give some basic insight.

The following pattern of patients exist:
Group 1: DETERMINISTIC GROUP: Health is determined by fate (good or back luck).
Group 2: MEDICAL BELIEVER GROUP: I cannot do anything. My (high quality) doctor is in charge of my health.
Group 3: NATURE GROUP: Avoid the doctor and the medical institutions. Live healthy—and everything will be fine.
Group 4: ENLIGHTENED COLLABORATIVE CARE GROUP: I am aware of the fact that it is my health and my life: So I am looking for a doctor/HCP as a professional partner and act as a more or less self-conscious and responsible partner of my doctor and/or the health care professionals.

The doctors have corresponding role concepts:
Authoritarian doctors like the deterministic group patients. These patients listen to the doctor as if he was fate or even God.
The paternalistic doctors prefer the medical believer group. They are seen as an authority and the patients cling to their lips.

All groups of doctors are somewhat distant and skeptical about the nature group, which avoids contact with the doctors and is more of an anti-business model. The enlightened collaborative care group is officially preferred by all doctors. But one thing is what is said in theory (We all like and strive for collaborative care), the reality may be far away from it. According to several research results, 80% of the patients in the USA receive about 20% of the health care visit time of the American doctors. The other 20%, the “system-preferred” receive 80% of the health care visit time.

In known standardized medical treatment, it is evident and need not be proved that first of all, standardized medical care is necessary for all patients to create a basis (basic service).

In some cases, the patient as an object vs. the responsible empowered self-conscious patient. Again, there is no need to argue that the patient as an object certainly receives the minimum care and has good chances to survive.

For an optimum life span, for best quality of life, and for a best medical treatment in the case of illness, however, clearly the empowered patient, showing initiative, empowerment and being able to carry out a high quality self-care has the better life.

It can be appreciated that one basis for collaborative care includes openness, trust, and a positive doctor-patient relationship. This again is obvious and need not be proved (although there is a huge amount of research data proving this as an empirical fact).

Medical care has improved enormously in the last century. The life expectancy of today's generations has been increased significantly. Where, however, addictive patterns and very change-resisting behavior patterns are prevalent, the classical care situation with a short contact between patient and doctor reaches its limits.

This is true for all chronic diseases. So there is a need for the patients with chronic diseases to receive treatment support or even adaptation and behavior modification support.

It can be appreciated that there is a need for lifelong support for chronical disease patients utilizing an Individualized Support Management (ISM). All the existing research has shown that patients with chronic diseases need support and there are altogether four sources:
(1) the person himself/herself (self-motivation, internet contacts, health care education, training etc.);
(2) the direct social environment (support by partner, family, and friends);
(3) the “second” social environment and groups (like patient support groups, training groups, and self-care groups);
(4) the medical support by doctors and health care practitioners (as the last—and financially most expensive and also limited—resource).

There of course are corresponding challenges and solutions for the existing problems.

One such is standardized treatment. The health care repair systems of today (with the rushed doctor in a fragmented system) are disease-focused with patients as (more or less) an object of a (more or less) standardized treatment.

Another such is separation from the own health. The modern patients are more or less separated from or alienated by their own health; only very few (less than 10% of the population) are really fully empowered and in charge of their individual health management.

Still another such is a need for help. Both, patients and doctors, need help.

Let us take the example of the US American society: More than 50% of the doctors suffer from burnout syndrome and doctors starting show the normal depression rate of the population (4%) which increases after one year up to striking 25%.

Let us take the following examples of diabetes care: Only 7% of the US patients reach the three objectives which are relevant to preserve their lives: reaching the blood pressure goals, reaching the objectives for lipids/cholesterol, and reaching the average level HbA_{1c} for blood sugar, avoiding extreme hypoglycemic and hyperglycemic states.

In the exemplary, all diabetes type 2 patients are certainly checked in terms of bio-medical status (level 1). This is, however, only the peak of the iceberg.

If the patient is treated as an object in a standardized treatment procedure, the results are inferior (especially in person- and psychology-related chronic diseases).

### Example 1

More than 30% of diabetes patients with strong depression (about 12%) and some 20% with clear depressive tendencies (Paul Ciechanowski, MD, PHD, article on “Diapression”: “Diapression: An Integrated Model for Understanding the Experience of Individuals With Co-Occurring Diabetes and Depression”, 2011) are not reached. It is evident that a person suffering from depression is not open for a high quality self-care diabetes treatment.

### Example 2

Some 50-70% of the patients with diabetes mellitus type 2 suffer from an “eating addiction” (F. Kiefer, M. Grosshans, “Beitrag der Suchtforschung zum Verstandnis der Adipositas”, 2009), and show the same symptoms/activity patterns in their brain when looking at their favorite “juicy hamburger” or other favorite food as alcoholics do when looking at alcohol.

### Example 3

It is also evident that Adipositas Patients who are eating addicts in diabetes mellitus type 2 need support and a psychiatric treatment (Prof. F. Kiefer, University of Heidelberg, Central Institute for Addictive Diseases, Mannheim) and that a normal rational appeal will help as little as telling a heroin or alcohol addict: “It would be better if you did not take heroin or if you did not drink alcohol.”

Regarding the patient as an Object within a highly complex technological process, the cost-driven medical care and health care systems of today have the effect that the patients have become more and more an object within a highly complex technological process. The very disappointing results with chronic diseases and with all diseases, which need to take into account the needs of the person, show that there is a definite need for change.

There is a threshold and barrier between many patients and doctors, which needs to be overcome. This, however, is very difficult especially for the complex topics and needs of treating chronic diseases and treating diseases with intimate personal aspects, which require to understand the psychology and the personal situation of a patient in order to empower him to be a client.

Lifestyle adaption and behavior modification for diabetes type 2 patients as well as for patients with depression or the combination of both, patients with depression as well as support for patients with diabetes type 1 (psychological treatment support) is not achieved by rational appeals or logic.

All patients with chronic diseases facing (for depressive patients twice in a lifespan) a crisis where they need definite and urgent support. Leaving patients with chronic diseases alone for themselves does not lead to best results.

It can be appreciated that a need exists for a new and novel individualized and collaborative health care system, method and computer program that can be used for the creation of an Individualized Action Programs (IAP) with an Individualized Self-Care (ISC), with an Individualized Support Program (ISP) and with an Individualized Treatment Scheme (ITS) for the present technology. In this regard, the present technology substantially fulfills this need. In this respect, the individualized and collaborative health care system, method and computer program according to the present technology substantially departs from the conventional concepts and designs of the known systems and methods, and in doing so provides an apparatus and/or method primarily developed for the purpose of creating the IAP with the ISC, with the ISP and with the ITS.

### SUMMARY

In view of the foregoing disadvantages inherent in the known types of health care management systems and methods now present, the present technology provides a novel individualized and collaborative health care system, method and computer program, and overcomes one or more of the mentioned disadvantages and drawbacks of the prior art. As such, the general purpose of the present technology, which will be described subsequently in greater detail, is to provide a new and novel individualized and collaborative health care system, method and computer program and method which has all the advantages of the prior art mentioned heretofore and many novel features that result in an individualized and collaborative health care system, method and computer program which is not anticipated, rendered obvious, suggested, or even implied by the prior art, either alone or in any combination thereof.

According to one aspect, the present technology can include a system for individualized and collaborative health care using groups of state parameters for defining a state of each individual, and using groups of action parameters for defining treatment options, support options and/or behavior options targeted at an individual within said plurality of individuals. The system can include at least one sensor configured to ascertain physiological or psychological sensor data of the targeted individual. A computer system in communication with the sensor, the computer system comprises one or more processors programmed with computer program instructions which, when executed cause the computer system to convert the sensor data to a sensor data vector in a defined sequence. To process input data that is based on the groups of state parameters and the sensor data vector, into output data, which are the basis for the groups of action parameters, using defined relationships/assignments between groups of state parameters and groups of action parameters. To process one or more estimators based on the sensor data vector in a hierarchical manner. To store, on at least one data storage device, the groups of state parameters, the groups of action parameters and the defined relationships/assignments between groups of state parameters and groups of action parameters. To define at least one state of each of the individuals using the output data, the state of the individuals being in part defined from a social module, a personal module and a psychological module that are implemented by the computer system. To receive medical information about the individuals. To compare the state of the individuals and the medical information by determining a deviation from at least part of the state of the individuals and at least part of the medical information. To define at least one treatment or behavior option using the groups of action parameters, the action parameters being defined in part from the social module, the personal module, the psychological module, and the deviation. To target the treatment or behavior option to a targeted individual within the plurality of individuals. To generate a predicted state of health of the targeted individual at a pre-determined time period utilizing a neural chain of the estimators, and classifying the targeted individual to a category of a plurality of categories according to the predicted state, and providing the predicted state of health with the treatment or behavior option. To communicate to the targeted individual, by way of a data communication system, the treatment or behavior option, state parameters selected from the groups of state parameters and/or action parameters selected from the groups of action parameters among the plurality of individuals. The system can further include a graphical user interface operably implemented or implementable on the computer system and executable by the processors.

According to another aspect, the present technology can include a method for individualized and collaborative health care involving a plurality of individuals, using groups of state parameters that define a state of each individual, and using groups of action parameters that define individualized treatment options, individualized support options and/or individualized behavior options targeted at a targeted individual within the plurality of individuals, the method being implemented in a computer system that includes one or more physical processors configured to execute one or more computer program modules. The method can include ascertaining physiological or psycho-medical sensor data of the targeted individual utilizing at least one sensor. Converting, using the processors, the sensor data to a sensor data vector in a defined sequence. Processing, using the processors of the computer system, input data received by the computer system and the sensor data vector, which are based on the groups of state parameters, into output data, which are the basis for the groups of action parameters, using defined relationships/assignments between groups of state parameters and groups of action parameters. Storing, on at least one data storage device of the computer system, the groups of state parameters, the groups of action parameters and the defined relationships/assignments between groups of state parameters and groups of action parameters. Defining, using the processors of the computer system, at least one state of each of the individuals using the output data, the state of the individuals being in part defined from a social module, a personal module and a psychological module. Processing, using the processors of the computer system, medical information associated with the individuals. Comparing, using the processors, the state of the individuals and the medical information by determining a deviation from at least part of the state of the individuals and at least part of the medical information. Defining, using the processors of the computer system, at least one treatment or behavior option or an individualized action program using the groups of action parameters, the action parameters being defined in part from the social module, the personal module, the psychological module, and the deviation. Processing, using the processors of the computer system, one or more estimators based on the sensor data vector in a hierarchical manner. Generating a predicted state of health of the targeted individual at a pre-determined time period utilizing a neural chain of the estimators, and classifying the targeted individual to a category of a plurality of categories according to said predicted state, and providing the predicted state of health with the treatment or behavior option or the individualized action program. Communicating to the targeted individual the treatment or behavior option using a communication interface of the computer system, state parameters selected from the groups of state parameters and/or action parameters selected from the groups of action parameters among the plurality of individuals. Initiating direct communication between the targeted individual and a health care professional by way of a graphical user interface operably implemented or implementable on the computer system and executable by the processors.

According to yet another aspect, the present technology can include a non-transitory computer readable medium with an executable program stored thereon comprising instructions for execution by at least one processing unit for individualized and collaborative health care involving a plurality of individuals, using groups of state parameters that define a state of each individual, and using groups of action parameters that define individualized treatment options, individualized support options and/or individualized behavior options targeted at a targeted individual within the plurality of individuals, such that the instructions when executed by the at least one processing unit cause the at least one processing unit to ascertain physiological or psycho-medical sensor data of the targeted individual utilizing at least one sensor. To convert, using the processors, the sensor data to a sensor data vector in a defined sequence. To process, using the processors of the computer system, input data received by the computer system and the sensor data vector, which are based on the groups of state parameters, into output data, which are the basis for the groups of action parameters, using defined relationships/assignments between groups of state parameters and groups of action parameters. To store, on at least one data storage device of the computer system, the groups of state parameters, the groups of action parameters and the defined relationships/assignments between groups of state parameters and groups of action parameters. To define, using the processors of the computer system, at least one state of each of the individuals using the output data, the state of the individuals being in part defined from a social module, a personal module and a psychological module. To process, using the processors of the computer system, medical information associated with the individuals. To compare, using the processors, the state of the individuals and the medical information by determining a deviation from at least part of the state of the individuals and at least part of the medical information. To define, using the processors of the computer system, at least one treatment or behavior option or an individualized action program using the groups of action parameters, the action parameters being defined in part from the social module, the personal module, the psychological module, and the deviation. To process, using the processors of the computer system, one or more estimators based on the sensor data vector in a hierarchical manner. To generate a predicted state of health of the targeted individual at a pre-determined time period utilizing a neural chain of the estimators, and classifying the targeted individual to a category of a plurality of categories according to the predicted state, and providing the predicted state of health with the treatment or behavior option or the individualized action program. To communicate to the targeted individual the treatment or behavior option using a communication interface of the computer system, state parameters selected from the groups of state parameters and/or action parameters selected from the groups of action parameters among the plurality of individuals. To initiate direct communication between the targeted individual and a health care professional by way of a graphical user interface operably implemented or implementable on the computer system and executable by the processors.

In some embodiments, the graphical user interface can be configured or configurable to initiate direct communication between the targeted individual and a health care professional.

In some embodiments, the direct communication can be video chat utilizing a camera in operable communication with the processor.

In some embodiments, the state parameter group can be based on observation, evaluation and assessment of the health care client using a web-based questionnaire sent to the targeted individual by way of a communication interface of the computer system.

In some embodiments, the web-based questionnaire can be configured or configurable to provide information regarding self-assessments of a medical and physiological condition of the targeted individual, information regarding a psychological condition of the targeted individual, information regarding a personality trait, communication style, genetic factors, and/or behavior patterns of the targeted individual, and information regarding fitness, activities, and/or lifestyle of the targeted individual.

In some embodiments, the information from the web-based questionnaire can be used in part by the processor to define at least one parameter in the group of state parameters by assigning a marker or value for the targeted individual, and wherein the client-specific action parameter group is created by the processor where each parameter in the client-specific action parameter group is assigned a marker or value for the targeted individual.

In some embodiments, the treatment or behavior option can include at least one report selected from the group consisting of rating the targeted individual condition associated with groups of success factors relating to at least one question in the web-based questionnaire, supporting further detailed self-assessment of the targeted individual, and categorizing an action to be conducted by the targeted individual.

In some embodiments, the treatment or behavior option can include need-for-action levels selected from the group consisting of a first level where the deviation is determined to be at a first predetermined value, a second level where the deviation is determined to be at a second predetermined value that is less than the first predetermined value, a third level where the deviation is determined to be a third predetermined value that is less than the second predetermined value, and a fourth level where no deviation is found.

In some embodiments, the computer system can include a data interface for data acquisition. The data interface can be configured or configurable to receive biomedical information selected from one or any combination of blood pressure, lipids, and blood glucose level.

In some embodiments, the defined relationships/assignments between groups can be redefined/updated using empirical pairs/empirically defined relations and neural networks determined relations of action parameter groups and state parameter groups. The neural networks can comprise a self-organizing map constructed from a set of the action parameters, a set of predetermined action levels, and corresponding predetermined disease progression data.

In some embodiments, the estimators can be coded to be placed on a topologically closed, two-dimensional surface on a regular or irregular grid formed of the estimators configured to assign a same number of adjacent estimators to every the estimator.

Some embodiments of the present technology can further include the step of defining at least one parameter in the group of state parameters by in part using the information from the web-based questionnaire to assign a marker or value for the targeted individual.

Some embodiments of the present technology can further include the step of creating the client-specific action parameter group where each parameter in the client-specific action parameter group is assigned a marker or value for the targeted individual.

Some embodiments of the present technology can further include the step of creating at least one report and associating the report with the treatment or behavior option. The report can be one or any combination of rating the targeted individual condition associated with groups of success factors relating to at least one question in the web-based questionnaire, supporting further detailed self-assessment of the targeted individual, and categorizing an action to be conducted by the targeted individual.

Some embodiments of the present technology can further include the step of receiving biomedical information using a data interface of the computer system. The data interface can be configured or configurable for data acquisition. The biomedical information can be one or any combination of blood pressure, lipids, and blood glucose level, and wherein the treatment or behavior option is at least in part dependent on the biomedical information.

Another exemplary aspect of the present technology can pertain to a system for individualized and collaborative health care involving a plurality of individuals, using groups of state parameters for defining a state of each individual, and using groups of action parameters for defining treatment options, support options and/or behavior options targeted at an individual within the plurality of individuals. The system can include a data processor means adapted for processing input data, which are based on the groups of state parameters, into output data, which are the basis for the groups of action parameters, using defined relationships/assignments between groups of state parameters and groups of action parameters. A data storage means can be adapted for storing the groups of state parameters, the groups of action parameters and the defined relationships/assignments between groups of state parameters and groups of action parameters. A data communication system/platform can be adapted for communicating state parameters selected from the groups of state parameters and/or action parameters selected from the groups of action parameters among the plurality of individuals.

The data processor means can comprise an adaptive structure where the defined relationships/assignments between groups are redefined/updated using empirical or by neural network analysis defined relations and correspondences pairs of action parameter groups and state parameter groups. The adaptive structure can include one or any combination of expert systems, fuzzy logic, neural networks, genetic and/or evolutionary algorithms and combinations thereof. The system can be web-based including one or more of PC-application, tablet application, iPhone and smartphone-technology and other electronic communication devices. The groups of state parameters can include one or any combination of biomedical/physiological (B), psychological (P), personal (P) and socio-economic (S) characteristics/attributes of health care clients. A health care client-specific state parameter group can be based on assessment of the health care client using a questionnaire for the self-assessment.

Still another exemplary aspect of the present technology can pertain to a method for individualized and collaborative health care involving a plurality of individuals, using groups of state parameters that define a state of each individual, and using groups of action parameters that define individualized treatment options, individualized support options and/or individualized behavior options targeted at an individual within the plurality of individuals. The method can include processing input data, which are based on the groups of state parameters, into output data, which are the basis for the groups of action parameters, using defined relationships/assignments between groups of state parameters and groups of action parameters. The groups of state parameters, the groups of action parameters and the defined relationships/assignments between groups of state parameters and groups of action parameters are stored. State parameters selected from the groups of state parameters and/or action parameters selected from the groups of action parameters are communicated among the plurality of individuals.

The defined relationships/assignments between groups can be redefined/updated using empirical pairs/empirically defined relations and neural networks determined relations of action parameter groups and state parameter groups. The groups of state parameters can include one or any combination of biomedical/physiological, psychological, personal and socio-economic characteristics/attributes of health care clients. A health care client-specific state parameter group can be determined by assessing the health care client using a questionnaire. The health care client-specific state parameter group can be repeatedly determined throughout the health care client's affiliation to the plurality of individuals. Communication and information exchange can be made available: among individuals belonging to a first subset (HCC), and family, friends, social environment of the plurality of individuals; among individuals belonging to a second subset (HCP) of the plurality of individuals; and between individuals belonging to the first subset (HCC) and individuals belonging to the second subset (HCP). Defined relationships/assignments between action parameter groups and state parameter groups can be made available for communication and information exchange among the plurality of individuals. The individuals of the plurality of individuals can be categorized into different categories of individuals based on their respective state parameter groups and corresponding action parameter groups.

Yet another exemplary aspect A of the present technology can pertain to a method for individualized and collaborative health care involving a plurality of individuals, including providing state parameters for defining a state of each individual health care client (HCC), wherein the state parameters are based on information including one or any combination of biomedical, physiological, psychological, personal and socio-economic information about the HCC and combinations thereof. The HCC, who has a health management task, conducts a self-assessment based on the state parameters. A report of the self-assessment of the HCC is provided to a health care professional (HCP). The HCP has physiological and biomedical tests conducted on the HCC and has biomedical facts obtained from the HCC concerning the health management task, which can include facts as to health development, an individualized prevention program, self-care, and individualized support. Need-for-action levels are determined indicating urgency in need for action in addressing the health management task (e.g., treating the disease or the health problem) of the HCC. The need-for-action levels are determined by comparing an extent of a deviation between results of the self-assessment compared to results of the physiological and biomedical tests and the biomedical facts, and thereby evaluating the HCC's risks and chances. The HCP conveys to the HCC the need-for-action levels, thereby providing the HCC with a learning model in self-care and individualized disease management. The HCP uses the need-for-action levels to determine appropriate action parameters including an individualized and collaborative health care action plan (“Individualized Action Program”) for the HCC.

The defined relationships/assignments between the action parameters and the state parameters can be made available for communication and information exchange among the plurality of individuals. The need-for-action levels can include one or any combination of a first level where the deviation is determined to be extreme, a second level where the deviation is determined to be definite, a third level where the deviation is determined to be some difference, and a fourth level where no deviation is found. The action parameters can include medical therapy groups and/or prevention or support groups of the HCCs and others exchanging information about the health management task (e.g., disease or the health problem) or a life management task (see ILM task), which medications, the support groups and the medical therapies have been successful, partially successful or were a failure or create experience-based options that can be used for the Individualized Action Program. The self-assessment can be a web-based questionnaire, which is sent to the HCC via a communication network linking places or things including one or any combination of individual hospitals, health care professionals' practices or clinics, offices of support groups, the HCCs homes, mobile wireless communication devices of the HCCs and the HCPs, and combinations thereof. The prevention or support program or the health management task (e.g., treating or preventing a disease or health problem) can pertain to one or more of cardiovascular disease, diabetes, depression, alcoholism, obesity, overweight, stress, burn-out, psychosomatic disease, gastro-intestinal disease, chronic orthopedic disease, chronic pain-related disease, any other chronic disease, drug addiction and combinations thereof. The health management task can be treating or preventing diabetes and the Individualized Action Program for the HCC is tailored to reaching blood pressure goals, reaching objectives for lipids/cholesterol, and reaching an average level HbA_{1c} for blood sugar, avoiding extreme hypoglycemic and hyperglycemic states. The health management task can comprise treating or preventing diabetes and the physiological tests pertain to one or more of blood glucose or HbA_{1c} level, lipid level and cholesterol level of the HCC and measurements of one or both of weight and blood pressure can be used with the physiological tests.

Still yet another exemplary aspect B of the present technology can include all of the features of the third aspect A and can include utilizing at least the need-for-action levels and the action parameters in connection with a neural network system to determine a prediction of future development of the disease or the health problem.

The neural network can further source the groups of state parameters defined in the state of each individual to determine the prediction of future development of the disease or health problem, a related cost of disease management or health and prevention. The neural network can further source a validated database of pairs of groups of state parameters and groups of action parameters. This method can include the step of iteratively conducting the HCC's self-assessment and the HCP's obtaining of the physiological and biomedical tests, to update the need-for-action levels and the action parameters. This method can include the step of iteratively determining the prediction of future development of the disease or health problem, management, related cost or health and prevention, based on the updating of the need-for-action levels and the action parameters.

A self-organizing map is a type of neural network that can produce low-dimensional representations of an input space, which typically comprises high-dimensional data. The self-organizing map is self-learning in that the network is built via unsupervised learning (i.e., an unknown structure is derived from unlabeled data) and, thus, particularly useful in situations where a relationship between input and output is not fully known. The self-organizing map is also capable of preserving topological properties of the input space.

Regarding another specific feature of the aspect B or any other neural network aspect described herein, the neural network system comprises a self-organizing map constructed, via unsupervised learning, from a set of predetermined action parameters, a set of predetermined action levels, and corresponding predetermined disease progression data.

Another exemplary aspect of the present technology can feature a method for individualized and collaborative action planning for an individual, including obtaining information, from the individual and a professional responsible to the individual, related to a set of parameters, the set of parameters including one or more of biomedical and physiological parameters, psychological parameters, personality parameters, and socio-economic parameters. Another step is determining, for each of a plurality of success factors, a priority indicating a level of urgency of action in order to achieve the success factor. Another step is classifying the individual, utilizing a neural network structure, into a group among a set of groups based on priorities respectively determined for the plurality of success factors. Yet another step is determining an individualized and collaborative action plan for the individual (“Individualized Action Program”) based on the group to which the individual is classified.

Referring to specific features of this aspect, the priority can include one or any combination of no need for action, some need for action, definite need for action, and urgent need for action. The Individualized Action Program can include option for actions to be undertaken by at least one of the individual or the professional to achieve the plurality of success factors. The method can include the step wherein at least one of the options for action and the Individualized Action Program to be undertaken by the individual or the professional relate to a condition selected from the group consisting of a health condition, financial condition, socialization condition, political condition, economical condition, and life culture condition of the individual, and combinations thereof.

Still further another exemplary aspect of the present technology can feature an apparatus including a processor coupled to a memory, the processor being configured to:
generate a model of a state of an individual relative to a condition of the individual;
generate, utilizing a set of neural networks, a predicted state of the individual at a pre-determined time period and classify the individual to a category of a plurality of categories according to the predicted state; and
determine a customized action, based on the predicted state and the category associated with the individual, for the individual to perform to align an actual state at the pre-determined time period with a pre-determined goal state, thereby creating a controlling system for “Individualized Action Programs” based on predictions, goals and comparisons with achievements.

Referring to specific features of the this aspect, the condition of the individual can include one or more of (e.g., is selected from the group consisting of) a health condition, self-care condition, support condition, treatment adherence condition, financial condition, socialization condition, political condition, economical condition and life culture condition, of the individual, and combinations thereof. The health condition can include one or any combination of: cardiovascular disease, diabetes, depression, alcoholism, obesity, overweight, stress, burn-out, psychosomatic disease, gastro-intestinal disease, chronic orthopedic disease, chronic pain-related disease, any other chronic disease, drug addiction and combinations thereof. The model of the state of the individual can include a set of parameters that includes at least one of a biomedical or physiological-based parameter, a communication style or psychological-based parameter, a personality-based parameter, and a socio-economical-based parameter. The set of parameters can be at least partially derived from one or more questionnaires completed by the individual. The category to which the individual is classified can indicate a level of risk and expected costs for health management to which the individual is exposed according to the predicted state. The apparatus can be integrated in a blood glucose monitor or programmed on a blood glucose meter, PC, tablet, iPhone or smart phone device.

Yet even another exemplary aspect of the present technology can feature a method for treating diabetes, including generating a patient model that specifies individual physiological and psychological parameters of a patient. The patient model is applied to a neural network system to determine a predicted state of blood pressure, cholesterol, lipids and blood glucose levels for the patient at a pre-determined time in the future. An Individualized Action Program is determined based at least in part on the physiological parameters of the patient model, to control blood pressure, cholesterol, lipids and blood glucose levels of the patient to a desired state or value within a pre-determined time interval or series of time intervals.

Referring to specific features of this aspect, the Individualized Action Program can include at least one of healthy eating, physical activity, support groups, medical therapy groups, insulin dose, no smoking, treatment of depression and treatment of eating addiction. The method can include utilizing a classifier to assign the patient to a category corresponding to a specific level of risk for blood pressure or weight problems, cholesterol or lipid risks or severe hyperglycemic or hypoglycemia. The level of risk can be associated with at least one of a short-term or long-term risk. The method can include utilizing the predicted state to establish a target function for an optimization system such as the Individualized Action Program, implementable by a computer. The method can include utilizing a measure of effectiveness of the Individualized Action Program to establish the target function. The method can include the step of continuously updating the patient model based on psychological data and bio-medical and physiological data and treatment adherence as well as self-care behavior of the patient over time.

Another specific feature of this aspect can include the step of which determining the Individualized Action Program comprises applying the predicted state at the pre-determined time in the future to a second neural network system predicting and measuring validation criteria that can include at least one of health, success of disease management and costs of disease management over time.

Another exemplary aspect of the present technology can feature a method of using a computer system including a server and a database for individualized and collaborative action planning for treating a patient, including registering a patient into the system by inputting patient contact information into the system. The patient contact information is stored in the database. The server transmits a patient questionnaire to a remote device of the patient over a communication network. The server receives patient answers to questions on the patient questionnaire from the remote device of the patient. The patient answers include:
information regarding a patient self-assessment of the patient's medical and physiological condition,
information regarding the patient's psychological condition,
information regarding the patient's personality traits, genetic factors, and/or behavior patterns, and
information regarding a patient's fitness, activities, and/or lifestyle; storing the patient answers in the database.

The method can include the step of inputting medical information about the patient from a doctor treating the patient into the server for storing in the database. The computer system processes an assessment of the patient by comparing information from the patient answers and the medical information. The computer system automatically generates a report including action parameters for treating the patient.

Specific features of this aspect can include the following. The following step can be included in the method: wherein the step of comparing the information from patient answers to the medical information includes the step of determining a deviation from the information regarding the patient self-assessment of the patient's medical and physiological condition and the medical information input from the doctor. The method can also include the step wherein the report includes need-for-action levels indicating an urgency in a need for action based on the determined deviation. Another step can be used wherein the need-for-action levels include a first level where the deviation is determined to be extreme, a second level where the deviation is determined to be definite, a third level where the deviation is determined to be some difference, and a fourth level where no deviation is found. The database can include assessments of a plurality of other patients, and wherein the step of processing the assessment of the patient utilizes the assessments of the plurality of other patients. The medical information input by the doctor can include information derived from a review of the answers of the patient. The patient can access one or both of the assessment of the patient and the report.

Still another exemplary aspect of the present technology can feature a method of using a computer system including a server and a database for individualized and collaborative action planning for a particular patient, including registering a plurality of patients into the system by inputting patient contact information into the system. The patient contact information is stored in the database. The server transmits patient questionnaires to remote devices of the patients over one or more communication networks. The server receives patient answers to questions on the patient questionnaires from the remote devices of each of the patients. The patient answers are stored in the database. Also included is the step of storing medical information about the patients from one or more doctors treating the patients into the database. The computer system determines state parameters for each of the patients from the patient answers. The computer system processes assessments of each of the patients utilizing the answers and/or state parameters for any given patient and the medical information of the given patient. The computer system automatically generates action parameters for treating each of the patients based on the assessments of the patients. The computer system generates groupings of the state parameters of a given patient with the action parameters of the given patient for storing in the database. Included is the step of automatically categorizing patients into categories of patients having similarities to each other. The computer system updates the action parameters of the particular patient for storing in the database, the updating step including consideration of the action parameters of the patients in the same category as the particular patient. The computer system automatically generates a report including the updated action parameters for treating the particular patient.

Specific features of this aspect can include the step of the patient accessing one or both of the assessment of the patient and the report. The patient answers to the questions can comprise:
information regarding self-assessments of the medical and physiological condition of each of the patients,
information regarding the psychological condition of each of the patients,
information regarding the personality traits, communication style, genetic factors, and/or behavior patterns of each of the patients, and
information regarding fitness, activities, and/or lifestyle of each of the patients.

The information of the self-assessments of the patients can include information regarding blood pressure, cholesterol and lipids, and blood glucose levels of the patients. The assessments can be made by determining deviations between the answers and/or state parameters of a given patient and the medical information of the given patient. The report can include need-for-action levels indicating an urgency in a need for action based on the determined deviation.

Even still yet another exemplary aspect of the present technology can feature a method for individualized treatment of a patient having diabetes, including providing the patient with a questionnaire including questions pertaining to groups of success factors, wherein the questions in each success factor group pertain to at least one of biomedical information, psychological information, social environment information and personality trait information about the patient, the biomedical information including information about the patient's blood pressure, lipids and blood glucose levels. The patient provides answers to the questions of the questionnaire. A report can be provided to the patient based on the patient's answers, rating the patient's condition in each group of success factors. A doctor can convey to the patient information that is suitable for diabetes care of the patient based on the report, which brings about patient-driven behavior change affecting the patient's survival.

A specific feature that may be part of the method of this aspect is wherein the diabetes care addresses biomedical development of the patient pertaining to at least one of the following: eating, weight, physical activity, support groups, blood glucose, HbA_{1c}, lipids, variability of hyperglycemia and hypoglycemia, smoking, and general health state.

Still another exemplary aspect of the present technology can feature a method for individualized treatment and collaborative care of diabetes, including providing a patient with a questionnaire including questions pertaining to groups of success factors for the patient. The questions to each success factor group pertain to at least one or any combination of (1) biomedical information (Bio-Marker), (2) psychological information (Psycho-Marker), (3) personality trait and genetic factors information (Perso-Marker), and (4) social environment and socio-economic information (Socio-Marker) about the patient including information about the patient's blood pressure, lipids and blood glucose levels. The patient provides answers to the questions of the questionnaire, supported by a view including partner, family, friends, diabetes group(s), medical group(s), health care professionals (HCP's) and diabetes team to work out an Individualized Action Plan A series of reports are provided to the patient based on the patient's answers with the facts: (1) a report rating the patient's condition in each group of success factors, (2) a report to support furthermore detailed self-assessment, (3) a report categorizing Need for Action and interpreting the results. The Individualized Action Plan is developed with the active engagement and involvement of the patient. The HCP conveys to the patient information that is suitable for individual support and accompanying therapies for diabetes care of the patient based on the reports, which bring about patient-driven behavior change affecting the patient's survival.

Regarding the Psycho-Marker, it is known that there is an impact of depressive symptoms on adherence, function, and costs. Compared with patients in the low-severity depression symptom tertile, those in the medium- and high-severity tertiles were significantly less adherent to dietary recommendations. Patients in the high-severity tertile were significantly distinct from those in the low-severity tertile by having a higher percentage of days in nonadherence to oral hypoglycemic regimens (15% vs 7%); poorer physical and mental functioning; greater probability of having any emergency department, primary care, specialty care, medical inpatient, and mental health costs; and among users of health care within categories, higher primary (51% higher), ambulatory (75% higher), and total health care costs (86% higher). (see Ciechanowski, P. S. et al., “Depression and Diabetes: Impact of Depressive Symptoms on Adherence, Function, and Costs”, JAMA Internal Medicine, November 2000, http://archinte.jamanetwork.com/article.aspx?articleid=485556)

Major depression was associated with less physical activity, unhealthy diet, and lower adherence to oral hypoglycemic, antihypertensive, and lipid-lowering medications. In contrast, preventive care of diabetes, including home-glucose tests, foot checks, screening for microalbuminuria, and retinopathy was similar among depressed and nondepressed patients.

Regarding the Perso-Marker, it is known that the global prevalence of diabetes differs for personality traits influenced by age and culture. In developed countries the majority of people with diabetes are in the 65+ age rage. For developing countries the highest number of people are in the 45-64 age group.

Regarding the Socio-Marker, it is known that prevalence of diabetes type 2 is correlated to the socio-economic status of the patient. Lower income classes (1.10-1.15 for income quintile 1-3) have a higher diabetes prevalence than higher income classes (1.0-1.01 for income quintile 4-5).

A specific feature of this aspect can include the step wherein the diabetes care including the Individualized Action Plan, an Individualized Support Program, and an Individualized Treatment Scheme addresses biomedical development of the patient pertaining to at least one of the following: healthy and controlled eating, weight, physical activity, support groups, blood glucose (HbA_{1c}), lipids, cholesterol, control, of hyperglycemia and hypoglycemia, smoking, positive energy/burnout prevention, coping with diabetes, and general health state.

The following are directed to other exemplary aspects of the present technology.

The systems, methods, and computer programs of the present technology can be protected by an individualized personal access code (PAC) which has the quality of a cryptomized access by using the data of a Socio-psychological Fingerprint:
using all the personal data of the existing health care system for the feasibility and good practical use within the respective medical system (varying from country to country);
using the data of the Individual Personal Profile including the communication and interaction style and intimate personal data;
using the geographic situation and the global position system (GPS) as well as other electronic systems for protection of the intimate personal sphere;
thus cryptomizing the access in the highest possible degree of protection, offering strategic partnership with this example embodiment to the existing social networks, which are altogether lacking this feature of intimacy on part of their clients and users.

All of the information to the users of the system, methods, and computer programs described in the claims will be unique and person-centered by means of the InterPersonal Style (IPS)© system which in the United States of America is developed specifically for diabetes management as ‘I-D-E-A’ (Introspective-Driver-Expressive-Amiable) system:
first analyzing the IPS style (for ILM application) or ‘I-D-E-A’ style (for diabetes management and chronic disease management) of the individual (patient);
then addressing all respective informations (reports for the diabetes patient in the respective style, i.e., short, concise, and fact-oriented for the Driver, i.e., differentiated, precise, and well elaborated with many facts and figures for the Introspective with his analytic style) to be applied to all users (i.e., health care clients=patients and Health Care Professionals).

The system, methods, and computer programs of the present technology can describe the present situation and are using presently existing data and information.

In an additional example embodiment, the system, methods, and computer programs of the present technology can be applied to a large sample of individuals, using anonymized data (e.g., 20 million insurance clients with their anonymized health care reports and medical history), protected for each one of the individuals by the cryptomized PAC, the Socio-personal Fingerprint;

thus elaborating with a representative sample of a very large group of individuals (e.g., 20 million of health care clients being insured in a country) from 10 years in the past (e.g., 2002-2012) the paradigms of health development, disease development, success or failure of prevention programs, medical support programs, treatment schemes, and medical therapies;
thereby evaluating the opportunities and risks of self-care, support programs, and treatment schemes calculating the economic consequences in terms of direct and indirect costs;
in order to elaborate predictive models with the help of the third generation and patent-protected segmental, so called activity-based ILM Neural Network System of Prof. Dr. Matthias Reuter;
which are applied and integrated into the ILM system, Individualized Health Management (IHM) system, and Diabetes Management system in order to predict the chances and risks of the individuals for the future ten years (e.g., diabetes patients as to risks of macrovascular diseases, cardio-attack, stroke, co-morbidity, multi-mordity, and showing the plausibility and chances for success of individualized self-care programs, individualized support program, and individualized treatment schemes with the combined economic effects on the individual and in total on the respective health care insurance or health care company, or the national health care system.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel individualized and collaborative health care system, method and computer program that has all of the advantages of the known systems and methods, and none of the disadvantages.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a schematic diagram of an example for a collaborative care system in diabetes management of the present technology.

FIG. 2 is a schematic diagram of an example for another area of application of the Individualized Finance Management (IFM) of the present technology.

FIG. 3 is a schematic diagram of an example of the State Parameters, Action Parameters and Success Criteria of the present technology.

FIG. 4 is a schematic diagram of an example of the diabetes management system for cost-steering reimbursement management showing the 3 levels (I, II and III) of the Diabetes Reimbursement System of the present technology.

FIG. 5 is a schematic diagram of an example of the summary of benefits the diabetes management system of the present technology.

FIG. 6 is a schematic diagram of an example of the six key benefits of the diabetes management system of the present technology.

FIG. 7 is a schematic diagram of an example of advantages of the present technology for Individualized Health Management (IHM).

FIG. 8 is a schematic diagram of an example of the step collaborative care system (Example for IHM System) of the present technology.

FIG. 9 is a schematic diagram of an example of three stages of the present technology model for individual diabetes management.

FIG. 10 is a schematic diagram of an example of four steps of the present technology model for individual diabetes management.

FIG. 11 is a schematic diagram of an example of the communication system and apparatus utilizable in the present technology for input, processing, storage and output.

FIG. 12 is a schematic diagram of an example of paradigm shift in individualized and collaborative health and diabetes management of the present technology.

FIG. 13 is a schematic diagram of an example of four vectors individualized diabetes management and public cost transfer.

FIG. 14 is a schematic diagram of an example of predictive models of the present technology by activity-based Neural Network Systems.

FIG. 15 is a schematic diagram of an example of three phases of development for IHM model.

FIG. 16 is a sample screen shot of a questionnaire example of self-assessment (GUIDE) of a diabetes Type 2 Patient showing an internet welcome page and information of the present technology.

FIG. 17 is a sample screen shot of a questionnaire example of Doctor's assessment (GUIDE) of the present technology.

FIG. 18 is a sample screen shot of a questionnaire example of assessment for Patient and Doctor with comments utilizing Patent's self-assessment and Reality Check by Doctor for collaborative care.

FIG. 19 is a sample screen shot of an example of a report from self-assessment.

FIG. 20 is a sample screen shot of an example of an additional report from self-assessment.

FIG. 21 is a sample screen shot of an example of an electronic survey Personal Portfolio Page (PPP) of the present technology.

FIG. 22 is a schematic diagram of an example of four step collaborative care system of the present technology.

FIG. 23 is a sample screen shot of an example of the portfolio system of the present technology.

FIG. 24 is a sample screen shot of an example for a choice of data source.

FIG. 25 is a sample screen shot of an example for multiple chore choices, with Core 1 selected in the exemplary.

FIG. 26 is a sample screen shot of an example of the creation of the characteristic vector.

FIG. 27 is a sample screen shot of an example of a Neuronal Network System (NNS) after conditioning, showing an example success factor 1.

FIG. 28 is a sample screen shot of an example of a result of the categorization of success factor 1.

FIG. 29 is a sample screen shot of an example of a model specific adjustment of the categorization parameter, with an adjustment of the categorization radius shown for a neuron radius of 7 neurons around each winner neuron and a Cartesian distance of 1.

FIG. 30 is a sample screen shot of an example of a result of a neural categorization according to a model of the present technology.

FIG. 31 is a sample screen shot of an example of a model-specific adjustment of the categorization parameter for the evolution of a more sensitive model for specific analyses of single groups.

FIG. 32 is a sample screen shot of an example of a result of a NNS categorization according to a more sensitive model of individual analysis after group categorization showing a ‘Need for Action’.

FIG. 33 is a sample screen shot of an example of a model-specific adjustment of the categorization parameter for the evaluation.

FIG. 34 is a sample screen shot of an example of a result of a NNS categorization with additional differentiation.

FIG. 35 is a functional block diagram for a computer system implementation of the present example embodiment showing the data flow and the calculation steps/results in a flow diagram.

FIG. 36 is a functional block diagram for a computer system implementation of the present example embodiment.

FIG. 37 is a functional block diagram for an alternative computer system implementation of the present example embodiment.

FIG. 38 is a schematic block diagram of an alternative variation of the present example embodiment related processors, communication links, and systems.

FIG. 39 is an exemplary man machine interface for a patient's record and the BG prediction over 5 days.

FIG. 40 is an exemplary structure of a neural net used for modeling a patient.

FIG. 41 is an exemplary principle structure of the appropriated hierarchical net structure for predicting the BG level of a patient.

FIG. 42 is an exemplary topological form of a closed neural net structure.

FIG. 43 is an exemplary activity pattern of a closed neural net structure representing a patient's behavior in the context of diabetes.

FIG. 44 is an example for a smooth and continuous change of the activity pattern of a closed neural net structure if the patient's state changes with time t.

FIG. 45 is an exemplary modeled patient's physiological time behavior coded in an activity pattern in dependence of given insulin doses and ingested carbohydrates.

FIG. 46 is an example of predicted BG records; left-hand side: not well adapted, right-hand side: well adapted.

FIG. 47 is a schematic diagram showing how communication in the system is constituted.

FIG. 48 is a schematic diagram showing how report templates and graph templates are related.

FIGS. 49-51 together constitute a flowchart describing the interaction between patients and health care professionals within the system.

FIG. 52 is a schematic diagram showing of an Individualized Multiple Sclerosis (IMS) management plan.

FIG. 53 is a schematic diagram showing the utilization of MPPS and movement data in the creation of Bio, Psycho and Medical status.

The same reference numerals refer to the same parts throughout the various figures.

### DETAILED DESCRIPTION OF EMBODIMENTS OF THE PRESENT TECHNOLOGY

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

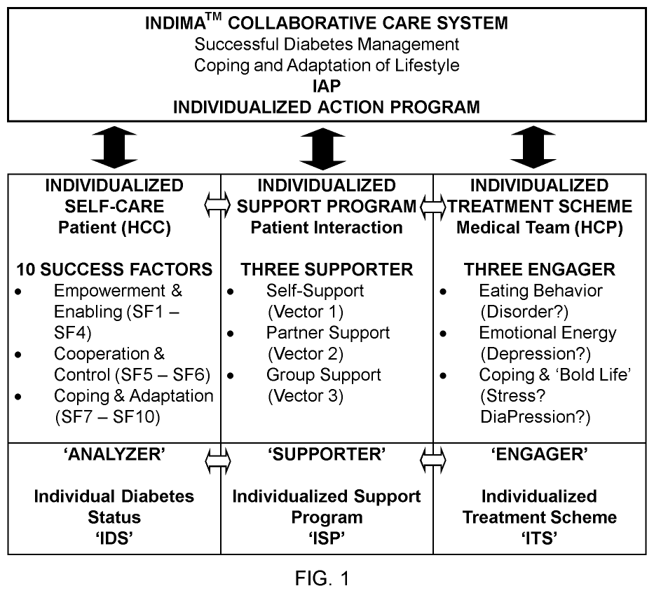

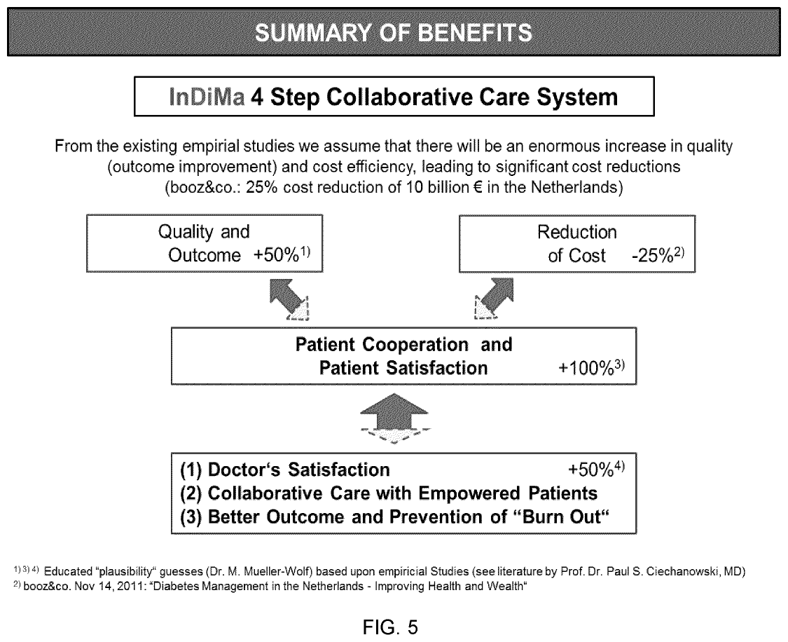

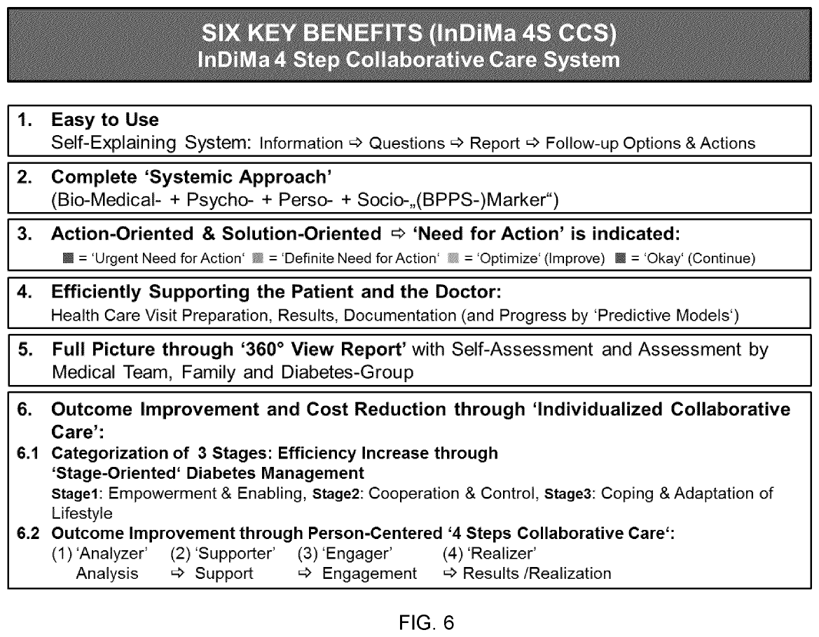

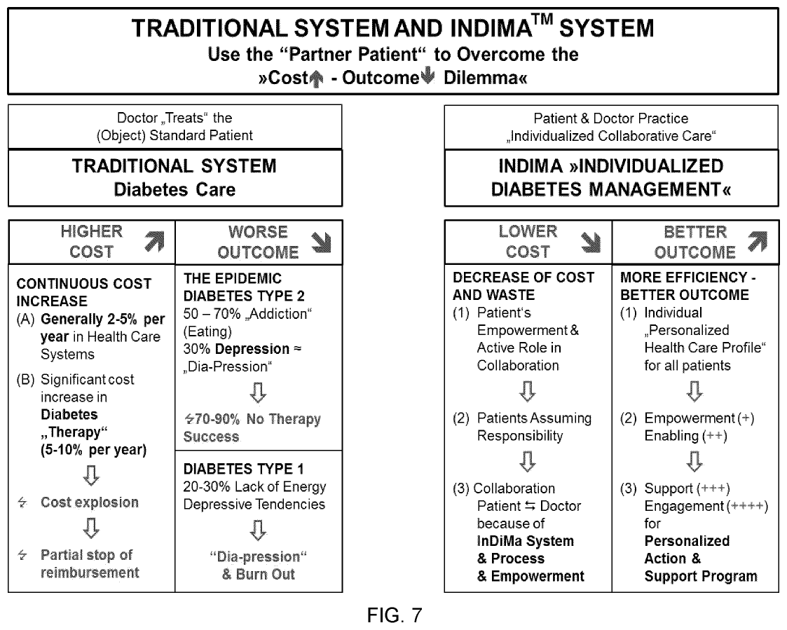

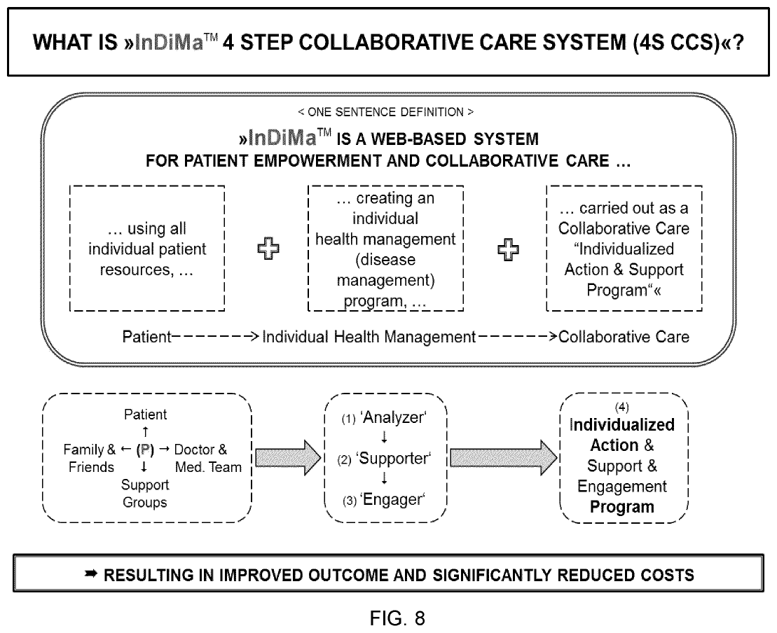

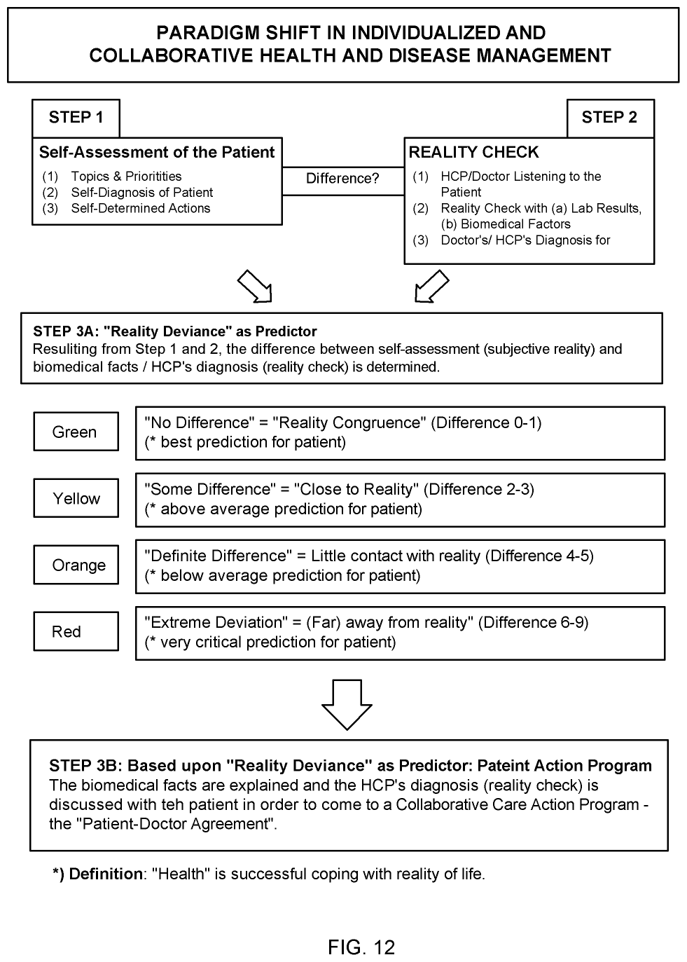

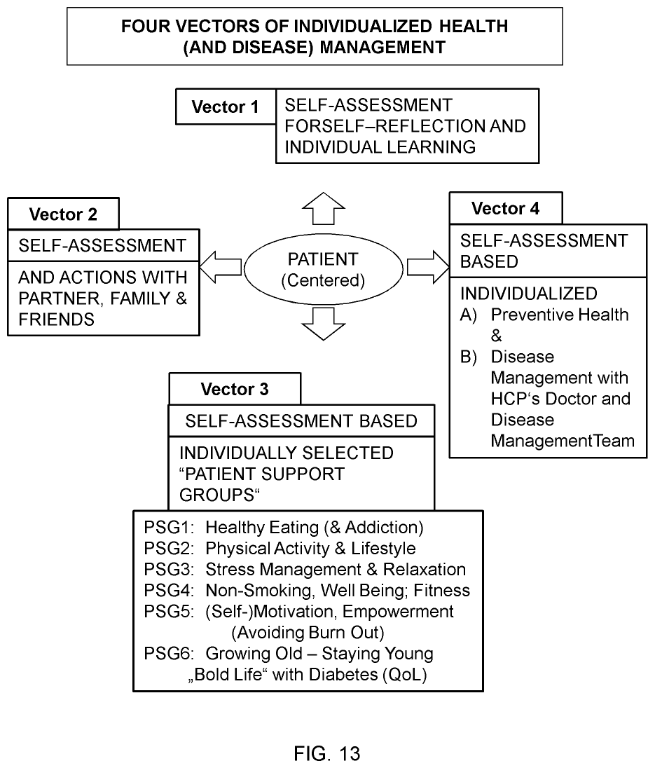

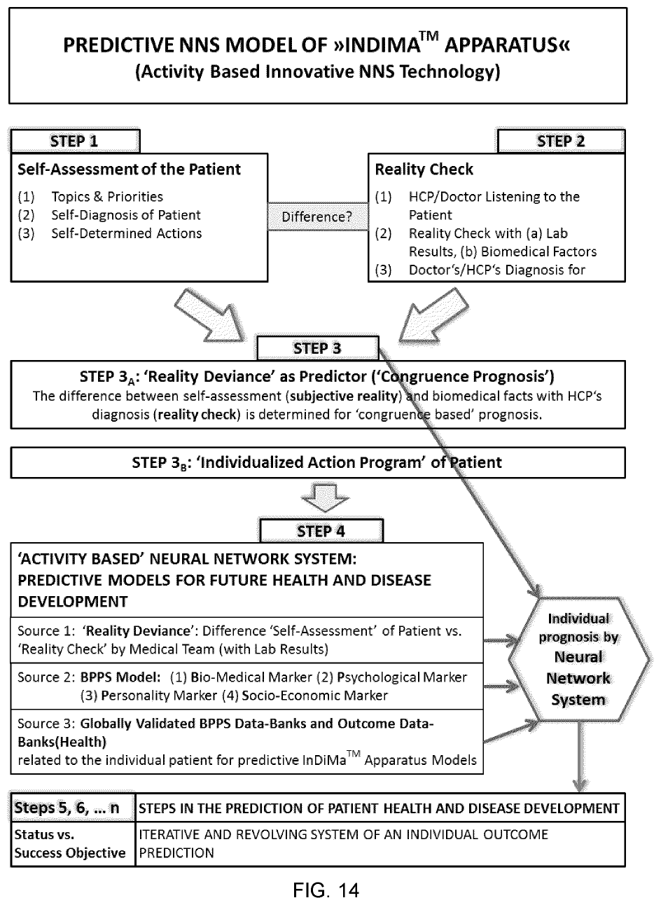

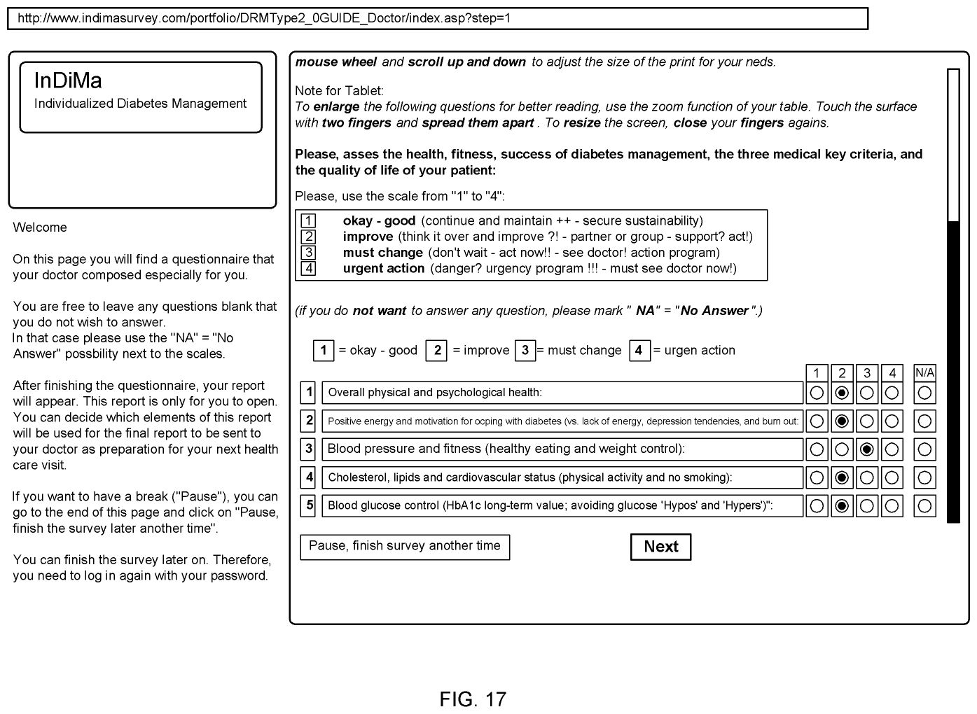

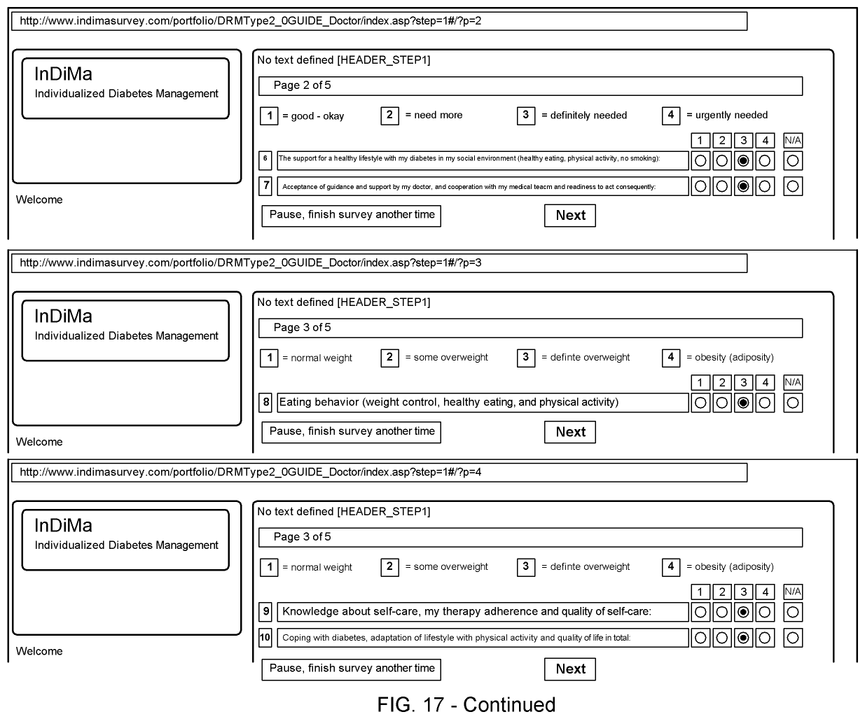

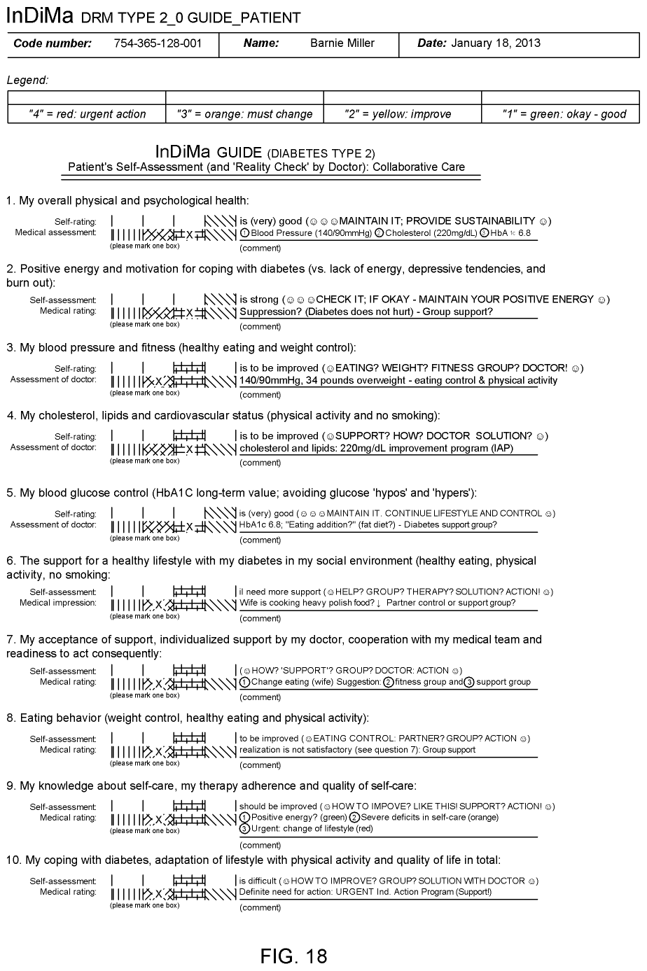

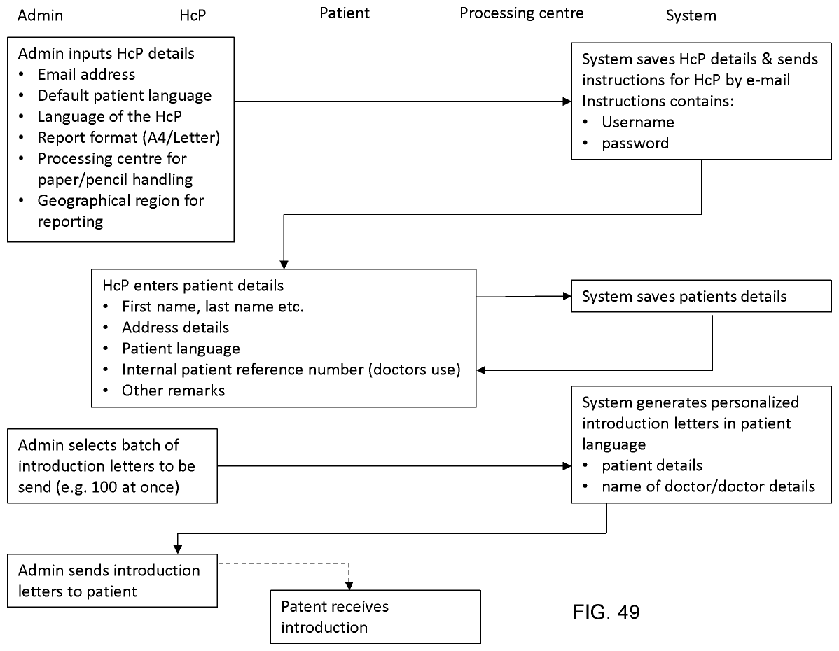

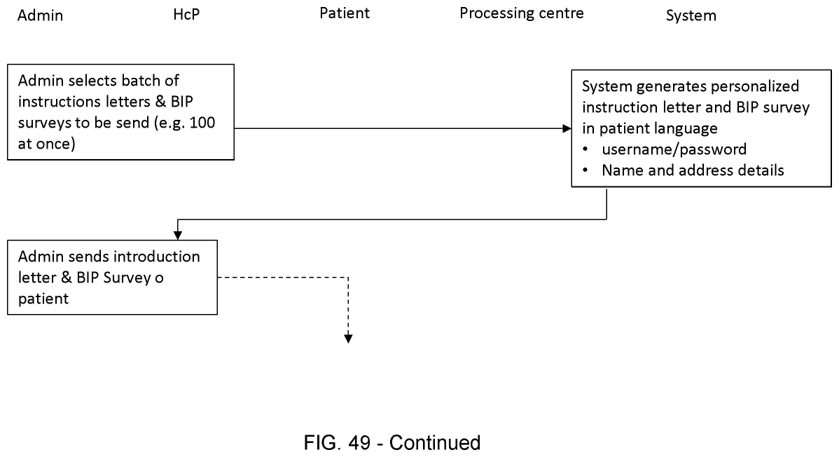

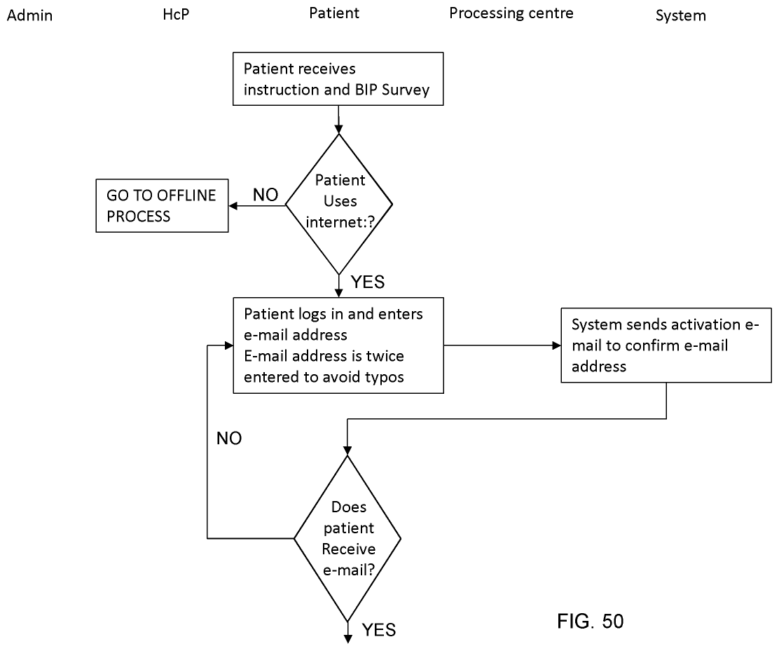

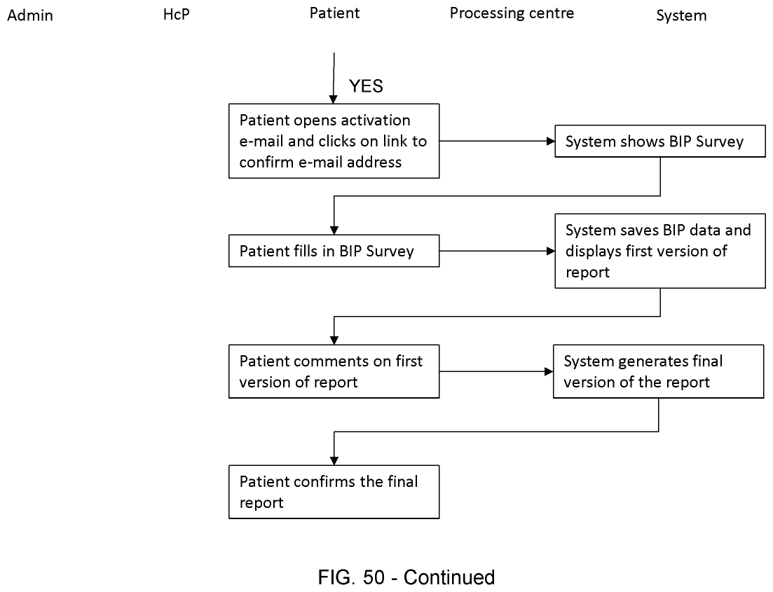

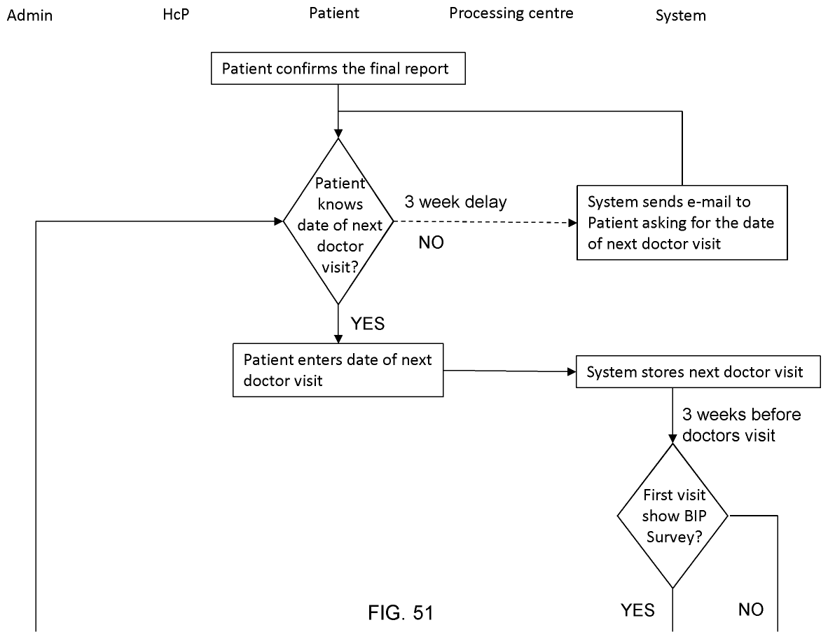

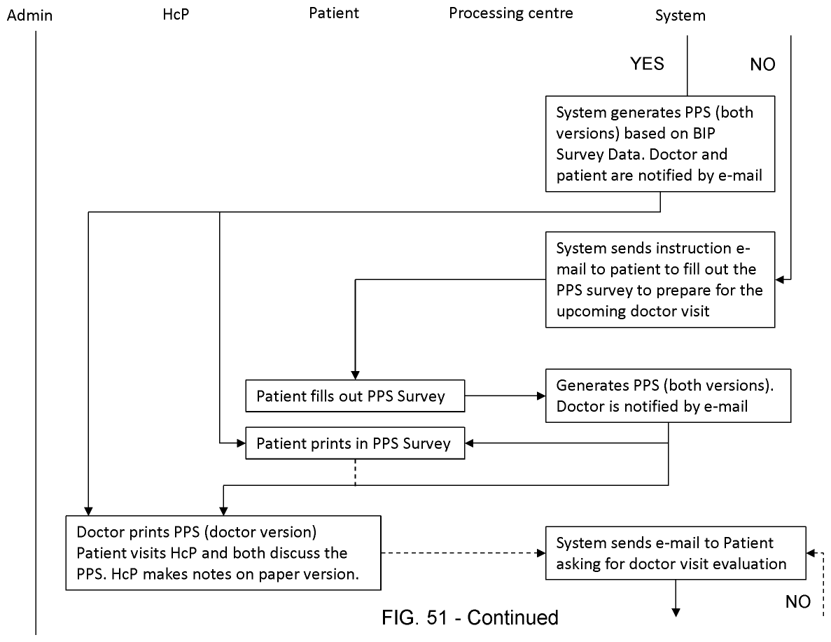

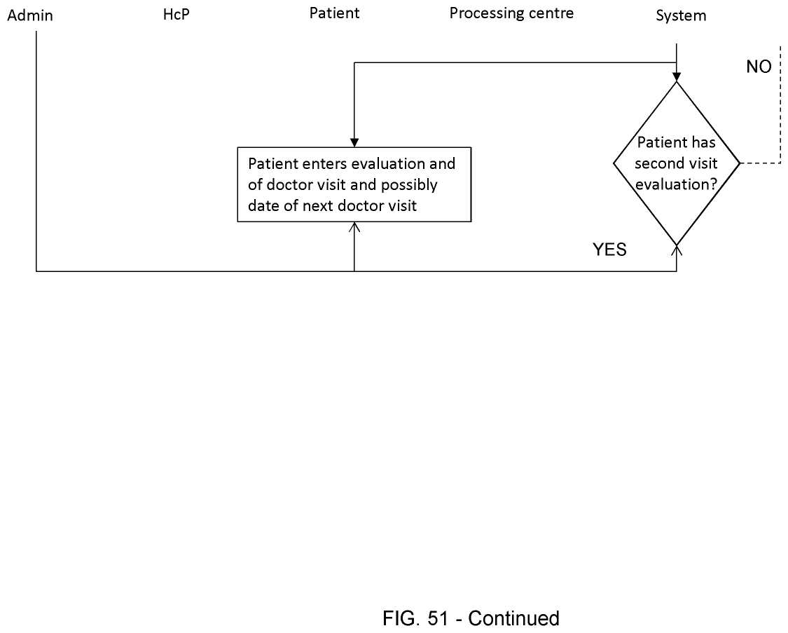

Referring now to the drawings, and particularly to FIGS. 1-53, embodiments of the present technology is shown and will be described.

FIG. 1 describes the present technology used to create the Individualized Action Programs (IAP) with the Individualized Self-Care (ISC), with the Individualized Support Program (ISP), and with the Individualized Treatment Scheme (ITS) for the collaborative care system.

An example for an area of application of the present technology can be utilized as individualized finance management (IFM). In order to reduce the complexity in the description of the example embodiment, the focus of the presentation is on the area of ‘Individualized Health Management’. The three basic modules (S=‘Socio-marker’, P_{E}=‘Perso-marker’ and P_{S}=‘Psycho-marker’) are identical for all 6 areas of the application (see Table 1 below).

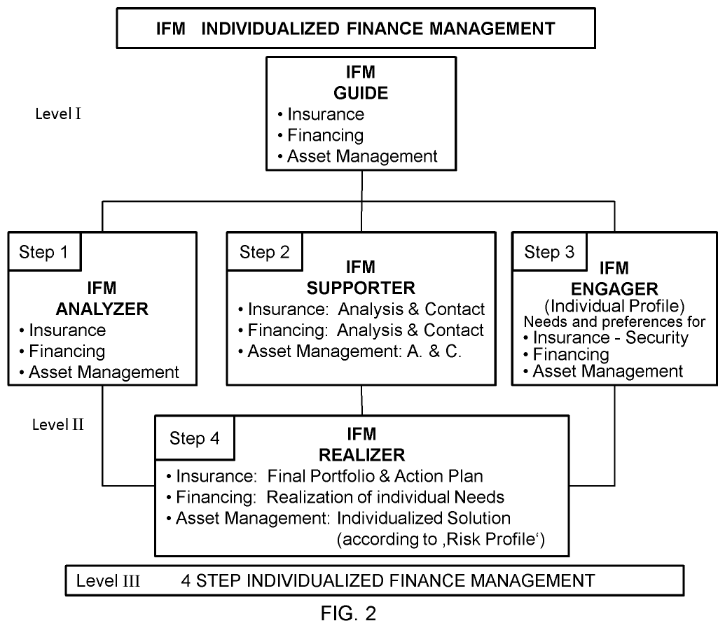

As an example for another area of application the IFM is shown in FIG. 2, which can include two levels, Level I being an IFM Guide and Level II being an IFM subguides.

Regarding Level I, the client makes a self-assessment for the three aspects describing his financial management status:
Insurance
Financing
Asset Management

Regarding Level II, which can be derived from the results of the IFM GUIDE of Level I, the client is then doing the self-assessment for the:
ANALYZER, consisting of Insurance, Financing and Asset Management
SUPPORTER, consisting of Insurance (Analysis & Contact), Financing (Analysis & Contact) and Asset Management (Analysis & Contact)
ENGAGER, consisting of Needs and preferences for Insurance (& Security).

### Financing and Asset Management

This leads to an Individualized Finance Management Action Program (=REALIZER), which is developed by the client—in the role of a partner and not of an ‘object’ of ‘manipulative treatment’ by the banker or financial consultant. The client is fully empowered, has access to an Individualized Finance Management Information System, to a Social Network of Finance Management and is associated with IFM consulting partners, focusing on Empowerment & Enabling of the client (stage 1), on Cooperation & Consensus (stage 2) and on coping with financial challenges and IFM (stage 3).

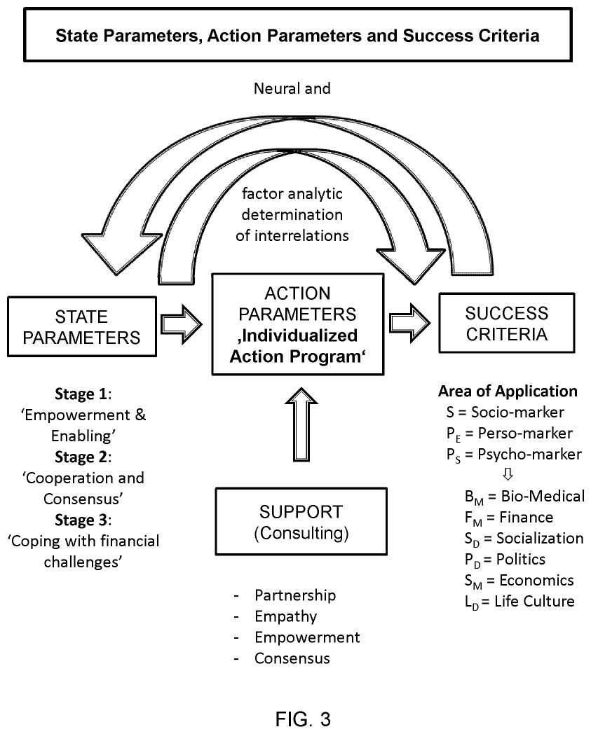

Referring to FIG. 3, a brief description of the state parameters, action parameter and success criteria will be described. All 6 areas of the ILM-Model are comprising state parameters, action parameters and are directed toward success criteria of socio-economic nature (S), of personal nature (P_{E}) and of psychological quality (P_{S}) with additional moderating parameters like support, consulting and information systems.

The state parameters are describing the status of an individual with regard to whatever is relevant for module 1 (=area of application):

Module 4 (S)=the ‘Social Marker’ describes the status of an individual in respect to the social situation, social history, social environment and socio-economic status, social and financial status and challenges of the individual.

Module 3 (P_{E})=the ‘Perso Marker’ describes the status of an individual with regard to the personality traits, the characteristics of the person, the personal life history, the communication and interaction style, the preferences, customs and habits which the individual acquired during his lifespan up to the present day.

The communication and interactive style of an individual with others can be characterized by the IPS-System (InterPersonal Style©) for the ILM areas or the ‘I-D-E-A System’, identifying the communication style and interaction style of the individual:
I=introspective
D=Driver
E=Expressive
A=Analytic

Module 2 (P_{S})=the ‘Psycho-Marker’ describing the status of an individual, the Individual Personal Profile (e.g., InstApp 001 and 021 of the 4 Step Collaborative Care System of the present technology) with the psychological make up of the individual, energy level, psychological status, stress management, personal preferences, likes and dislikes.

Module 1 is applying the ILM (Individualized Life Management) approach to the following six areas:
IHM=Individualized Health Management (Bio-Medical)
IFM=Individualized Finance Management (Finance)
ISD=Individualized Social Development (Socialization)
IPD=Individualized People Development (Politics)
ISM=Individualized Sales & Marketing (Economics)
ILD=Individualized Lifestyle & Design (Life Culture)

The action parameters are related to the modules of the state parameters:

Module 4 (S)=the ‘Social Marker’: takes into account the social situation, social history, social environment and socio-economic status, social and financial needs and challenges of the individual for the Individualized Action Program for Individualized Diabetes Care (or the respective ILM area).

Module 3 (P_{E})=the ‘Perso Marker’: individualizing the approach in the specific area and taking into account the personality traits, the characteristics of the person, the personal life history, the communication and interaction style, the preferences, customs and habits which the individual acquired during his lifespan up to the present day in order to develop an individualized, resourceful and realistic Individualized Action Program.

Module 2 (P_{S})=the ‘Psycho-Marker’: The individualization of the approach should be specific to the situation and to the person as well as to the psychological situation. Whatever the area of application is, the psychological profile in this respect (whether it is an aspect of risk management in financial management, an aspect of ethical values of Social Development of the youngsters in the family, an aspect of personal, cultural and design preferences in selecting the furniture for an apartment or making the decision of a design of an outfit)—all this has to be considered and will be integrated into the Action Program in order to individualize it according to Module 2.

Regarding Module 1 and the areas of application, whatever the existing situation (state parameters) is and whatever the success criteria or goals of the individuals are: The action parameter have to take this into account and have to create a solution which is integrating a realistic assessment of the existing social and economic situation in a specific environment at a given time (‘Reality-Check’), taking into consideration the resources of the individual (vector 1) with partner, family and friends (vector 2) and the resources that an individual can make use of in his environment (vector 3) in order to establish realistic and Individualized Action Programs with a high probability of success.

Regarding the success criteria and success factors, there should be an integration of reality (what is possible by considering the situation and the given resources).

The subjective situation and the individual resources of the person (patient/client) should be taken into account.

The resource management, using support, consulting, individual resources, resources in the near social environment and the community are also to be considered in order to develop a success-related (goal-directed) and realistic scenario.

The Individualized Actions Programs can include the steps of:

State Parameters

Action Parameters
Success Criteria or objectives and goals based upon a creative, but also realistic resource management are appropriate in order to reach the optimum for the Individualized Action Program in the respective area.

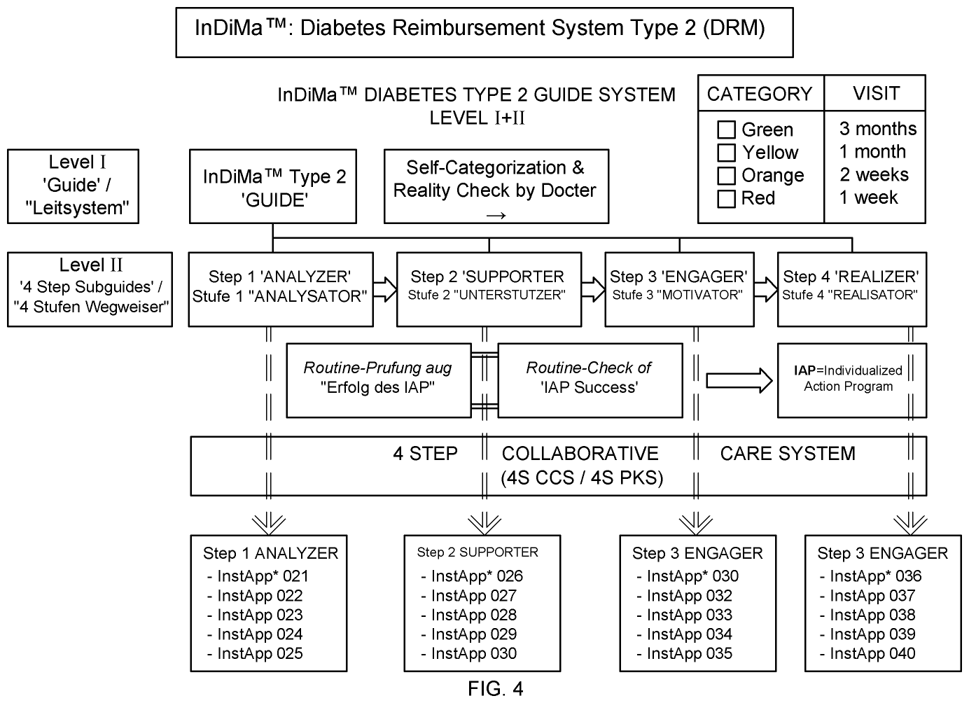

With reference to FIGS. 4-10, the example embodiment relates to a system, method, and computer program product for individualized and collaborative health care involving a plurality of individuals, using groups of state parameters that define a state of each individual, and using groups of action parameters that define (self-)treatment options and/or behavior support options of the present technology therapy and support system, targeted at an individual within said plurality of individuals.

Regarding the IHM and the present technology for today's health care systems. In recent years, collaborative health care has become an issue of increasing importance in many regions of the world, particularly in highly developed countries such as Northern America (USA, Canada) and Europe with aging populations and (nutrition and lifestyle related physical activity) effects on individuals' health.

The system, method and computer program of the present technology for Individualized Life Management and ‘Individualized Health Management, can be described with the example embodiment that applies to all relevant aspects of life management and to the six areas of Individualized Life Management (see Table 1).

TABLE 1

| 4 Modules | ILM = Individualized Life Management | Area |
|---|---|---|
| (1) S [symbol] P_{E} [symbol] P_{S} [symbol] B_{M}: | IHM = Individualized Health Management | (Bio-Medical) |
| (2) S [symbol] P_{E} [symbol] P_{S} [symbol] F_{M}: | IFM = individualized Finance Management | (Finance) |
| (3) S [symbol] P_{E} [symbol] P_{S} [symbol] S_{D}: | ISD = Individualized Social Development | (Socialization) |
| (4) S [symbol] P_{E} [symbol] P_{S} [symbol] P_{D}: | IPD = Individualized People Development | (Politics) |
| (5) S [symbol] P_{E} [symbol] P_{S} [symbol] S_{M}: | ISM = Individualized Sales & Marketing | (Economics) |
| (6) S [symbol] P_{E} [symbol] P_{S} [symbol] L_{D}: | ILD = Individualized Lifestyle & Design | (Life Culture) |

In the following, we are focusing on the Individualized Health Management and especially on the present technology model for Individualized Diabetes Management, which applies also for patients with other chronic diseases, acute disease management, and Individualized Wellness & Prevention of the IHM Model for Individualized Health Management.

To show the practical relevance and the benefits of the example embodiment with system, method and computer program of the example embodiment, we are focusing on chronic diseases and especially on Individualized Diabetes Management utilizable with the Individualized Health Management System of the present technology.

In an exemplary, the present technology can be described for diabetes and lifestyle. It is well-known that some widely spread and frequently occurring chronic diseases (especially diabetes) are closely-related to Western lifestyle with unhealthy nutrition patterns. Over nutrition, combined with sedentary lifestyle, typically leads to overweight and obesity which is likely to cause acute macrovascular problems and threats (macrovascular diseases like diabetes, cardio-attack, and stroke) to a an individual's health or even life.

It can be appreciated that there is no adequate individualized and collaborative health care for diabetes and other chronic diseases. Depression, combined with diabetes, also called “diapression”, stress and burn-out, often combined with psychosomatic diseases, gastro-intestinal diseases, chronic orthopedic diseases (head, shoulders, back), chronic pain-related diseases and all other chronic diseases need to be understood and treated in a holistic approach. This means to incorporate the body (bio-medical aspects), the soul (psychological aspects), the personality (character and genetic disposition), and the social history, socio-economic status and social environment of the patient(s).

Increasing diabetes-related and health care-related economic burden. As a result, these individuals' quality of life—in the case of diabetes type 2—is severely impaired both physically and mentally. In consequence, public health systems of these highly developed countries are exposed to an increasing economic, health-related, and productivity burden.

An example for these lifestyle-related problems: 66% of the US population is overweight (December 2010, US Today) and 34% are diagnosed as obese (Body Mass Index=BMI over 30).

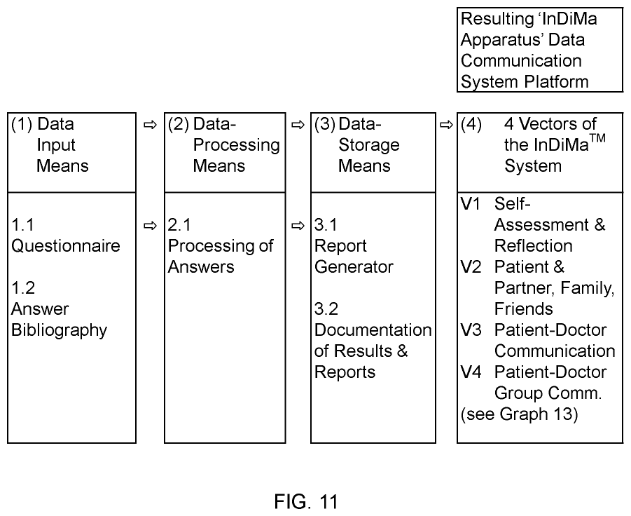

Rationally based bio-medical health care is not sufficient. With reference to FIGS. 11-14, at present, the health care systems in many highly developed countries provide a wide range of measures to address these individuals' health problems. Unfortunately, most medical treatments are based merely or primarily on an individual's easily measurable biological and medical (physiological) condition without taking into account that individual's psychological (mental and emotional) condition, the patient's personality (character and genetic factors) and the social factors (socio-economic environment).

The present technology can utilize a B-P-P-S Model for individualized diabetes management. Table 2 shows the Four Factor B-P-P-S Model which also applies to 5 other areas of the ILM System. The B-P-P-S Model for IHM and for the chronic disease management comprises the following four basic factors:

TABLE 2

| | |
|---|---|
| “Bio- | (1) the treated individual's bio-medical and physiological |
| Medical” | condition |
| [symbol] | |
| “Psycho” | (2) the individual's psychological condition |
| [symbol] | |
| “Perso” | (3) the individual's personality structure, genetic factors, and |
| [symbol] | behavior patterns and |
| “Socio” | (4) the social history and social (lifestyle) environment of the |
| [symbol] | individual |

The individual's bio-medical and physiological condition (B) is the combined result of the individual's psychological, personal, and genetic as well as social and socio-economic factors: the moderating modules P-P-S (see Table 2).
Module Psycho (P_{S}): The individual's psychological condition (P_{S}) is the dynamic aspect of a personality.
Module Perso (P_{E}): The psychological status of a person is intertwined with the individual's personality traits (character) and genetic factors (P_{E}).
Module Socio (S): The socio-economic, genetic, and social environment factors within the patient's life (S) are the basis for the personal development (P_{E}) and influence the psychological status (P_{S}).

The interrelations of the four factors B-P-P-S of the IHM and present technology model. It has been empirically proven that an individual's psychological condition is part of the soul (psyche)-body (soma) interrelation in the course of individualized and collaborative health care. The personality structure, the (during the former life acquired) behavior patterns, and the psychological state of an individual have both direct influence (e.g., psychosomatic medicine, placebo effect) and indirect influence (via modified patient behavior, i.e., treatment adherence and compliance) on the individual's physiological and medical (Bio-Med) condition.

As a result, the existing purely bio-medically oriented health care systems are significantly less successful than they potentially could be.

There is a need for the present technology for diabetes management in the exemplary. A striking example is the diabetes therapy: There is a significant need for improvement in the quality and efficiency of health care systems in general and diabetes care (especially for type 2) in specific.

Therefore, it is an objective of the present technology to improve the quality and efficiency of such health care systems, reducing the costs significantly through the active integration of the patient.

The present example embodiment of the IHM and the present technology can be a system for individualized and collaborative health care. The present example embodiment provides a system for individualized and collaborative health care involving a plurality of individuals, using groups of state parameters for defining a state of each individual, and using groups of action parameters for defining (self-)treatment options and/or behavior support options of the present technology therapy and support system, targeted at an individual within said plurality of individuals.

An exemplary function of the present technology can be for example for an IHM Apparatus. The present technology system can comprise a data processor means adapted for processing input data, which are based on said groups of state parameters, into output data, which are the basis for said groups of action (adaptation, change, and treatment) parameters, using defined relationships/options for action between groups of state parameters and groups of action (adaptation, treatment, and support) parameters; a data storage means adapted for storing said groups of state parameters, said groups of action (adaptation, treatment, and support) parameters and said defined relationships/options for action between groups of state parameters and groups of action parameters; characterized in that the system further comprises a data communication system/platform adapted for communicating state parameters selected from said groups of state parameters and/or action parameters selected from said groups of action parameters among said plurality of individuals.

Communication among categorized patients utilizing the present technology and/or the IHM Apparatus can be accomplished. Due to the data communication system, the health care system of the present technology system enables individuals, i.e. patients whose health is (self-)monitored or who undergo medical treatment and support with lifestyle adaptation, to exchange personal health-related and/or personal treatment-related information. Thus, the present technology system enables not only patients and health care professionals (medical doctors, nurse educators, and nurses) to communicate in a higher quality and more efficiency via the present technology system, but also patients to communicate with each other.

This possibility for communication and information exchange provides patients with the benefits of a (self-)support group with the extra benefit of having relevant information readily available to be exchanged among the patients.

The IHM model of the present technology can feature a Phase I including individualized empowerment and enabling of patients. Using this relevant information in the form of groups of state parameters defining a state of each patient including this patient's biological/medical (physiological) condition as well as this patient's psychological (mental and emotional) condition, personality and genetic factors as well as social and socio-economic factors and in the form of groups of action (treatment and support) parameters defining treatment and behavior support options for each patient within the plurality of patients puts each patient in an empowered, more self-determined, and strengthened psychological (mental and emotional) position. Patients aware of other patients' health conditions and life situations tend to develop significant improvements in treatment adherence and lifestyle change with significantly improved behavior patterns and compliance with behavior changes as offered by the treatment options and behavior support options offered to the patients.

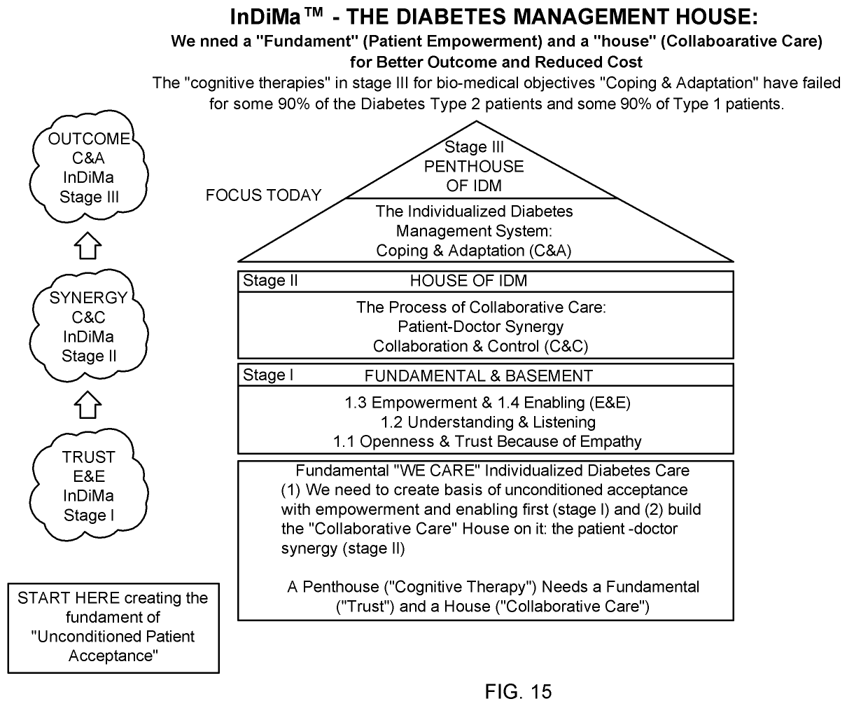

There can be three phases of development IHM model of the present technology (see FIG. 15), utilized in an example of application of the present technology. In the present technology health care system of the present example embodiment, the data processor means may comprise an adaptive structure where said defined relationships and options for action (treatment and support) between groups are redefined/updated using empirical pairs of action (treatment and support) parameter groups and state parameter (patient psychometry and patient-doctor communication) groups.

The present technology can include self-organizing maps (SOM's) of a learning system. Due to this adaptive structure or dynamic knowledge base with, for example but not limited to, SOM's, the present technology health care system of the present example embodiment is a learning system, which is constantly updated using the empirical pairs of action parameter groups and state parameter groups of patients who most recently joined the present technology health care system.

Rather, the adaptive structure (lifestyle adaptation for example, but also treatment and support options) can be selected from one or any combination of patient self-assessment as basis, from expert systems, fuzzy logic, neuronal networks, genetic and/or evolutionary algorithms and combinations thereof.

The present technology can include adaptive structures and predictive models. Adaptive structures of this kind are updated automatically by including empirical pairs of state parameter groups (patient self-assessment) and corresponding action (treatment and support) parameter groups who most recently joined the present technology health care system of the present example embodiment. Knowledge-based software structures with explicit rules such as expert systems, fuzzy logic, and combinations thereof are usable, when the number of individuals having joined the present technology health care system of the present example embodiment is rather limited. Non-knowledge-based software structures such as neuronal networks, genetic and/or evolutionary algorithms and combinations thereof are usable, when the number of individuals having joined the health care system of the present example embodiment is very large: typically more than 1000 individuals and more than 10,000 individuals; finally using data banks with millions of patients for Neuronal Network System (NNS) analyses. When stating these numbers, it should be noted that an individual application of the present technology health care system of the present example embodiment normally deals with only one type of disease and a population of individuals suffering in different individual patterns and degrees from this disease.

The present technology can be a web-based system. As a web-based system, the health care system of the present example embodiment may be accessed via a computer network such as the internet or an intranet, i.e. a computer network linking individual hospitals, health care professionals' practices or clinics, offices of support groups and patients' homes and even health care professionals' and patients' mobile communication devices.

The groups of state parameters of the present technology may be selected from one or any combination of biomedical (physiological), psychological, personal, and social (socio-economic) (“BPPS”) characteristics/attributes of health care clients. Preferably, all of these characteristics/attributes are selected for constructing the groups of state parameters.

The present technology can include an innovative neural network system (INNS) utilizable with a holistic approach. This allows each client/patient having joined the health care system of the present example embodiment to be viewed in its entirety (holistic approach). Rather than looking at an individual patient in an isolated manner, i.e. only from the medical/physiological angle, the health care system of the present example embodiment looks at the individual patient from all angles, i.e. considering various aspects that are prone to influence that individual patient's reaction when faced with treatment options and/or change-of-behavior options.

The present technology can include client-specific state parameters. Preferably, a health care client-specific state parameter group is based on assessment of a health care client using a questionnaire.

An example of 10 Guide questions is shown in Table 3.

TABLE 3
“1” = okay - good, “2” = improve, “3” = must change, “4” = urgent action

| | | | | | | |
|---|---|---|---|---|---|---|
| 1) | My overall physical and psychological health: | \textcircled{1} | \textcircled{2} | \textcircled{3} | \textcircled{4} | “NA” |
| 2) | Positive energy and motivation for coping with diabetes | \textcircled{1} | \textcircled{2} | \textcircled{3} | \textcircled{4} | “NA” |
| | (vs. lack of energy, depressive tendencies and burn out): | | | | | |
| 3) | My blood pressure and fitness (healthy eating and weight | \textcircled{1} | \textcircled{2} | \textcircled{3} | \textcircled{4} | “NA” |
| | control): | | | | | |
| 4) | My cholesterol, lipids and cardiovascular status (physical | \textcircled{1} | \textcircled{2} | \textcircled{3} | \textcircled{4} | “NA” |
| | activity and no smoking): | | | | | |
| 5) | My blood glucose control (HbA1c long-term value; | \textcircled{1} | \textcircled{2} | \textcircled{3} | \textcircled{4} | “NA” |
| | avoiding glucose hypos' and ‘hypers’): | | | | | |

“1” = okay - good (sufficient), “2” = need more (not enough),
“3” = definitely needed (too little), “4” = urgently needed (no support)

| | | | | | | |
|---|---|---|---|---|---|---|
| 6) | The support for a healthy lifestyle with my diabetes in my | \textcircled{1} | \textcircled{2} | \textcircled{3} | \textcircled{4} | “NA” |
| | social environment (healthy eating, physical activity, no | | | | | |
| | smoking): | | | | | |
| 7) | My acceptance of guidance and support by my doctor, | \textcircled{1} | \textcircled{2} | \textcircled{3} | \textcircled{4} | “NA” |
| | cooperation with the medical team and readiness to act | | | | | |
| | consequently: | | | | | |

“1” = under control (normal weight), “2” = mostly controlled (some overweight), “3” = often
uncontrolled (definite overweight), “4” = no control (obesity - adiposity)

| | | | | | | |
|---|---|---|---|---|---|---|
| 8) | Eating behavior (weight control, healthy eating and physical | \textcircled{1} | \textcircled{2} | \textcircled{3} | \textcircled{4} | “NA” |
| | activity): | | | | | |

“1” = okay - good, “2” = improve, “3” = must change, “4” = urgent action

| | | | | | | |
|---|---|---|---|---|---|---|
| 9) | My knowledge about self-care, my therapy adherence and | \textcircled{1} | \textcircled{2} | \textcircled{3} | \textcircled{4} | “NA” |
| | quality of self-care | | | | | |
| 10) | My coping with diabetes, adaptation of lifestyle with physical | \textcircled{1} | \textcircled{2} | \textcircled{3} | \textcircled{4} | “NA” |
| | activity and quality of life in total: | | | | | |

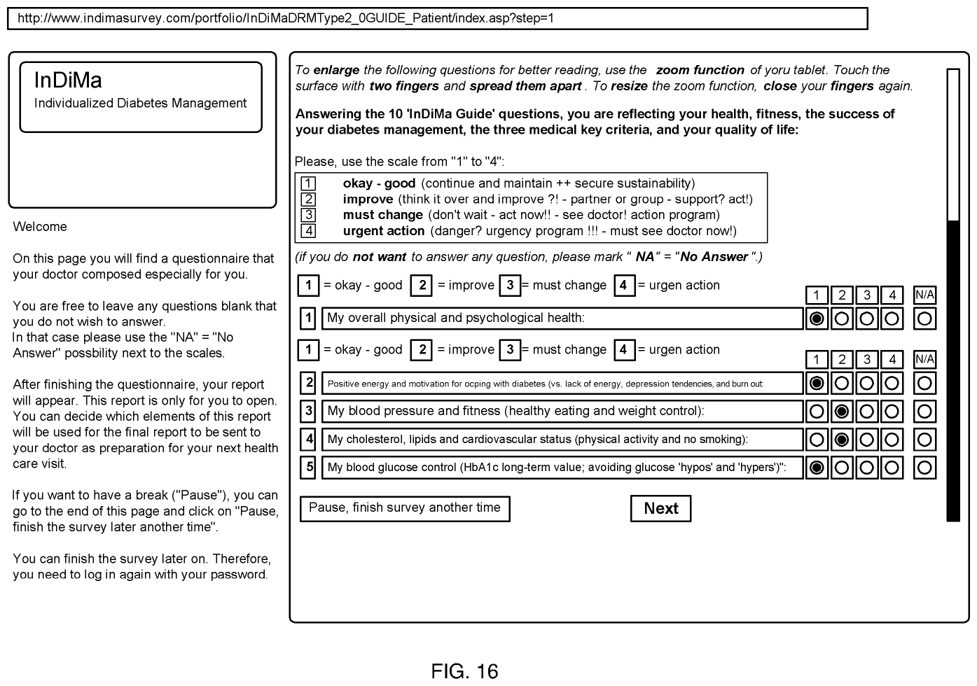

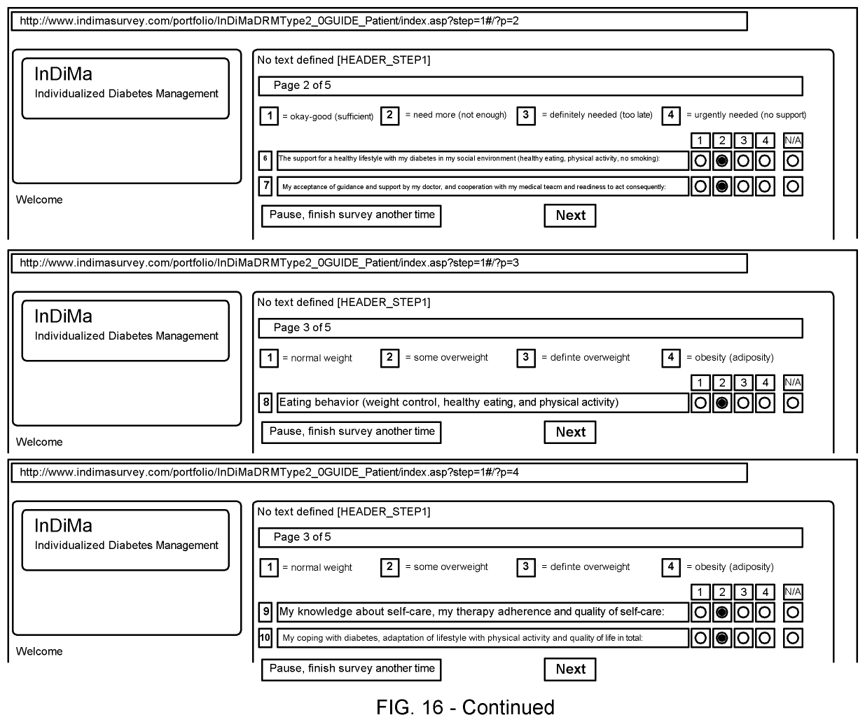

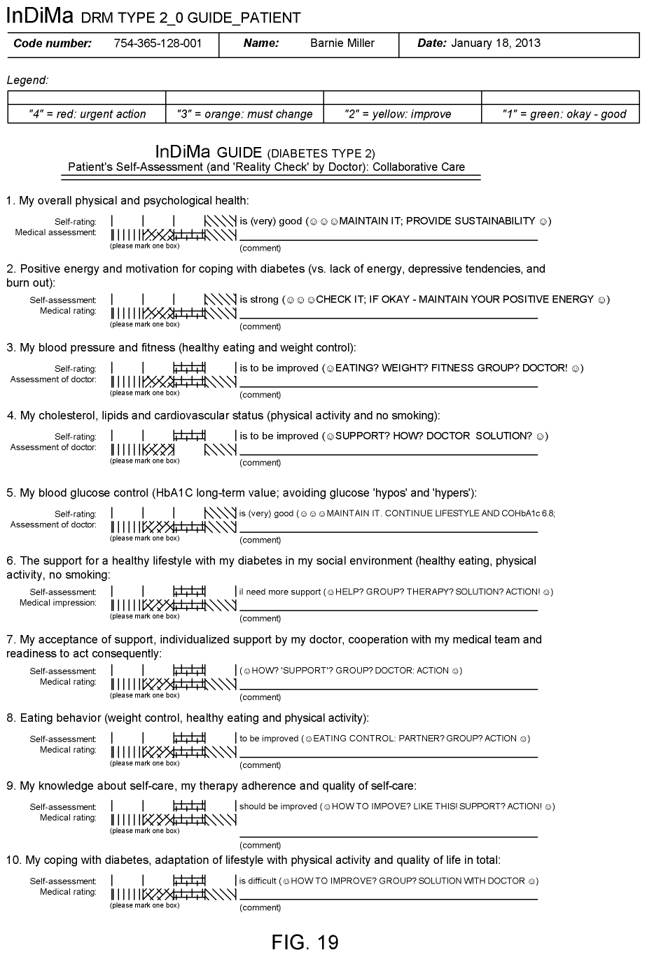

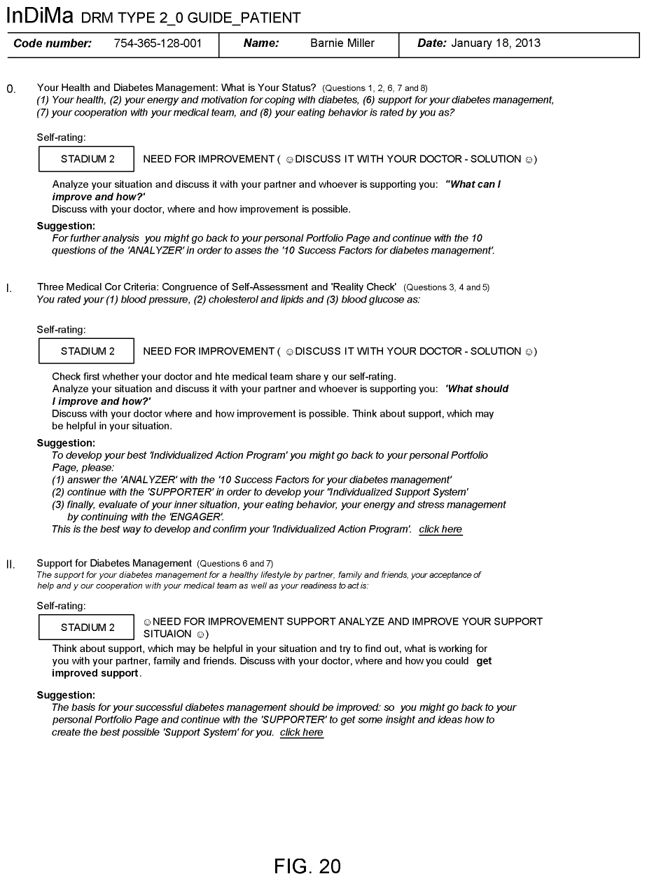

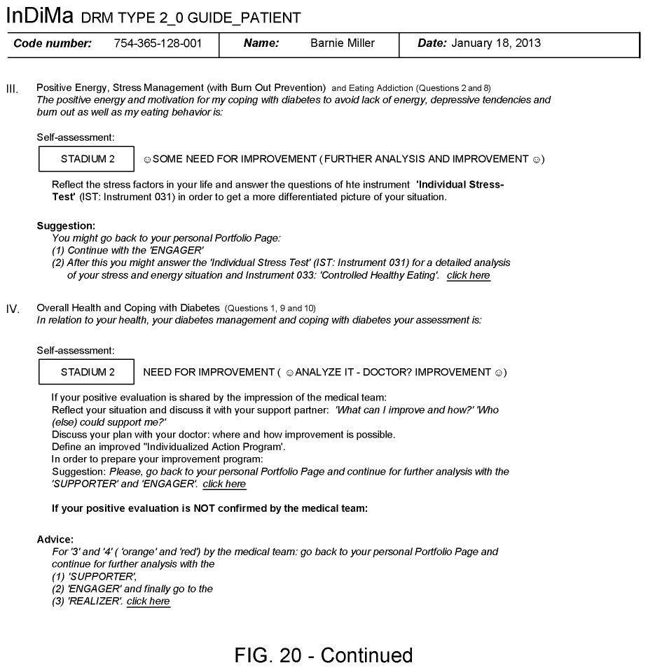

Referring to FIG. 16, an exemplary self-assessment is shown for a patient (Barnie Miller). Such assessment by questionnaire provides a standardized collection of information about the health care client. This standardized collection of information can easily be transferred into groups of state parameter values for defining a state of each individual, see FIGS. 19-20.

Preferably, the questionnaire is a web-based questionnaire which may be sent to a health care client via a computer network such as the internet or an intranet, i.e. a computer network linking individual hospitals, health care professionals' practices or clinics, offices of support groups and patients' homes and even health care professionals' and patients' mobile (wireless) communication devices (templates, including for instance iPhone technology).

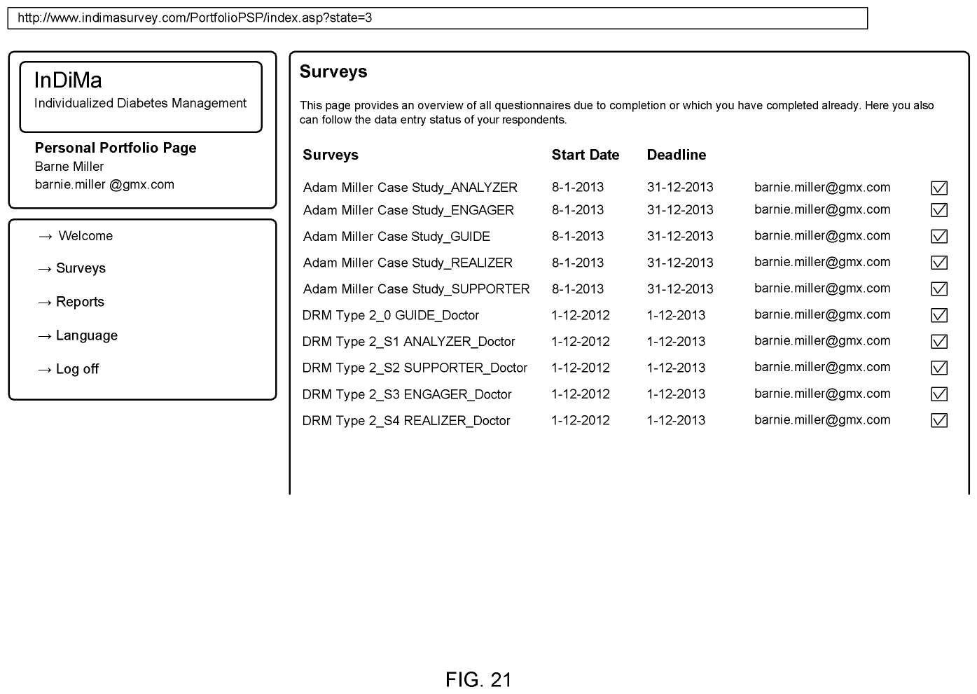

FIG. 21 is a screenshot of an exemplary electronic survey (Personal Portfolio Page).

The following is a further description of groups of state parameters of the present technology. In view of the foregoing, the present example embodiment also provides a method for individualized and cooperative health care involving a plurality of individuals, using groups of state parameters that define a state of each individual, and using groups of action parameters that define treatment options and/or behavior options targeted at an individual within said plurality of individuals, the method comprising: processing input data, which are based on said groups of state parameters, into output data, which are the basis for said groups of action parameters, using defined relationships/assignments between groups of state parameters and groups of action parameters; and storing said groups of state parameters, said groups of action parameters and said defined relationships/assignments between groups of state parameters and groups of action parameters; characterized by communicating state parameters selected from said groups of state parameters and/or action parameters selected from said groups of action parameters among said plurality of individuals.

The present technology can include information and concepts for support groups. Due to the data communication step, the health care method of the present example embodiment enables individuals to exchange personal health-related and/or personal treatment-related information. The method may include communication of patients with each other. This possibility for communication and information exchange provides patients with the benefits of a (self-)support group with the extra benefit of having relevant information readily available to be exchanged among the patients. Thus, each patient is strengthened psychologically (mentally and emotionally) and is prone to significantly improve his discipline and compliance with respect to the treatment and/or behavior changes defined by the treatment options and/or behavior options specifically targeted at him.

The following is a description of the action parameter and state parameter groups of patients. The defined relationships/assignments between groups may be redefined/updated using empirical pairs of action parameter groups and state parameter groups of patients who most recently joined the health care system of the present example embodiment. Each of the new patients whose patient information, i.e. state parameter values or patient profile, is added to the system and whose associated treatment information, i.e. action parameter values or treatment profile, is added to the system will broaden the empirical base of the system. These empirical pairs of state parameter values and their associated action parameter values are preferably evaluated by quantifying the success of the treatment and/or behavior changes for the patient they belong to. Based on this quantified success, an evaluated and weighted empirical pair, composed of state parameters with their values and action parameters with their values, is taken into consideration for modifying/updating the previously existing relationships/assignments between state and action parameter groups.

Preferably, the above groups of state parameters are selected from the group consisting of biomedical (physiological), psychological, personal, and socio-economic (“BPPS”) characteristics/attributes of health care clients.

Preferably, a health care client-specific state parameter group is determined by assessing this health care client using a questionnaire. This questionnaire may be a traditional one in paper format.

The present technology can include a driven collaborative care of health care client (HCC) and HCP. In a specific exemplary embodiment, the method can comprise assessing a health care client's specific state parameter group, i.e. the value for each state parameter in this group, using two assessment processes that are at least partially independent of each other. Typically, a first assessment process involves the HCC only, i.e. the HCC answers the questions of the questionnaire all by himself, and a second assessment process involves the HCP only, i.e. the HCP answers the questions of the questionnaire all by himself. As a first alternative, the second assessment process may involve the HCC and the HCP, i.e. they answer the questions of the questionnaire again together. As a second alternative, a third assessment process may involve the HCC and the HCP. There may be even more alternatives for additional/complementary assessment processes involving persons other than the HCC or the HCP such as the HCC's family members or friends.

Once these different assessment processes of a specific HCC have been carried out, the results of these assessment processes are compared.

The present technology can include a categorization of HCC need for action. The results of these comparisons again provide significant information for the categorization of each HCC, in particular with respect to the psychological, personal and socio-economic characteristics/attributes of this HCC and the consequent need for action.

More preferably, when the health care system of the example embodiment is a web-based system, the questionnaire is provided in email format to an authorized health care client's email address. The authorized health care client joins the health care system of the present example embodiment by answering multiple-choice questions, selecting statements, using rating scales and qualitative assessments etc. in the questionnaire. Based on this information provided by the client, each parameter in the group of state parameters is assigned a marker/value for this specific client. Based on this client-specific state parameter group and on the defined relationships/assignments between groups of state parameters and groups of action parameters, a client-specific action parameter group is generated where each parameter in the group is assigned a marker/value for this specific client: e.g., categorizing the need for action options for the Individualized Action Program for the individual patient.

Feedback and giving options can be provided by the present technology system. The authorized HCC whose individual state is now defined and included in the health care system of the present example embodiment may be given feedback by the system disclosing to him the treatment options and/or behavior options based on the system-determined action parameter markers/values. In addition, this authorized health care client may be given feedback by the system disclosing information about health care professionals for him to choose for his future treatment.

Preferably, the health care client-specific state parameter group is repeatedly determined throughout this health care client's affiliation to the plurality of individuals of the health care system of the present example embodiment.

The present technology can provide communication and collaborative care between HCC and HCP. Thus, this HCC will benefit from the accumulated information and updated defined relationships/assignments between groups of state parameters and groups of action parameters in the system gathered from additional authorized health care clients that joined the health care system of the present example embodiment after him. In other words, once each individual health care client has joined the health care system of the present example embodiment, he will benefit from his own and all other health care clients' contribution to the knowledge/intelligence of the system.

In a specific example embodiment, communication and information exchange is made available among individuals belonging to a first subset of the plurality of individuals including HCC; among individuals belonging to a second subset of the plurality of individuals including HCP; and between individuals belonging to the first subset (HCC) and individuals belonging to the second subset (HCP).

Still further, the present technology can provide patient to patient communication. Thus, the system and method of the present example embodiment enable individual health care clients/patients to enter into direct contact and communication with each other. Such communication within a group sharing some common problem(s) has been shown to help alleviate an individual's fears and frustration about his or her health situation by making that situation more acceptable (“I am not the only one having this problem”) and increasing the individual's motivation to actively contribute to an improvement of his or her situation. It has been shown that individuals in such a setting are more disciplined which makes them significantly more compliant throughout their medical treatment and encouraged behavior modifications. In other words, the system and method of the present example embodiment integrates support group functionalities with all the known benefits of a support group.

Similarly, the system and method of the present example embodiment enable individual health care professionals/medical doctors to enter into direct contact and communication with each other. Such communication among experts may be necessary for very difficult cases. Thus, the health care professionals affiliated to the system and method of the example embodiment are supported by the case-specific or client-specific action parameter values defining options for treating a that specific client and/or modifying that client's behavior.

The present technology can further provide cross-communication between HCC(s) and HCP(s). Also, the system and method of the present example embodiment enable cross-communication between health care clients on the one hand and health care professionals on the other hand. This provides additional transparency and some competition both for health care clients and for health care professionals. Increased transparency and competition among health care clients means that they tend to compete for treatment success, which translates into improved compliance during the treatment received. Increased transparency and competition among health care professionals means that they tend to compete for treatment success, which translates into adoption of best (client-specific) practices for the treatment provided. In other words, cooperation between health care clients and health care professionals towards a common goal (treatment success) is improved.

Sharing of information and empirical results of treatment options can be provided. Preferably, the defined relationships/assignments between action parameter groups and state parameter groups are made available for communication and information exchange among the plurality of individuals including health care clients/patients and among the plurality of individuals including health care professionals/medical doctors.

This allows every health care client to identify where he and is health situation and the associated treatment options are positioned within the overall spectrum of health care clients, health situations and treatment options.

Similarly, this allows every health care professional to identify his clients' health situations, the associated treatment options and to compare them with the overall spectrum of health care clients, health situations and treatment options.

Preferably, individuals of the above plurality of individuals are categorized into different categories of individuals based on their respective state parameter groups and corresponding action parameter groups (e.g., the need for action priorities).

As mentioned above, the number of individuals having joined a health care system of the present example embodiment is very large, typically more than 1,000 individuals, more than 10,000 individuals, and finally millions of patients in a global setting can be reached. It should be noted that an individual health care system of the present example embodiment typically covers only one type of disease and individuals suffering in individual patterns and to different degrees from the respective disease. Given these numbers of individuals and a finite spectrum of overall individual health situations defined by specific state parameter combinations, a representative number of health care client (HCC) categories can be defined with each category including a reasonable number of individual health care clients. This guarantees that all HCC individuals in each HCC category are similar enough to undergo one category-specific treatment on the one hand and that each HCC category includes a large enough number of HCC individuals to be attractive enough for intra-category communication and information exchange among HCC.

Also predictive models for individualized treatment options and related outcome probabilities linked to cost-benefit predictions can be developed by the present technology.

The IHM system of the present technology can include collaborative care. In summary, the following characteristics are describing the IHM System in general for the exemplary individualized diabetes management with the collaborative care approach can include four characteristics.

1. The health care system and health care method of the present example embodiment primarily empower and enable the HCC affiliated to the system with respect to HCPs and also other players and stakeholders in the health care industry (step 1); the health care system of the example embodiment improves cooperation and control (step 2); finally the said health care system creates the basis for coping with a disease, in this case of coping with diabetes and adaptation of lifestyle (C+A; phase 3).

2. Being provided the possibility to communicate with each other within the HCC subset of individuals, the HCC can exchange empirical evaluating information about HCP, information about disease management and treatment results including medication/pharmaceuticals and health care support devices, but also about empirical results of treatment options, support groups and medical therapy groups.

3. The effect of this direct personal information exchange combined with the accumulated knowledge/intelligence of the system providing HCC-specific or HCC category-specific action parameter values to HCP and HCC and combined with the competition mechanisms within the HCP subset of individuals and within the HCC subset of individuals, as discussed above, generates a powerful evolutionary mechanism towards best practices which is transparent and traceable along throughout its evolution.

4. This empowerment of health care clients/patients who used to play the part of mostly passive objects within existing health care systems will promote these health care clients/patients to more active subjects and partners in the collaborative care system within the health care system of the present example embodiment, creating a synergistic unit of HCC and HCP.

In the following detailed description, elements of an individualized disease management of the present technology system and method, with respect to the exemplary diabetes care, are described as a special case of the IHM System.

The present technology system can be a lifelong health management hybrid system consisting of three subsystems; starting from the beginning of life with IHM through individualized (chronic) disease management (IDM) until (lifelong) individualized support management (ISM).

An exemplary actual situation can include a systemic background description of existing problems as challenges.

As discussed above, there is an alienation from individual health management, resulting in a fragmented health care system, which the present technology overcomes. Therefore, a comprehensive and integrative person/patient-centered health care model is needed (see the B-P-P-S model).

We take diabetes as an example that the behavior-related and self-care-related diseases like especially diabetes mellitus type 2 reflect not only an individual's biomedical status (the peak of the iceberg: module 1/layer 1=Bio Marker), but also the underlying psychological situation of the person concerned (module 2/layer 2=Psycho Marker), the behavior patterns and personality traits (module 3/layer 3=Perso Marker), and the basic socio-economic origin of the person concerned with his/her genetic background (module 4/layer 4=Socio Marker): the B-P-P-S model. Health education is not dealt with in elementary, secondary or high schools—nor in colleges or at universities. Although it is the most valuable good of mankind, it is not treated and protected as such. The average citizen in the so-called Trias' of the first world is actually disowned.

An analysis of the role concepts of patients and doctors/HCP's. The research in Europe (in Germany) which also reflects results in the USA and Japan (although the frequency in the groups is certainly different in these countries and the social background influences the results so that in each country a specific analysis is needed) is described in the following in order to give some basic insight.

It has been shown that biomedical treatment (level 1=Bio Marker in the present BPPS model) should not only include the psychological state of the person (level 2=Psycho Marker in the BPPS model), but also the personality and personality traits (level 3=Perso Marker in the BPPS model), and the social origin as well as the socio-economic situation and the social environment of the patient (level 4=Socio Marker in the BPPS model).

This is shown in the following pattern of patients:
Group 1 DETERMINISTIC GROUP: Health is determined by fate (good or back luck).
Group 2 MEDICAL BELIEVER GROUP: I cannot do anything. My (high quality) doctor is in charge of my health.
Group 3 NATURE GROUP: Avoid the doctor and the medical institutions. Live healthy, and everything will be fine.
Group 4 ENLIGHTENED COLLABORATIVE CARE GROUP: I am aware of the fact that it is my health and my life: So I am looking for a doctor/HCP as a professional partner and act as a more or less self-conscious and responsible partner of my doctor and/or the health care professionals.

The following is a description of the corresponding challenges and solutions for the existing problems. The first deals with standardize treatment.

At least one problem being the health care repair systems of today (with the rushed doctor in a fragmented system) are disease-focused with patients as (more or less) an object of a (more or less) standardized treatment.

At least one solution being integrative health care systems with patients as a subject (emancipated as client).

The second deals with separation from the own health. At least one problem being the modern patients are more or less separated from or alienated by their own health; only very few (less than 10% of the population) are really fully empowered and ‘in charge of their individual health management’.

At least one solution being offering low-cost access to empowering and enabling devices for self-care, creating systems supporting the synergy between client and doctor (the Synergistic Unit of Health Care) and thereby creating the desired situation of collaborative care.

The third deals with a need for help. At least one problem being both, patients and doctors, need help.

Let us take the example of the US American society: More than 50% of the doctors suffer from burnout syndrome and doctors starting show the normal depression rate of the population (4%) which increases after one year up to striking 25%.

Let us take the following examples of diabetes care: Only 7% of the US patients reach the three objectives which are relevant to preserve their lives: reaching the blood pressure goals, reaching the objectives for lipids/cholesterol, and reaching the average level HbA_{1c} for blood sugar, avoiding extreme hypoglycemic and hyperglycemic states.

At least one solution being offering a (1) Self-Assessment’ perception of the patient about his situation, (2) Reality Check (lab results and diagnosis), (3) Collaboration Care Action: three key criteria (blood pressure, cholesterol/lipids, blood glucose). This system is of significant help for the patient.

The fourth deals with, in the exemplary, standardized vs. individualized treatment of diabetes type 2 patients. At least one solution being all diabetes type 2 patients are certainly checked in terms of bio-medical status (level 1). This is, however, only the Peak of the Iceberg.

At least one solution being in order to understand the patient's situation and to change it, to have better results, all four layers of the B-P-P-S model need to be considered:
Layer II: Psycho Marker=psychological status of the patient;
Layer III: Perso Marker=personality traits, personal style of interaction and communication, individualized needs for support and individualized support and guidance;
Layer IV: Socio Marker=Socio-Economic Background, for instance the Tipping Points in a social environment, where the patient for instance does not want to be an outsider and stays with the unhealthy lifestyle of his environment.

This is done by the B-P-P-S model, including all four layers.

The fifth deals with treatment of the patient as an object in a standardized procedure. At least one problem being if the patient is treated as an object in a standardized treatment procedure, the results are inferior (especially in person- and psychology-related chronic diseases). As discussed above regarding Examples 1, 2 and 3.

At least one solution being a categorization of patients is needed which is aimed at low costs and with the patient as a collaborative client/subject, and not against the patient's will as an object to be changed.

Group II: collaboration and control [example Diabetes Mellitus Management of three key criteria: (1) blood pressure, (2) cholesterol/lipids, (3) blood glucose].

Another deals with the patient as an object within a highly complex technological process. At least one problem being the cost-driven medical care and health care systems of today have the effect that the patients have become more and more an object within a highly complex technological process. The very disappointing results with chronic diseases and with all diseases which need to take into account the needs of the person show that there is a definite need for change.

At least one solution being empower and enable the patient, enable the patient to be in charge of his health and be in the driver's seat of his health management.
BPPS Model: Group I: Empower and Enable the patient (E & E)
BPPS Model Group II: Collaboration and Control (C & C)
BPPS Model Group III: Coping with Diabetes and Adaptation of Lifestyle and Coping (C & A)

Still another deals with the threshold between patients and doctors. At least one problem being there is a threshold and barrier between many patients and doctors, which needs to be overcome. This, however, is very difficult especially for the complex topics and needs of treating chronic diseases and treating diseases with intimate personal aspects, which require to understand the psychology and the personal situation of a patient in order to empower him to be a client.

At least one solution being support the doctor through a self-analysis and self-assessment of the patient which is giving him access to the inner situation of the patient while the patient still is in a situation to initiate and to control what is going on so that he owns the process. Openness and trust are the basis for collaborative care.

Even still another deals with rational appeals or logic are not helpful. At least one problem being lifestyle adaption and behavior modification for diabetes type 2 patients as well as for patients with depression or the combination of both, patients with depression as well as support for patients with diabetes type 1 (psychological treatment support) is not achieved by rational appeals or logic.

At least one solution being the patient should feel perceived as a person. The person should feel understood and the three steps of the client-centered and non-directive therapy with worldwide acceptance as the basis for individual treatment including psychological and personality issues, (1) starts with unconditioned acceptance, empathy, and understanding and (2) leads to enabling the patients to feel ‘I am okay’ and to be treated as a partner and an ‘equal’. This is the gateway to collaborative care, which (3) finally leads to a collaborative care of the synergistic unit patient-doctor dealing with reality, facing the problems, discussing openly and coming to collaborative care decisions for action.

Yet still another deals with coping with crisis situations. At least one problem being all patients with chronic diseases facing (for depressive patients twice in a lifespan) a crisis where they need definite and urgent support. Leaving patients with chronic diseases alone for themselves does not lead to best results.

At least one solution being there should be below-cost and self-initiated system or device which allows patients to define (step 1) by a Self-Assessment (step 2) with the help of an expert in collaborative care, a medical doctor or health care professional, discussing the lab-results and diagnosis what can be best done for this individual patient (step 3: Action Plan) in order to cope with his chronic disease and to achieve the best possible results.

The 3-step-model of collaborative care, can include:
a) secured and supported/induced by a web-based (low cost) system,
b) patient-driven (patient-initiative=identification=ownership=better results),
c) creating the synergistic unit of collaborative care.

The present technology provides a solution reached by a hybrid system of individualized health management (IHM-IDM-ISM).

In an embodiment of the present technology:
(I) IHM=Individualized Health Management;
(II) IDM=Individualized Disease Management; and
(III) ISM=Individualized Support Management
   are provided using an integrative and comprehensive person/patient-centered automated and web-based health care system integrating IHM, IDM and ISM over the full lifespan of an individual.

In another embodiment present technology: Bio Medical, Psycho, Perso and Socio markers of the BPPS model are integrated into an individual comprehensive Health & Disease Management Profile:

B=Bio-medical (Bio Markers) elements and components (as Module 1/Layer 1);

P=Psychological (Psycho Markers) elements and components (as Module 2/Layer 2)
dynamic criteria as an element or component of bio-medical diagnosis; access to Inner State with intimate personal/patient information through self-report/self-assessment;
the ‘Individual Psycho System’;

P=Personal (Perso-Markers) elements and components (as Module 3/Layer 3);
Partially state of the art as elements of a diagnostic profile;
novelty: the ‘Individual Personal Profile’ as system;

S=Social (Socio Markers) elements and components (as Module 4/Layer 4);
Novelty: the ‘Individual Social and Socio-Economic Profile’ as system.

In another embodiment of the present technology, a unique 5-stage individualized health care/disease management system is provided with:

Stage 1: Self-Assessment (Self-Report) with instant scoring for the resulting (intimate) automated report for the respective person/patient;

Stage 2: Complemented by biomedical facts and diagnostic results (the so-called Reality Check) to a feedback loop: Self-assessment vs. bio-medical facts as learning model for the person's/patient's expertise in health/disease management.

Stage 3: Hybrid Categorization of the persons/patients by using as sources:
(1) Self-Assessment/Self-Report,
(2) Doctor's/HCP's expert rating,
(3) linked to bio-medical and to growing, newly installed integrative BPPS-databanks, creating a hybrid categorization in three dimensions;

Stage 4: (Derived from Stage 3): Individualized Treatment Scheme;

Stage 5: Derived (from Stage 3): Individualized Support Program;

The following three phases of diabetes management are involved and which can include the 10 success factors:
Phase I: Empowerment & Enabling (E & E);
Phase II: Collaboration & Control (C & C);
Phase III: Coping & Adaptation (C & A).

Phase I (Heart) can include the following success factors (SF):
SF 1—Support by family and positive social environment;
SF2—Acceptance of support and guidance from HCP team;
SF3—Positive energy and maintaining motivation; and
SF4—Knowledge of successful coping.

Phase II (Hand) can include the following success factors:
SF5—Open and trustful communication about diabetes problems;
   that lead to the synergistic tandem patient and doctor/HCP, and
SF6—understanding and trust building with doctor and HCP team.

Phase III (Head) can include the following success factors (SF):
SF7—Focus on improvement needs;
SF8—Individually supporting doctor and HCP team;
SF9—Adaption of lifestyle and quality of self-care; and
SF10—Healthy lifestyle and coping (IAP).

In essence, a patient utilizing the present technology would start with Phase I, going through its success factor stages. Proceed to Phase II, which controls the patient's condition, and then to Phase III, which provides success for a final outcome and better quality of life.

In yet another embodiment of the present technology, a web-based treatment support and behavior modification system is provided, integrating three stages to an automated.

The collaborative care system of the present technology can include the following stages and steps.

Stage 1: self-assessment (individual initiative and openness for feedback and learning);

Stage 2: reality check (lab results and assessment by the medical team);

Stage 3: resulting patient-doctor interaction as synergistic unit in the sense of collaborative care:
(1) patient-driven automated/web-based=easily accessible self-care AND collaborative care system;
(2) Supporting both patient (‘person’) and doctor/HCP (Health Care Coach) to realize a synergistic unit with best use of (outcome-related) resources.

In the exemplary, a description of one use of the present technology in diabetes management will be described.

The following description illustrates how a person with diabetes (PwD) and his/her health care provider would use the system of the example embodiment. In this embodiment, the HCP's office (e.g. the medical technician or office manager, depending on the office organization) selects the PwD for participating in using the system:

### Step (1)

In a first step, the HCP's office assigns a reference ID to the PwD and sends this ID as well as the PwD's mailing address to the Service Center. It is assumed here that the PwD is notified by the HCP's office that the HCP would like the PwD to participate.

Upon receipt of the PwD's information from the HCP's office, the Service Center personnel enters the information into a database for future reference.

### Step (2)

In a second step, the Service Center sends a questionnaire and instructions to the PwD's address with a self-addressed, postage-paid envelope. The instructions contain also a letter from the HCP to the PwD with the renewed request for participation.

### Step (3)

In a third step, the PwD answers the multiple choice questions of the questionnaire. The instructional material provides a clear description of this task. In case the PwD needs additional support in filling out the questionnaire, the PwD can access a help desk through calling a toll-free number also provided with the material. The PwD puts the completed questionnaire into the self-addressed postage-paid envelope and returns it to the Service Center.

### Step (4)

In a fourth step, the Service Center prepares the completed questionnaire for electronic processing. This usually includes scanning in the questionnaire to translate the answers into electronic form, assigning the information to the PwD's ID, and storing the information in the PwD's database record. The Service Center performs the actual processing. It sends the PwD reports and the HCP report and the self-care domain questionnaire to the PwD. It also sends a note of completion to the HCP's office stating that the PwD's information has been processed and that the reports have been sent to the PwD.

### Step (5)

In a fifth step, the PwD reviews the report and indicates if there are any areas where he/she doesn't see himself/herself adequately described by the electronically created report. Any conceivable mismatches will be discussed between PwD and HCP during the next consultation. The PwD fills out a questionnaire to assess the current self-care domain status additionally, the PwD is urged to write down questions to ask the HCP and topics to discuss with the HCP at the next office visit. The PwD then sends the questionnaire back to the Service Center utilizing the self-addressed postage-paid envelope.

### Step (6)

In a sixth step, the Service Center processes the answers to the self-care domain status questionnaire. Similar to step four, the preparation consists of scanning and storing the information in the PwD's database record. During the actual processing, the Service Center combines the PwD's information from step four with the self-care domain information and creates the HCP prompt sheet and the PwD prompt sheet. The Service Center sends both sheets to the PwD for use in the upcoming consultation with the HCP. The PwD takes the HCP and the PwD prompt sheets to the next consultation. The PwD should be aware of all information that the system provides to the HCP. If the PwD does not feel comfortable with providing the information to the HCP, the PwD can opt out of doing so. However, this case seems to be very unlikely.

### Step (7)

In a seventh step, the actual meeting between PwD and HCP takes place. The PwD hands both prompt sheets to the HCP. The HCP prompt sheet provides a ‘snapshot’ of the PwD as a person to the HCP including insights into the PwD's preferred interpersonal style psycho-social environment. It also relates in a concise form how the PwD would like to be supported and guided by the physician in case the primary communication concept—derived from the style profile (IDEA) tool—does not prove to be effective with the PwD. The HCP prompt sheet contains the ‘dos and don'ts’ for interacting with the PwD. Preferably, a PwD example dialogue is provided to help the HCP through an initial phase of unfamiliarity with the approach and as occasional refresher during routine use. The HCP uses this information to adjust his/her way of communication to the interpersonal preferences of the PwD. The assessment results of the self-care domain status questionnaire can be utilized to structure the topics discussed during the consultation since the HCP sees directly how the PwD's health care status looks in the PwD's self-assessment, what the PwD considers to be significant, and where the PwD is willing to change. The PwD prompt sheet also contains the topics that the PwD would like to discuss with the HCP during the consultation. The PwD prompt sheet provides space for the HCP to document pertinent lab or physiological results (e.g. HbA_{1C}, triglycerides, etc.) and biomedical or physical facts (e.g., blood pressure, weight) for the PwD.

Reality deviance predictor values (or success factors or need for action levels) can be determined by comparing an extent of a deviation between results of the PwD's self-assessment (subjective evaluation) compared to results of the laboratory tests and biomedical facts on the part of the HCP (objective evaluation). For example, in the self-assessment the PwD answers questions on a questionnaire pertaining to one or more of this patient's biological/medical (physiological) condition, psychological (mental and emotional) condition, personality and genetic factors, and social and socio-economic factors (groups of state parameters). This can correspond to the following reality deviance predictor values or success factors/indicators in which the extent of the deviation between the subjective and objective evaluations is one or more of; extreme difference; definite difference, some difference; and little or no difference, respectively.

The HCP can inform the PwD of the reality deviance predictor values (success factors) as to the level of urgency in a need for action or probability of success in treating the disease or the health problem of the PwD based on the PwD's current self-assessment. For example, the PwD and the HCP have a discussion comparing the self-assessment (how the PwD subjectively views their condition) to the physiological test results conducted on the part of the HCP and the biomedical facts about the condition of the PwD obtained by the HCP (how the HCP objectively views the condition of the PwD). This provides the PwD with a learning model in self-health and disease management.

The HCP writes action agreements and the goals that the HCP and the PwD have jointly agreed upon in the space designed for this purpose. That is, the reality deviance predictor values or success factors can be used by the HCP to determine appropriate action parameter groups including an individualized and collaborative health care action plan for the PwD. The PwD is brought into the decision making process as they see that their mindset toward their lifestyle and how they view the disease (such as overeating/no exercise and for diabetes Type 2 patients this is a genetic disease and there is nothing I can do about it) is compared to an objective evaluation by the HCP. This decision making process may even consider data from other PwDs as discussed below in connection with adaptive or learning nature of the computerized system, such as likelihood of stroke, cardiovascular disease or death, or projected years of reduced life, in PwDs having the same success factors or reality deviance predictor values. The discussion includes the PwD viewing the success factors obtained with the current self-assessment or mindset of the PwD. The individualized and collaborative health care action plan for the PwD can be tailored to reaching blood pressure goals, reaching objectives for lipids/cholesterol, and reaching an average level HbA_{1c} for blood sugar, avoiding extreme hypoglycemic and hyperglycemic states.

### Step (8)

In an eighth step, the PwD follows the recommendations received from the HCP and works on achieving the agreed-upon goals as documented on the PwD prompt sheet.

### Step (9)

A set period of time before the next office visit, the Service Center sends a questionnaire for the assessment of the current self-care status and the topics to be discussed during the upcoming health care visit of the PwD, which constitutes a ninth, step.

### Step (10)

In a tenth step, the PwD fills out the questionnaire and writes down questions to ask the HCP and topics to discuss with the HCP at the next office visit. The PwD then sends the questionnaire back to the Service Center utilizing the self-addressed postage-paid envelop.

### Step (11)

In an eleventh step, the Service Center processes the answers to the self-care domain status questionnaire. Similar to step four, the preparation consists of scanning and storing the information in the PwD's database record. During the actual processing, the Service Center combines some of the PwD's information from step four, especially the personal support and guidance preferences, with the self-care domain information and the discussion topics and creates the HCP prompt sheet and the PwD prompt sheet. The Service Center sends both sheets to the PwD for use in the upcoming consultation with the HCP.

### Iterative Steps 7-11

The flow described above now returns to step seven. Steps seven through eleven are executed iteratively.

Through the initial in-depth assessment of the PwD's interpersonal style, psycho-social environment, and personal support and guidance preferences, the PwD and HCP get a broad but concise picture of the PwD as a person. This is a significant step towards establishing the PwD as an equal partner in a collaborative healthcare relationship that is individualized to optimize the PwD's healthcare outcomes. The iterative, active involvement of the PwD in preparing the consultation by defining the topics to be discussed with the HCP and reflecting on his/her health care status and behavior is another significant step. Also, the focus on collaborative goal setting and follow-up on these goals contributes, with other steps, to achieving sustainable behavior change. This behavior change is not only change on part of the PwD but also on part of the HCP.

The individualized disease management of the present technology system may be used for predicting patient status with diabetes and for controlling therapeutic success using self-adapting model structures for individual disease/diabetes management.

The of the present technology can comprise a method, system, and computer program related to optimal individualized diabetes management with control of diabetes. The of the present technology can be directed to predict the long-term exposure to hyperglycemia and hypoglycemia, and the long-term and short term risk of severe or moderate hypoglycemia and hypoglycemia in diabetes, based on physiologic readings, like ingested carbohydrates, monitored blood glucose, administered volume of insulin and collected data by a self-monitoring patient-doctor-system for individualized disease management with Bio-Marker (biomedical data), Psycho-Marker (psychological characteristics), Person-Marker (personality and behavioral traits), and Socio-Marker (social environment and socio-economic characteristics). The of the present technology, i.e., method, system, and computer program product, enhances existing home blood glucose and ingested carbohydrate monitoring methods and a new self-assessment and self-control system for the patient by himself and in interaction with his/her doctor/HCP's. The of the present technology can use an intelligent data interpretation component, which is capable to predict both blood glucose levels and periods of hyperglycemia and hypoglycemia, and thus to offer options for the doctor/HCP to optimize the individual diabetes management, making use of all four parameters of the of the present technology, i.e. the Bio-Marker, the Psycho-Marker, the Perso-Marker and the Socio-Marker, and to enhance emerging continuous monitoring devices by the same features. With these predictions, a patient's computer model can be created and the person with diabetes can take steps for an individualized diabetes management. With the offered options for diabetes treatment, this will support patient and doctor/HCP to prevent the adverse consequences associated with hyperglycemia and hypoglycemia.

The present technology can analyze all existing patient-related data (BPPS): biomedical data (B), psychological profile (P), personal and behavioral traits and characteristics (P), and social environment and social portrait (S) of the Analysis-Engagement and Support (AES) System of the present technology.

Based upon this system with all success factors (from empirical research) and the four parameters B-P-P-S, the present technology is a novel support for doctor and diabetes patient by predicting the short-term and long-term blood glucose levels and calculating the insulin concentrations to be administered for an almost constant blood glucose level.

The present technology can include a process system for predicting patient status with diabetes and for controlling therapeutic success using self-adapting model structures for diabetes management (for example Neuronal Network Systems).

The present technology system can relate generally to glycemic control of individuals with diabetes, and more particularly to a computer-based system and method for evaluation of predicting glycosylated hemoglobin (HbA_{1c} and HbA₁)/blood glucose and risk of incurring hyperglycemia and hypoglycemia by the help of an individual patient's model.

Extensive studies, including the Diabetes Control and Complications Trial (DCCT) (see DCCT Research Group: The Effect of Intensive Treatment of Diabetes on the Development and Progression of Long-Term Complications of Insulin-Depend Diabetes Mellitus. New England Journal of Medicine, 329: 987-986, 1993), the Stockholm Diabetes Example embodiment Study (See Reichard P, Phil M: Mortality and Treatment Side Effects During Long-Term Intensified Conventional Insulin Treatment in the Stockholm Diabetes Intervention Study. Diabetes, 43: 313-317, 1994), and the United Kingdom Prospective Diabetes Study (See UK Prospective Diabetes Study Group: Effect of Intensive Blood Glucose Control with Metaform in on Complications in Patients with Type 2 Diabetes (UKPDS 34), Lancet, 352: 837-853, 1998), have reportedly demonstrated that the most effective way to prevent the long term complications of diabetes is by strictly maintaining blood glucose (BG) levels within a normal range using intensive insulin therapy. The contents of the aforementioned studies are incorporated herein by reference.

However, the same studies have also documented some adverse effects of intensive insulin therapy, the most acute of which is the increase risk of frequent severe hypoglycemia (SH), a condition defined as an episode of neuroglycopenia which precludes self-treatment and requires external help for recovery (see DCCT Research Group: Epidemiology of Severe Hypoglycemia in the Diabetes Control and Complications Trail. American Journal of Medicine, 90: 450-459, 1991, and DCCT Research Group: Hypoglycemia in the Diabetes Control and Complications Trial. Diabetes, 46: 271-286, 1997 (the contents of both of which are incorporated herein by reference)). Since SH can result in accidents, coma, and even death, patients and health care providers are discouraged from pursuing intensive therapy. Consequently, hypoglycemia has been identified as a major barrier to improve glycemic control (Cryer P E: Hypoglycemia is the limiting factor in the management of diabetes, Diabetes Metab Res Rev, 15: 42-46, 1999 (the contents of which are incorporated herein by reference)).

Thus, patients with diabetes face a life-long optimization problem of maintaining strict glycemic control without increasing their risk of hypoglycemia. A major challenge related to this problem is the creation of simple and reliable methods that are capable of evaluation both patient's glycemic control and their risk of hypoglycemia and hyperglycemia, and that can be applied in their everyday environments.

It has been well known for more than twenty-five years that glycosylated hemoglobin is a marker for the glycemic control of individuals with diabetes mellitus (type 1 or type 2). Numerous researchers have investigated this relationship and have found that glycosylated hemoglobin generally reflects how the average BG levels fluctuate considerably over time, and it was suggested that the real connection between integrated glucose control and BC would be observed only in patients known to be in stable glucose control over long periods of time.

Early studies of such patients produced an almost deterministic relationship between average BG level in the preceding 5 weeks and HbA_{1c} and this curvilinear associated yield a correlation coefficient of 0.98 (See Aaby Svendsen P—Lauritzen T, Soegard U, Nerup J (1982), Glycosylated Hemoglobin and Steady-State Mean Blood Glucose Concentration in Type I (Insulin-Dependent) Diabetes, Diabetologia, 23, 403-405 (the contents of which are incorporated herein by reference)). In 1993 the DCCT conclude that HbA_{1c} was the ‘logical nominee’ for a gold-standard glycosylated hemoglobin assay, and the DCCT established a linear relationship between the preceding mean BG and HbA_{1c} (see Santiago J V (1993), Lessons from the Diabetes Control and Complications Trail, Diabetes, 42, 1549-1554 (the contents of which are incorporated herein by reference)).

Guidelines were developed indicating that an HbA of 7% corresponds to a mean BG of 8.3 mM (150 mg/dl), an HbA_{1c} of 9% corresponds to a mean BG pf 11.7 mM (210 mg/dl), and a 1% increase in HbA corresponds to an increase in mean BG of 1.7 mM (30 mg/dl). The DCCT also suggested that because measuring the mean BG directly is not practical, one could assess a patient's glycemic control with a single, simple test, namely HbA_{1c}. However, studies clearly demonstrate that HbA_{1c} is not sensitive to hypoglycemia.

Indeed, there is no reliable predictor of a patient's immediate risk of SH from any actual data. The DCCT concluded that only about 8% of future SH could be predicted from known variables such as the history of SH, low BC, and hypoglycemia unawareness. One recent review details the current clinical status of this problem, and provides options for preventing SH, that are available to patients and their health care providers (See Bolii: How to Ameliorate the Problem of Hypoglycemia in Intensive as well as Nonintensive Treatment of Type 1 Diabetes, Diabetes Care, 22, Supplement 2: B43-B52, 1999 (the contents of which are incorporated herein by reference)).

Contemporary home BG monitors provide the means for frequent measurements through self-monitoring of BG (SMBG). The calculation between the data collected by the BG monitors and hypoglycemia/hyperglycemia can be done by a special algorithm in which a kind of general patient's model is included. Otherwise this general model does not permit an individual patient's therapy as specific personal parameters like sex, medical records, nicotine and/or alcohol abuses, weight, personal compliance, etc. can be handled in a personal model only.

Therefore, an individualized patient's model is provided by including individual data in the model, which will be methodically collected and verified over considerable time by the system. By the hand of this model, the BG in the early and far future can be predicted, the short-term and long-term risk of hypoglycemia, resp. the long-term risk of hyperglycemia can be estimated, and advice for an optimal therapy resp. a more congenial lifestyle can be given.

Responding to the need of statistical analyses that take into account the specific form of a patient's personal diabetes model, the inventors developed a method which can be described as followed: Based on physiological data of a patient like his blood glucose, his ingested carbohydrate, divided in the three classes fast, medium and slow carbohydrate, and psychological parameters of his self-control via the DISC/IDEA test (four main personality types: DISC=Driver-Introspective-Supportive-Cooperative or: IDEA=Introspective-Driver-Expressive-Amiable), a (neuronal) net structure, following the principle of the self-organizing-maps, is trained to create a neuro-mental (neuronal) representation of his diverse diabetes states.

Next, these different states are combined to a time trajectory describing varying diabetes behaviors, resp. his diabetes histories, over selectable time windows.

Based on these trajectories, a prediction about the patient's status in the early or more distant future is calculated and a value for an insulin concentration to be administered is calculated via a second neuronal network structure to guarantee a strict maintaining of blood glucose (BG) levels within a normal range to prevent hyperglycemia and hypoglycemia.

In parallel, the neuro-mental model and the appointed therapy following from the prediction of the system is evaluated by the statistical method of the cross-correlation in the context of an evident medical care. Also, the DISC/IDEA-model to fix the psychological parameters of the patient's compliance is evaluated successively. In that way, it will be possible to optimize the therapy and the model of the diabetes patient step by step; also by changing the scheme of the therapy accordingly.

(1) First, according to a special aspect of the example embodiment, there is provided a data analysis method and a computer-based system for the simultaneous evaluation of diabetes patients' behavior and the predictive control of their glycemic states from the routinely collected physiological data blood glucose, ingested carbohydrate, divided in the three classes fast, medium and slow carbohydrate, and psychological parameters of a self-control test done by two neural net structures to prevent hyperglycemia and hypoglycemia.

(2) Second, according to a further special aspect of the example embodiment, there is provided a method, system, and computer program product to provide a predictive, i.e., forward-looking, assessment of a patient's medical, physiological, and psychological state based on data obtained on the patient and based on a rule-based categorization formula of the patient per the innovative psychological model, DISC or IDEA, and, based on this assessment, to derive a therapeutic action agreement, such as, for the short term, administering a certain quantity of insulin and managing the patient in an appropriate, individualized manner. This evaluation is done by a combination of two neuronal net structures. The first of them stores momentary patient system vectors, describing a momentary patient status and combining them to time history sheets, which code a medical record over a selectable time window. Out of these records, a prediction regarding the patient's condition in the near or far future can be deduced. The second neural net structure calculates in parallel an insulin concentration, which has to be administered to guarantee strictly maintaining BG levels within a normal range to prevent hyperglycemia and hypoglycemia in the early or far future.

(3) Third, according to a still further special aspect of the example embodiment, there is provided a method, system, and computer program product to optimize the therapy of the diabetes patient step by step by using the statistical method of the cross-calculation to analyze the diverse physiological and psychological data of the patient versus the result of the therapeutic schemes which have been used.

These three aspects, as well as other aspects discussed throughout this document, can be integrated together to provide continuous information about the glycemic control of an individual with diabetes, and enhanced monitoring of the risk of hyperglycemia and hypoglycemia.

The following is a detailed description of the data processing and categorization of patients by a processing example embodiment of the present technology using the example of an application for individualized diabetes management.

The 10 Success factors of diabetes management have been identified in 6 years of intensive empirical studies. The first study was done in the USA (April 2010) with a sample of n=1000 patients (900 Diabetes Type 2 and 100 Diabetes Type 1). The resulting 10 Success factors concept was confirmed in the German study, based on a sample of n=2356 patients. (see www.indimasurvey.com: Studies IS1, IS2, IS3)

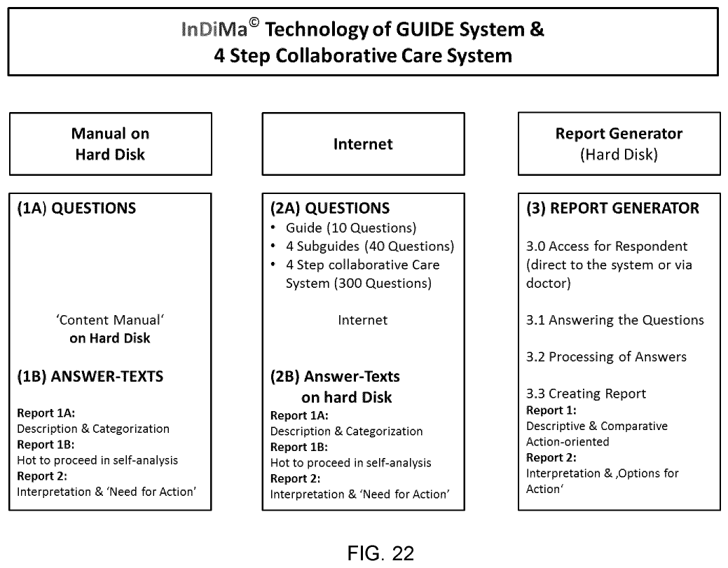

A basic processing system (IBP) of the present technology consists of four program blocks that are integrated into the communication system, with reference to FIG. 22.

The processing system of the IHM and Individual Support Management are identical.

A survey-construction module (to generate questionnaires) can be include that is capable to handle:
a language module (multi-language approach for questionnaires);
a questionnaire construction option to create questionnaires with:
(1) variable scale approaches,
(2) multiple choice possibilities,
(3) ‘open questions’, and
(4) reversed questions;
a questionnaire library, based on items of the communication system, i.e., the 10 core instruments and other parts, which can be added to the communication system.

The survey-construction module is built in such a way that it can export the questionnaires to the web (internet) for data entry and is approachable for the browsers: Internet Explorer (6, 7 and higher), Firefox (2 and 3) and Safari (3, 0). This counts for 98% of the browser market.

Also based on the fact that the exploratory studies where on paper and pencil, the questionnaire export functions are also capable to print the questionnaires into Word documents, HTML-files, ASP files and XML files.

The survey-construction module is constructed in such a way that HCP's/doctors can set-up (after instruction) their specific questionnaires for each patient.

These specific questionnaires for each patient can pinpoint the therapy focus for a specific patient.

The patient-oriented approach of the communication system of the present technology will generate a specific questionnaire for each patient or groups of patients out of the communication system question library.

The present technology can include a report generator tool as an application that works in cooperation with the portfolio system and the survey construction' module.

The report generator is built to create reports for survey trajectories. It is specialized for the creation of personalized reports for surveys.

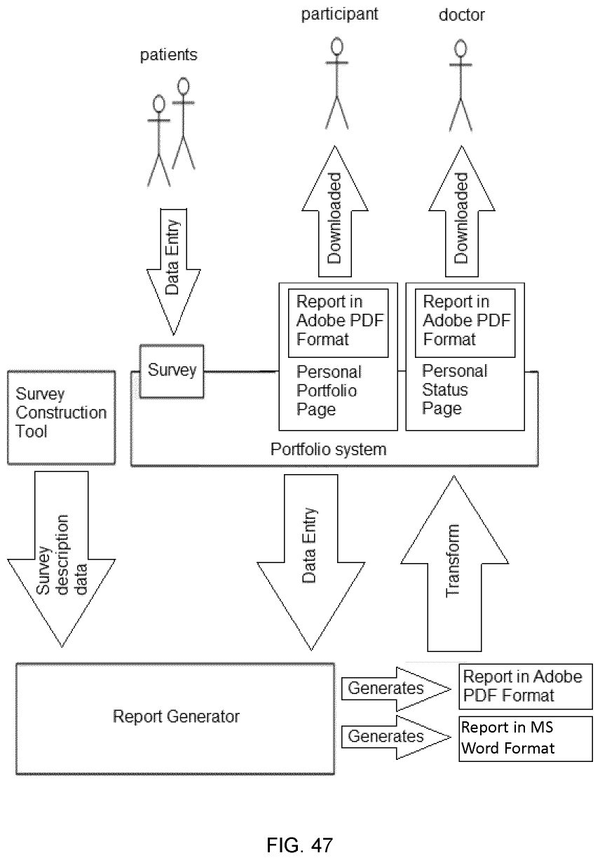

FIG. 47 shows how the system communication between the modules is constituted.

There are four steps to online availability:
Step 1: Data are downloaded to the report generator.
Step 2: Reports are generated.
Step 3: The generated reports are uploaded back to the portfolio system.
Step 4: The reports are available online.

Although some aggregate functions are available, with the report generator tool, a report is always created for a specific patient. The reports thus generated, using the report generator tool, can automatically be uploaded back to the portfolio system so that it is available to both participants (via the Personal Portfolio Page) and doctors/HCP's (through the Portfolio Status Page).

The reports can have structural variations. Usually, every survey can be different from the next. Hence, the resulting reports are likely to vary in structure as well.

That is why the report generator tool can work with templates. Partially complete documents can be re-used and adapted. Hence, they provide the demanded flexibility.

The basis of every report is a report template. A report template is a Microsoft Word XML document and hence can be edited using the widely used and supported Microsoft Word XML.

A report template can contain report template variables. A report template variable can be a part of the report that is replaced by actual survey data, once a report is generated.

There are different types of report template variables. One of them is a reference to a graph template. A graph template shows information (averages, totals) about a specific survey question or a group of survey questions. A graph template is also a Microsoft Word document, but can contain embedded Excel Chart or Microsoft Chart objects. A graph template contains graph template variables. A graph template variable is replaced by a (numerical) value upon report generation.

The SMO/IService Center of the report generator tool can create two types of reports:
(1) individual patient reports,
(2) general reports.

Individual reports are reports that are created for specific patients and HCP's/doctors such as BIP Profiles (Basic Individual Profiles) and Promptsheets.

In the reports, data are used that is linked to a specific patient: The patients and HCP's/doctors are entered in the portfolio system under a Client Management section.

General or Group Reports are reports that use all survey data, for instance for a group of patients for one doctor. Those kinds of reports are used to report on the higher organizational level and not for specific patients but for patient groups or segments of groups.

Based on various settings that can be modified with the help of the user interface, the report generator will insert one or more texts or graphs in a report.

All texts reside in separate Word XML documents that have a particular, pre-set file name.

These documents all reside in a folder, which has a certain, pre-defined structure. Which texts are inserted and where, depends on the place of the variables and the syntaxes used in the report template.

As mentioned before, the report generator tool works in cooperation with a survey-construction module of the present technology. Hence, it is possible to display information from the survey-construction module.

Depending on the settings in the user interface and of course the patient's survey data, a text is inserted in the report. All individual texts are stored in separate Word-XML and Word-docx documents that reside in a pre-defined folder structure.

The following is a description of the portfolio system of the present technology. Data entry can be accomplished via the internet. It also can be used for data entry out of paper and pencil questionnaires.

In the portfolio system, everything revolves around patients. To assure maintainability and to keep large amounts of patients synoptic, the patients are put in a hierarchical group structure belonging to a doctor or a HCP's group or to a Health Care Organization.

Patients are put into the system with a first name, last name, and e-mail address. The patients are grouped and placed into a hierarchical group structure. This structure consists of three levels:
Level 1: Health Care Organization
Level 2: Doctor
Level 3: Patient

Patients normally belong to one doctor in this system.

The combination, however, of diabetologist (level 2) and general practitioner (level 2) might be defined as level 1-tandem (equivalent to a Health Care Organization).

Basically, managing surveys and data entry in the Portfolio system is a matter of two things:
setting up and configuring surveys,
giving participants access to survey by creating ID's.

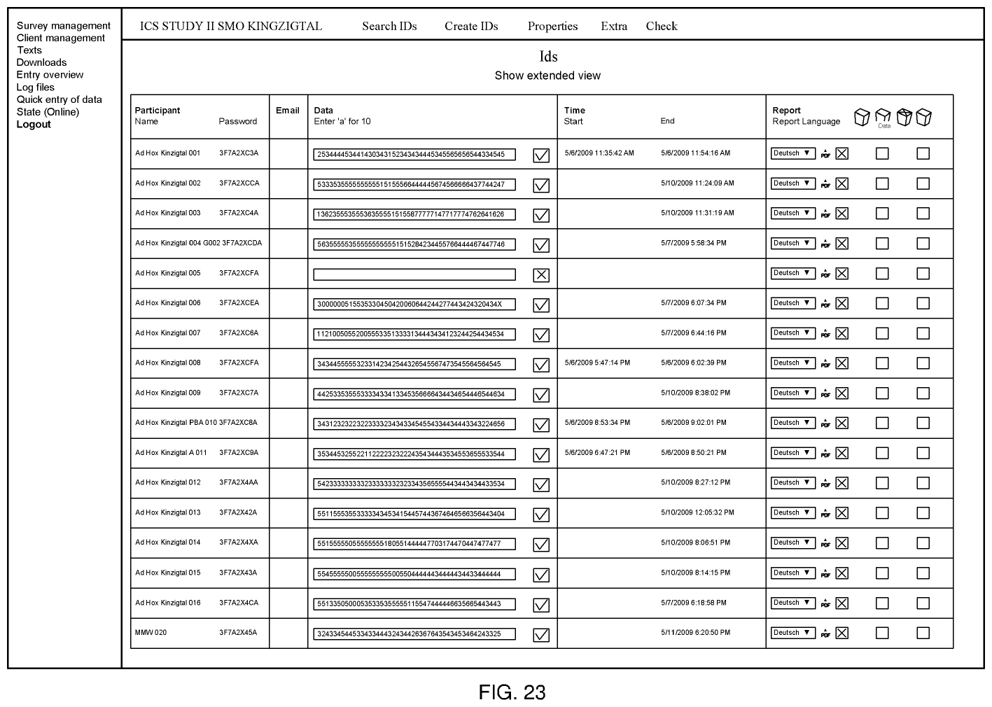

FIG. 23 shows an example of a screen-print of the portfolio system of the present technology.

Participant: In this section the patient's data are displayed. Also the password to access the survey is shown.

Mail: In this column normally the mail addresses of the patients are shown. The icon next to the e-mail address indicates that an instruction mail has been sent to that participant

Data: In the input boxes the data entry of the survey is displayed. Please, note that only the data from input fields, which names starting with question, is shown in this box.

Time: Here, time information about the data entry is displayed. Once a survey is accessed using a password, the start time is set. Once the survey is successfully submitted, the end time is set.

Report: Here the patient report can be downloaded or uploaded.

Trash bins: The checkboxes below the trash bins can be used to delete:
instruction mails for patients. Sometimes the e-mail address entered by the patient is not correct, for example because of spelling errors;
the data entry;
the patient's PDF report;
the complete ID.

The present technology can include a patient/doctor signaling tool.

Patients have a Personal Portfolio Page (PPP) to which they have access by using a password. On the PPP a patient can see if there are any questionnaires to be filled out and can look at the results (reports) of earlier survey processing results.

The doctor can monitor the patient and the group of patients in their own underlying groups. The doctor can ‘login’ to the Portfolio Status Page using an e-mail address and a password. On this page, an overview is given of the patients that are in the system under the doctor's supervision. All patients are shown and reports, linked to the patients, are available on this page.

To maintain contact by e-mail the patient/doctor signaling tool is installed. This means that through this module:
(1) instruction mails will be sent to the patients, with links to questionnaire;
(2) reminders can be sent;
(3) signaling of reports can be sent (also to doctors in the system).

These signals are built as normal templates like a normal e-mail message and are used as the basis for the instruction e-mail. The template contains variables that are replaced by the patient/doctor signaling tool to personalize the e-mail message.

The present technology can include an Innovative Neural Network System Approach for (1) classification (2) individualized treatment and (3) individualized support.

The following is a description of the Neural Network System of the present application for application for patient classification. This can include establishing the present technology model through the present technology system.

The present technology and its software tool serve the purpose of:

(A) building models and processing of extensive sets data for:
the model-based adjustments of neural networks,
the model-evaluation of new models,
the neural-based processing of extensive sets data,
the analysis and determination of linear independence for the analysis and categorization of patients,
all 10 Success factors or within the three groups (E & E, C & C, C & A) from “green” (okay, well developed) to “red” (very critical, urgent need for action), respectively. The application of the present technology system with the integrative BPPS Mode.

(B) The present technology can be used to:
select specific success factors or specific questions, respectively, to be able to check data with regard to biomedical-, psycho-, perso- and socio-markers (BPPS-Modell);
categorize, i.e. to determine the 10 Success factors of the present technology model per patient (10) success factors in diabetes and health management);
categorize specific patients; and
categorize, i.e. to determine groups of patients:
E & E=Empowerment & Enabling
C & C=Cooperation & Control
C & A=Coping & Adaptation [of Lifestyle]

In addition, the present technology has such a wide range of applicability, that it can analyze date from Access-based databases and Excel-based databases.

By means of the tools the available sets of data were used to display the present technology model, empirically elicited by Dr. Martin Muller-Wolf and Dr. med. Wolf-Dietrich Muller-Wolf (see Results from a study in the USA Study (n=1,000): ‘10 Success factors of Diabetes Management’ by Dr. Martin Mueller-Wolf (Medical Affairs: Dr. med. W.-D. Mueller-Wolf); Data Analyses by Prof. Dr. Joan Russo; Medical Quality Assurance and Check by Prof. Dr. Paul Ciechanowski (Apr. 19, 2010)). Three Patient Categories in Diabetes Management (Identified in USA Study n=1,000 and Replicated in a German Study: n=2,356).

Further describing Category I of E & E=Empowerment & Enabling, the four basic factors are summarized with a rather high accuracy, i.e. although these factors are independent, the structures that resulted from the four basic factors were similar to a large extent, so that each factor has to be scrutinized on its own, but that it is feasible and appropriate to gather them in a cluster (no classical arithmetic mean) which does seem to make sense from a medical perspective.

Further describing Category II of C & C=Cooperation & Control, the same applies to category II: Good cooperation and control of the three medical core criteria (in spite of their independence from each other) correlates in practice, too and both aspects form a common frame.

Further describing Category III of C & A=Coping & Adaptation (of Lifestyle), classical factor analysis showed that it is significant to point out that all four basic factors were happening at a high level of patient development in coping with diabetes but that it is also significant to capture very differentiated aspects, namely the following:
Success factor 7: knowledge about self-care and the respective improvement focus;
Success factor 8: Individualized support (not standard-procedures, they are all the same) by the doctor who is treating the patient as an individual and offers individualized support in congruence with the patient's Individualized Support Program (ISP);
Success factor 9: Individualized treatment, considering the quality of self-care of the patient and his overall health status, as well as;
Success factor 10: Coping, adaptation of lifestyle and quality of life, especially for people with diabetes type 1 (genetically-caused diabetes), and the question how the patients cope with diabetes not only physically, but also psychologically (in the sense of quality of life). This applies especially to the age-related diabetes; to cope with the stress of diabetes management, the adaptation to the diabetes reality and its limitations is a challenge for all persons with diabetes.

It is noted that the description of the 10 success factors before the methodological discussion in order to create a basic understanding of the present technology so that the methods of the system and the meaning of the results can be understood more easily.

The following are the results of the evaluation.
(1) All 10 Success factors are confirmed by the neural network NNS analysis.
(2) All three phases of development (Group I, II and III) that have been identified in the empirical studies (n=1000 in the USA and n=2356 in Germany) as so called secondary factors have been confirmed by the NNS analysis:
Group I (E & E=Empowerment & Enabling) with the Success factors 1-4;
Group II (C & C=Cooperation & Control) with the Success factors 5 and 6, as well as;
Group III (C & A=Coping & Adaptation (of Lifestyle)) with the Success factors 7-10 as categories or (initial) patient-categorization for the practical needs of the doctor.

Now it will be described the validated patient categorization corresponding to the factor analysis. Complete content-related correlation of NNS classification and factor analysis classification (see above).

This categorization can also, just like with classical statistic methods, be conducted with a neural Classificator.

The respective categorization leads to a complete concordance of contents and offers additionally a higher grade of differentiation in details while the specific advantage of neural network-systems is that this is done by the self-organizing system without further programming of the software. It was also tested whether this categorization is possible for single patient-questionnaires. The result is a definite confirmation.

One categorization is an individual need for action for each patient. This categorization is shown in the data sets for the 10 success factors of diabetes management through the four categories of Need for Action, as self-assessment of patients and as assessment of the medical team:

[symbol]

“red”=very critical, urgent need for action; [symbol]
“orange”=critical, definite need for action; [symbol]
“yellow”=relatively well developed, some need for action; and/or [symbol]
“green”=okay, well developed.

The results of the NNS-analysis show that the present technology is able to differentiate. The application of the success factor categorization (red, orange, yellow and green) is validated for the categorization of patients concerning Need for Action.

The following is validation of the three patient categories (E & E, C & C, C & A). The NNS-based categorizations showed that the distribution of the patients per success factor and the distribution of the patients per group (E & E, C & C, C & A) are independent from each other.

The result is that the distribution for each success factor and for each phase of development (E & E, C & C, C & A) are validated as categorization, i.e. the NNS-based categorizations lead to the same categorization results as the classical factor analytic method.

The following is more differentiated patient description. As added value of the NNS method it was proven that it leads to a differentiated distribution of persons by describing the individual patterns of patient behavior in a more differentiated manner, using the seven categories of the rainbow spectrum (see www.indimasurvey.com incorporated herein by reference).

The present technology NNS analysis therefore is creating an even more differentiated individual analysis. It allows the prediction of probabilities concerning risks and chances of disease management and the combined cost for health care, based upon the NNS-based categorization.

The NNS classificators of the present technology consist of 10×10 neurons (this is an empirically developed pattern), that have been organized as a closed cluster.

The dimensions of the input vectors for classificators are task-specific, i.e. they depend upon the questions contained in a vector for characteristics (success factors) and the range of their scales (1-5 or 1-10), respectively.

The conditioning of the present technology according to the computing with activities principle will be described. The NNS classificators were, trained in 3,000 learning steps (the 3,000 repetitions are also empirically based steps of conditioning, which were identified as adequate in NNS research (see presentation of present technology in www.indimasurvey.com).

The Gauss-function was used as so called Neighboring Function for the present technology NNS model.

The categorization concepts laid down in the NNS apparatus were evaluated using the principle of Computing with Activities.

To build categories a self-learning rule was used. For the assignment of the colors of the categories need for action a controlled learning rule was used (illustration of expert knowledge).

The constructed tool, the present technology, was developed in C++. No additional software was used.

The tool, the present technology, consists of several surfaces, which allow an individual interpretation of patient categories.

### Step 1

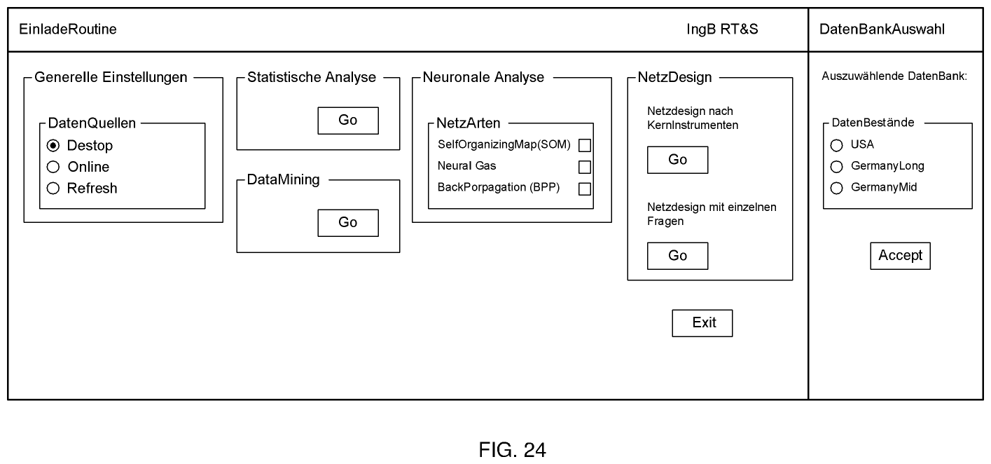

The first surface layer (see FIG. 24) offers a choice for the general modus operandi; in this example, the desktop was chosen, i.e. the data is read from files.

Additionally, this modus operandi allows to choose a database (see right window in FIG. 24) from which the data is to be read.

### Step 2

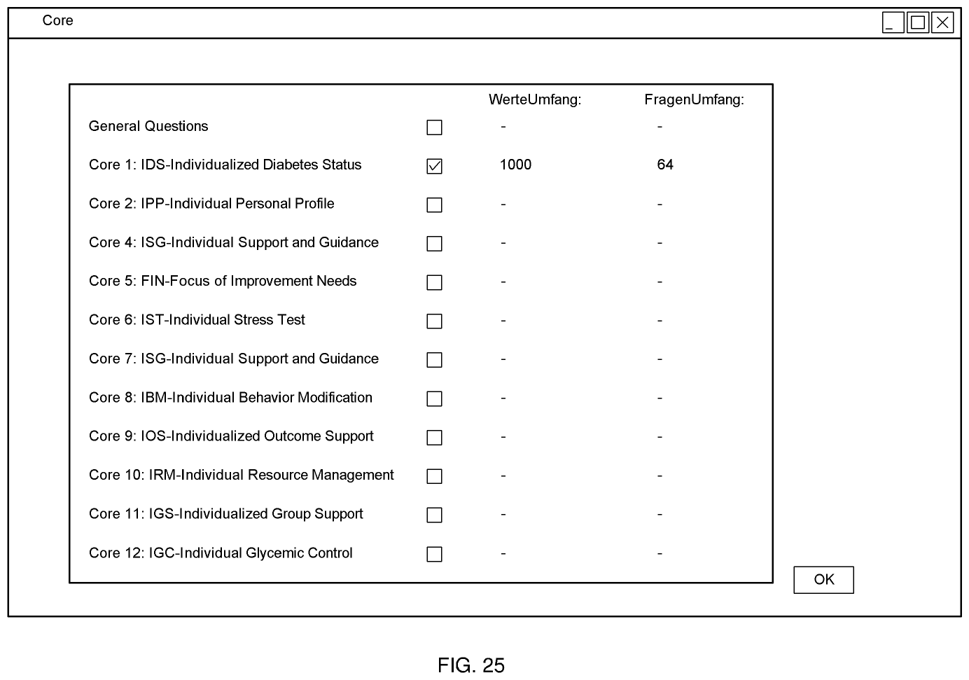

After choosing the database, in this case the database containing the American 10 Success factor Study with n=1000 patients, April 2010, was chosen. The selection of Core Instruments of the present technology is marked by “i” (see FIG. 25: Core Instruments):

### Step 3

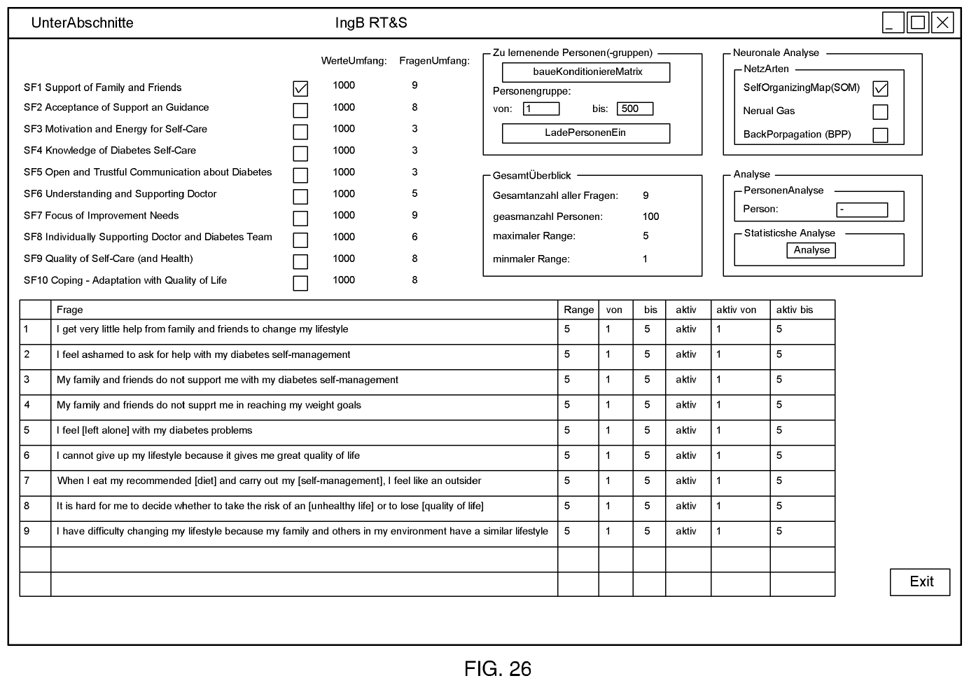

After the Core-choice (choice of instruments) appears working page 3 (see FIG. 26).

The user (patient=HCC or doctor and diabetes team=HCP) can activate the respective Success factors and all the questions they contain. In the example it is done for Success factor 1. In addition, a characteristics vector for 500 persons was activated.

Furthermore, these vectors (questionnaires) have been trained and conditioned. They have learned and have been categorized by means of so called SOM's (Self-Organizing Maps).

This categorization can also be performed as a single persons analysis. For the categorization of the individual patient, a number of persons is inserted into the field in the middle on the right side of the mask shown in FIG. 26.

By pushing the button “Analysis” the person will be categorized with regard to his/her behavior pattern or position, e.g. Need for Action (red, orange, yellow, green) by the NNS.

### Step 4

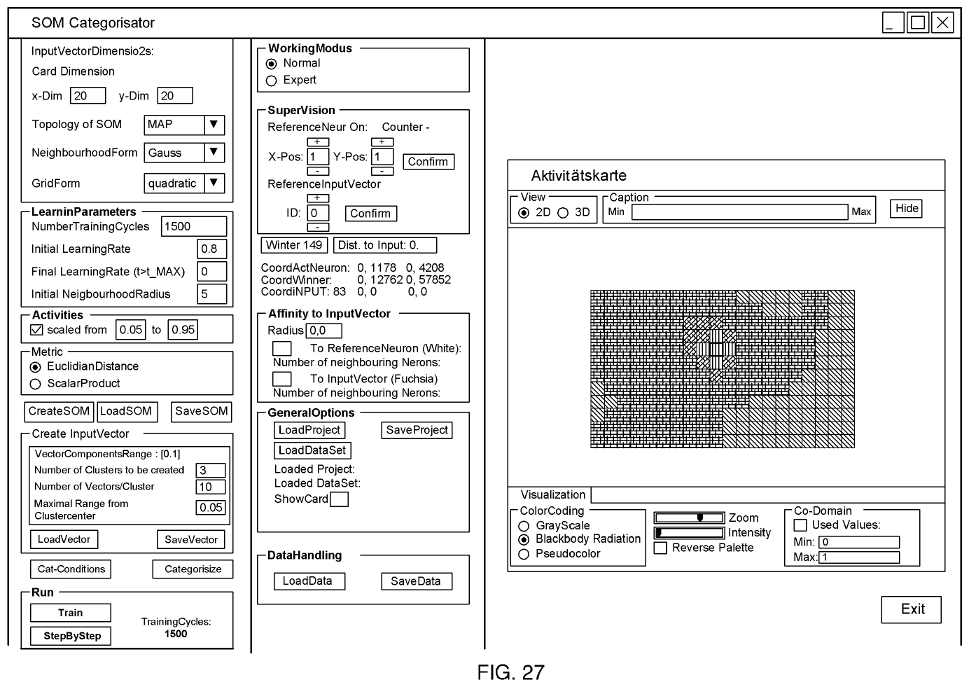

In FIG. 27 the surface of a neuronal classificators (this is the patent-protected adjustment of the model parameter) is shown after its conditioning via Success factor 1.

On the left hand side, one can see the design surface of the neuronalen network. This surface is only visible and usable when the administrator is operating on the Apparatus during the conditioning.

### Step 5

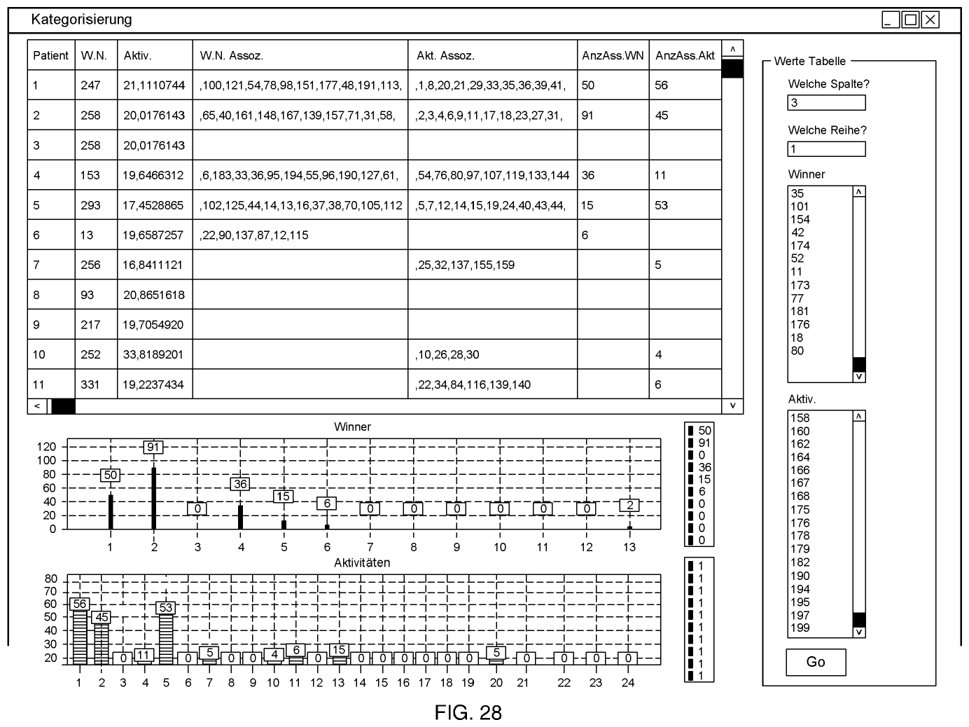

Then the neural net of the present technology does the categorization and shows it. See FIG. 28 for the categorization concerning Success factor 1.

The table in the upper part and the chart below represent the result of a data set of 200 patients. The categories are engaged differently.

In the next step they are assigned to the color scheme of the model, describing the “Need for Action” (red, orange, yellow, green).

From this categorization the HCP can see immediately how many patients are in the different categories or clusters.

FIG. 28 shows 4 main categories or clusters with the number of assorted persons with diabetes: 50, 91, 36, 15.

If the HCP is selecting one patient from the categorization, the program will assign the person directly to one of the categories shown and communicates the result to the user (HCP), categorizing the individual patient.

The following results of the studies on categorization of diabetes patients will be described. The basic research for development of the present technology model according the 10 success factors deals with creating models and the analyses of the different success factor and the three groups according to the empirically detected model.

After the training in the operandi categorization the patient data is analyzed by the neuronal categorizer with regard to:
the number of independent categories of persons that have been found;
the number of categories of persons kategorien that have been identified by the empirically elicited model; and
the evaluation of a model optimization.

The third aspect has to be considered for the learning system in the present technology as a continuous task.

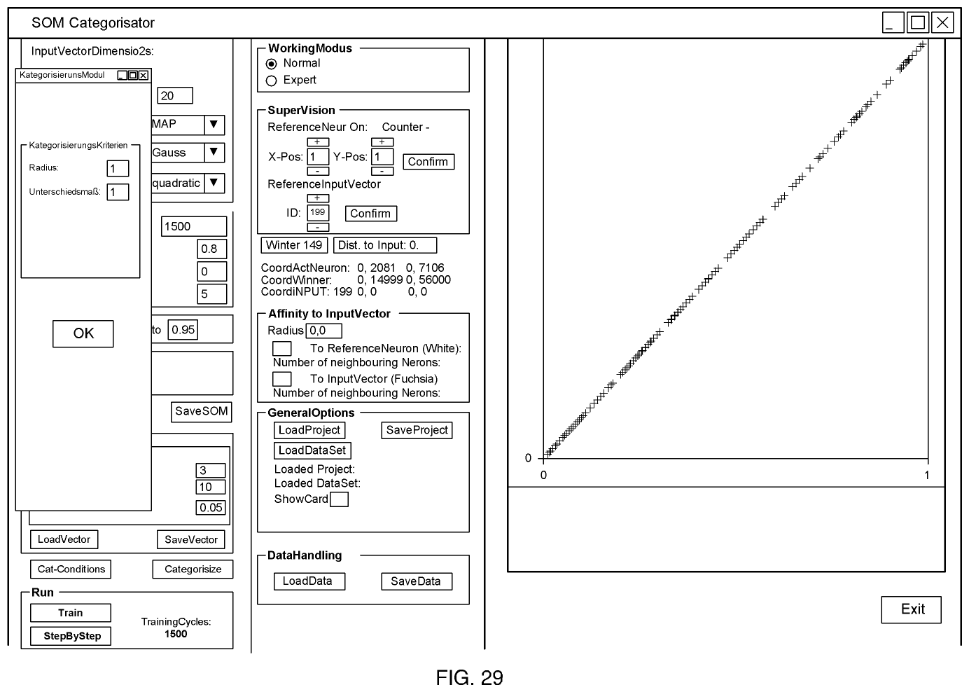

### Step 1: Cat-Conditions as Categorization Criteria of NNS (see FIGS. 29-34)

The key aspect of the work described above is the development of the so-called “Cat-Conditions” (this describes the categorization radius of the neuro-mental analysis of which the model-hypotheses and the expert knowledge of the data form a part).

The basis model of NNS-conditioning was used as a starting point. The adjustment of the categorization radius is shown in FIG. 29. Accordingly, a neuron radius of 7 neurons around each winner neuron and a Cartesian distance of 1 in the activity analysis of the parameter form the best reproduction of the 10 Success factors identified in the US Studies (April 2010) and confirm the present technology model-hypotheses.

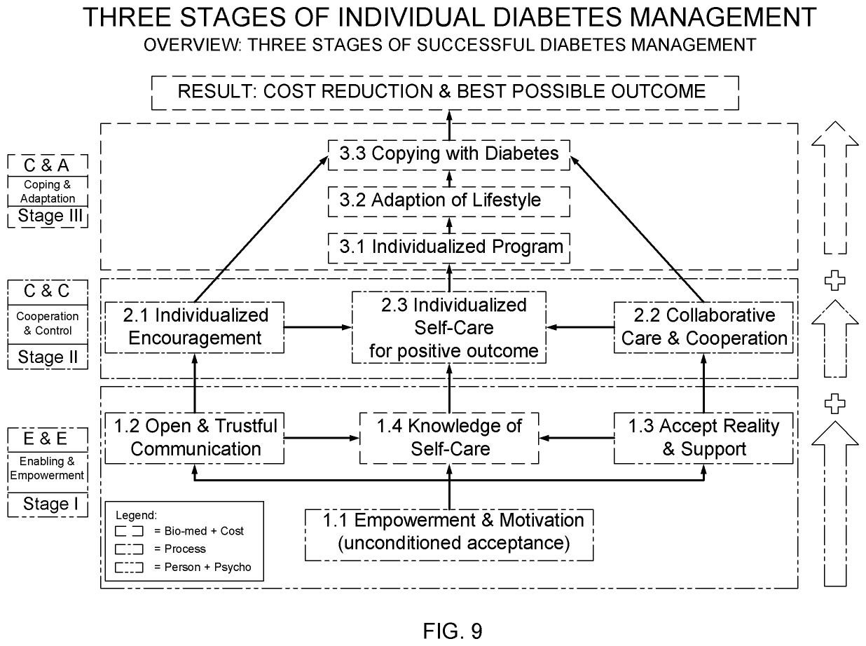

### 6.2 Step 2: Confirmation of the Three Patient Groups (E & E, C & C, C & A) According to the Phases of Development (See FIG. 9)

In the next step the available patient data is analyzed by neural categorizer, i.e. the patient data is scrutinized with regard to being suitable to be assigned to the higher categories.

The evaluation study showed that four categories of need for action (red=very high, urgent need for action, orange=high, definite need for action, yellow=need for optimization, green=okay, no need for action) developed, from which the three patient groups (E & E, C & C and C & A) originated.

The four main categories can be differentiated into 7-12 sub-categories.

The four patient groups according to need for action (red-orange-yellow-green) could easily through adaptation of the color schemes of the model be assigned to the model-based four main categories with the 10 Success factors.

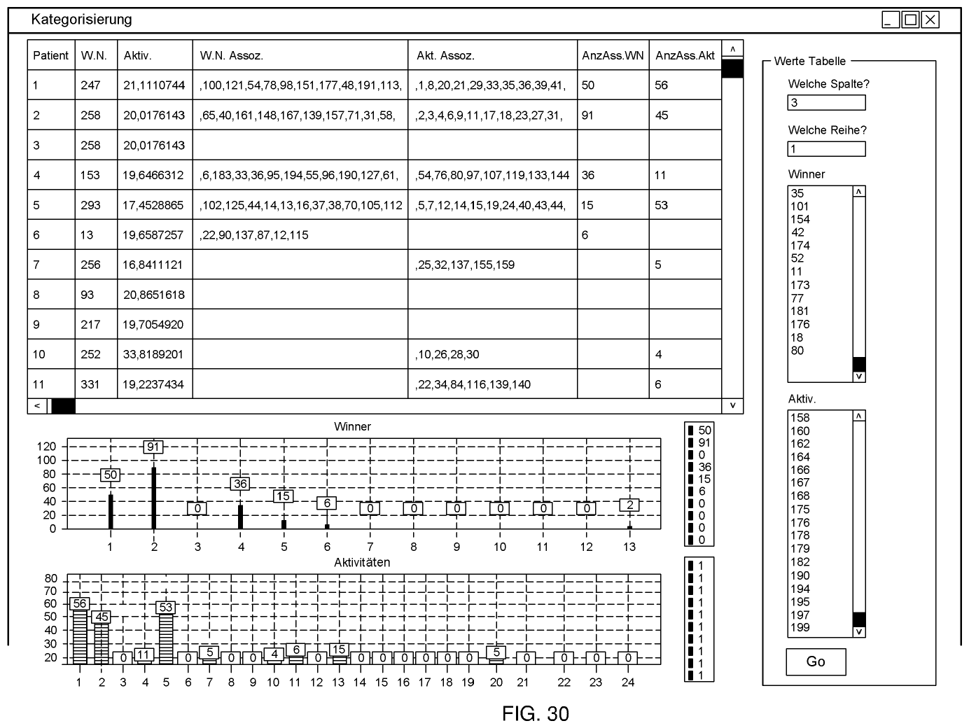

The result of the NNS analysis per se is illustrated in FIG. 30.

In this step each person is compared with all other persons of the sample to create the categorization.

Smaller parameters as shown in FIG. 30 lead to a more sensitive categorization. This is shown in FIG. 30:

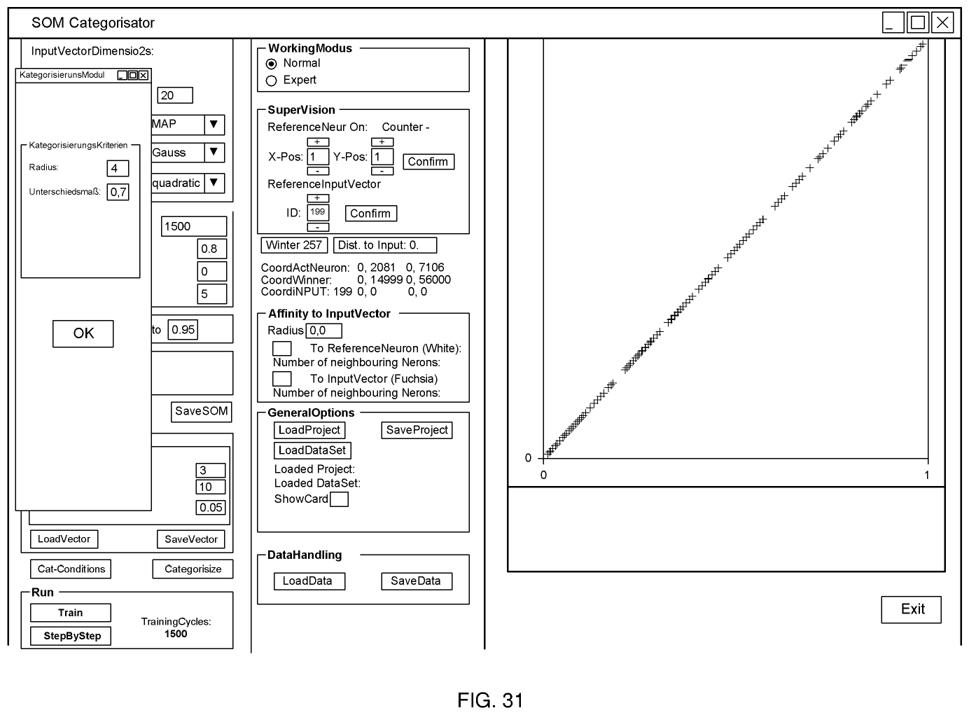

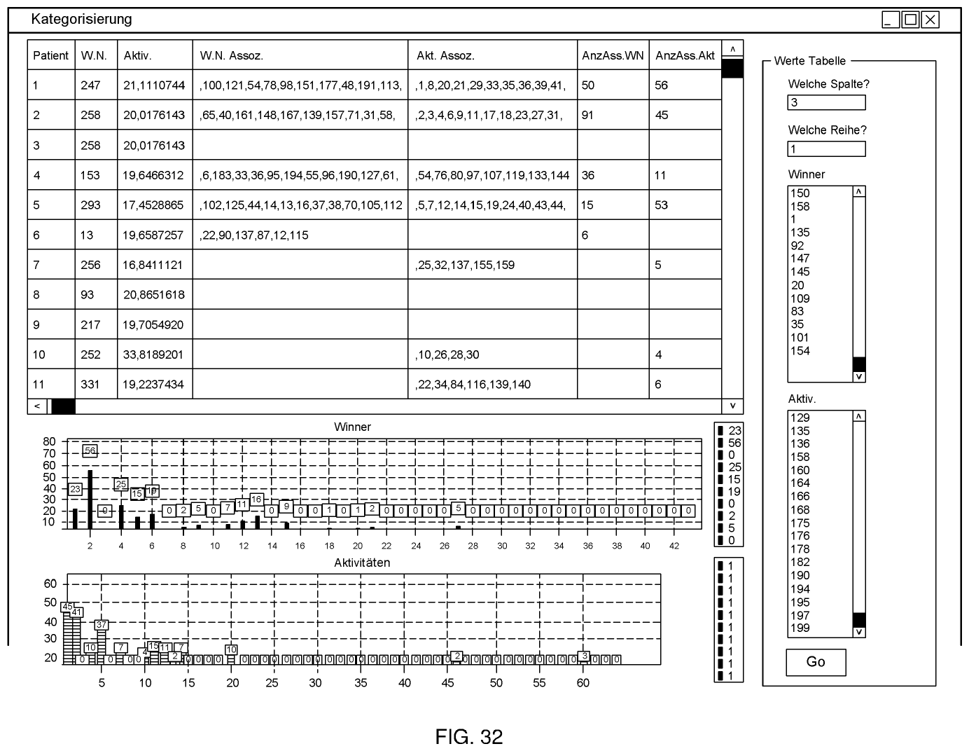

After assigning a person to a group in the first step specific aspects can be investigated in more detail as shown in FIGS. 31-32.

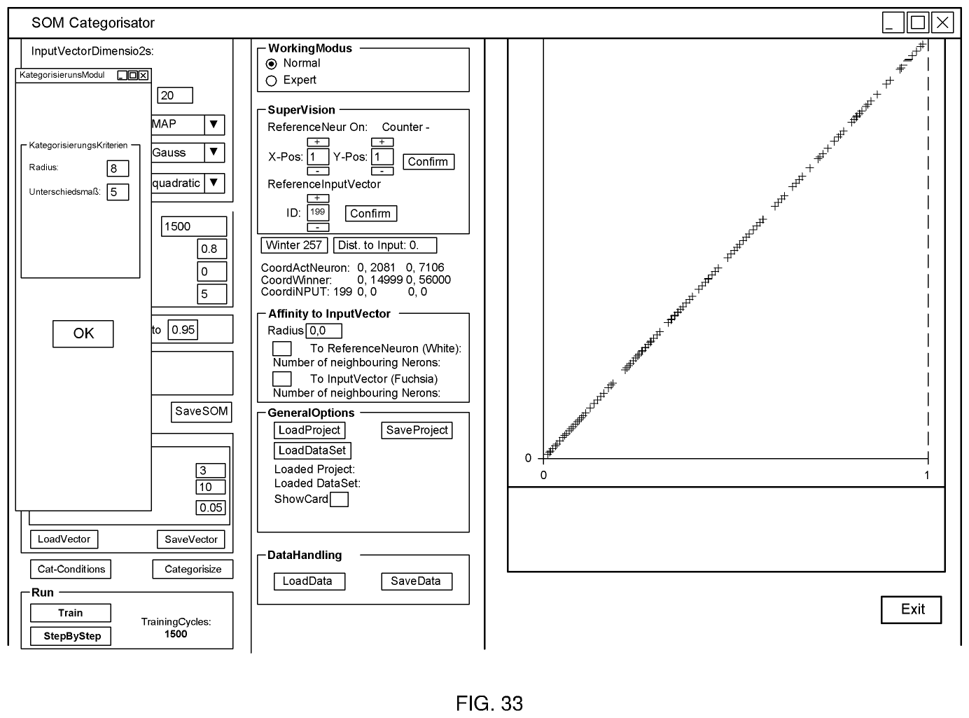

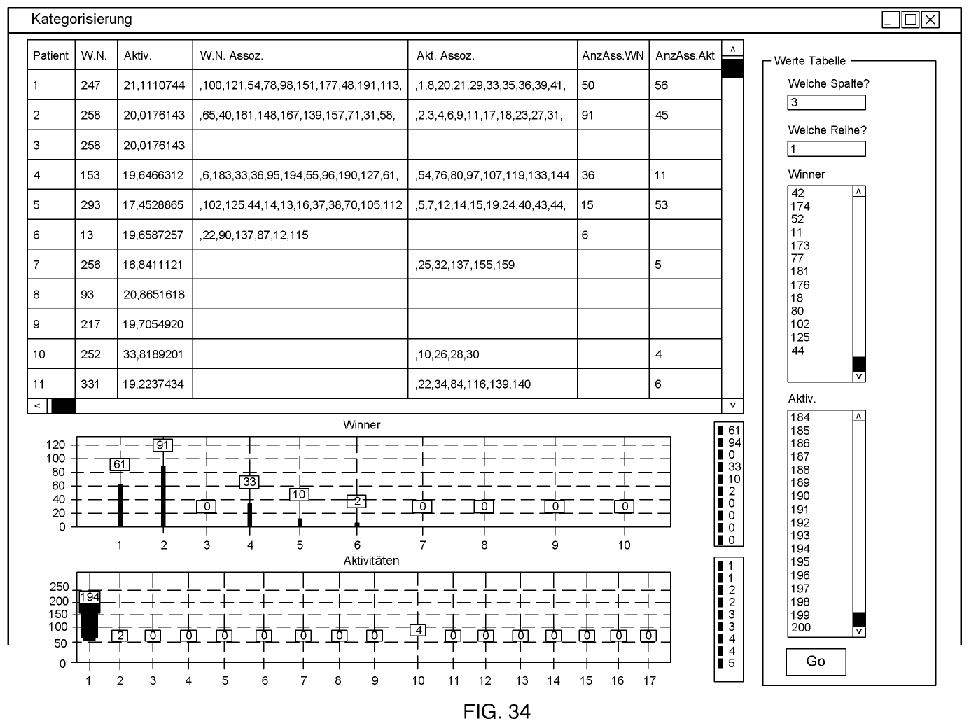

Bigger parameters as shown in FIG. 33 lead to a rougher categorization as shown in FIG. 34.

The present technology can include differentiated in-depth analysis per patient in three steps. In practice, the individual analysis will be significant: We have an individualized system focusing on the single patient.

The group analysis respectively the categorization of patients show its practical value in the field of efficiency for doctors: First of all, a rough pre-selection is made for the doctor in which the overall situation of the patient is shown.

The in-depth analysis can be realized in three steps:

### Step 1: The Overall Situation of the Patient

Display of the overall situation of the patient including all 10 Success factors in a neuronally defined paint dot.

The colors green=okay to red=very critical situation, urgent need for action. As well as the colors in between orange=critical and yellow=to be optimized offer a graphic overview on the overall situation of the patient.

### Step 2: Categorization According to the Three Main Medical Stages

The three main medical categories are according the three stages of diabetes management:

Stage E & E (‘Empowerment & Enabling’): Regularly patients with eating addiction and weight control problems, often with adiposity, who lack motivation (Empowerment) and knowledge about self-therapy (Enabling) and especially where the interrelation of both of them (motivated to actively change their situation and to adapt to the challenges of diabetes management) is problematic.

This can be done in a third step for each success factor by means of an individual analysis.

Before that, in Step 2, a categorization is performed:

Stage C & C (‘Cooperation & Control’) of Success factors 5 and 6 in connection with Empowerment and Enabling by the doctor:

On one hand trust and openness about diabetes management problems and control of the three medical core criteria (blood pressure, cholesterol/lipids, and blood glucose control), on the other hand an understanding and not judging doctor who is taking into account the specific needs of the patient who can motivate (=Empowerment) and actively supports the patient in coping with diabetes (=Enabling).

Stage C & A (‘Coping with Diabetes & Adaptation [of Lifestyle]’): Patients who are able to control to a large extent the medical and psychological problems of diabetes and to adapt their lifestyle according their diabetes.

### Step 3: Individual Analysis

There are four different factors that can be analyzed for each success factor in Step 3:
Success factor 7: Does the patient know about the focus for improvement needs in his self-care?
Success factor 8: Does the doctor (from the patient's point of view) offer an individualized specific medical help and support?
Success factor 9: How good is the quality of self-care and the overall status of health of the patient? Where is potential for optimization in detail and how can it be achieved?
Success factor 10: How is the quality of life, the overall status of health as well as the psychological condition for coping with the diabetes reality in the long run (avoidance of depression and burnout)? This is significant for the genetically determined diabetes type 1 since the necessarily bio-physical and bio-medical adaptation to diabetes is given (otherwise they will die).

The present technology can include a comprehensive NNS analysis and cross-check. If one analyzes every single success factor, one can see that the success factors have a different distribution.

A rough analysis according to the model herein shows the following results, as shown in Table 4:

TABLE 4

| Success | | | | | | | | |
|---|---|---|---|---|---|---|---|---|
| factor | red w | red a | orange w | orange a | yellow w | yellow a | green w | green a |
| 1 USA | — | — | 114 | 154 | 41 | 32 | 42 | 4 |
| 1 Germany | 57 | 91 | 42 | 61 | 92 | 27 | — | — |
| 2 USA | — | — | 62 | 17 | 43 | | 7 | 3 |
| | | | | | (130)* | | | (130)* |
| 2 Germany | — | — | 47 | — | 56 | 81 | 93 | 100 |
| 3 Germany | — | — | 7 | — | 49 | 62 | 131 | 112 |
| 4 USA | 72 | 69 | 72 | 69 | — | 13 | 53 | 40 |
| 4 Germany | 8 | — | 5 | 17 | 9 | 3 | 174 | 158 |

*Mixed class

Table 5 serves as a cross-check:

TABLE 5

| Color | % winner-related | % activity-related |
|---|---|---|
| Red | 0.097 | 0.114 |
| Orange | 0.25 | 0.23 |
| Yellow | 0.25 | 0.36 |
| Green | 0.36 | 0.35 |
| Sum | 0.96 | 1.05 |

The following is a summarizing analysis of categorizations

Confirmation of the four need for action criteria (Red, Orange, Yellow, Green) by the 10 success factors The analysis of each individual Success factor shows as result that all success factors have the same ‘distribution pattern’ (with the categories ‘red’, ‘orange’, ‘yellow’ and ‘green’).

An individual analysis of the 10 success factors can include combining a plurality of success factors the NNS-analysis shows that the factors do not lead to the same creation of categories beyond the 10 success factors.

This means that the individual success factors, as was already proven by the factor analysis, is statistically more or less independent from each other.

The categorization (red, orange, yellow and green) has to be performed for each of the 10 success factors: It cannot be performed by the same categorizer in one step for all 10 success factors.

There can be linear independence of the 10 success factors. There are groups of persons which are displayed differently on the 10 success factors (e.g., success factor 1 ‘green’=high social support, but for Success factor 10 showing ‘red’=not coping with diabetes at all).

Therefore, by the analysis of the neural network system of the present technology, it is proven that the 10 success factors are in the state of linear independence.

This confirms the Need for Action categorization of the model, which was determined by classical factor analysis (see: www-indimasurvey.com: studies IS2 and IS3).

There can be significant and medically relevant results for individualized diabetes management. In above Table 1 the different success factors and their significantly relevant categorizations are displayed:
The size of the significances is related to the size of the sample: Highly relevant significances are identified as ‘statistically relevant’ even if the sample is relatively small.
This is the case for all categorizations of Need for Action, which are significant for all patient groups and for all three stages of diabetes management:
E & E=Empowerment and Enabling;
C & C=Cooperation and Control;
C & A=Coping with Diabetes and Adaptation (of Lifestyle).

An enlargement of the sample led to all categories being proven significant.

The following is a summary of the experimental results associated with the present technology, leading to a confirmation of the four Need for Action criteria of the model.

Both, the winner-related analysis (first generation of NNS-analysis) and the activity-related analysis (third generation of NNS-analysis), confirm the validity of the Need for Action mode.

Four Need for Action categories of the exemplary diabetes management system of the present technology were validated for each one of the 10 success factors

One more category is added which shows that the questionnaires have not been filled in completely by the patients. This is also relevant information, since not filling out the questionnaire is in many cases an indication for a Need for Action category and for a Personal Interview of the patient by the doctor or the medical team.

The following is a description of a Need for Action Sensitivity and Completion Sensitivity’ of the present technology. From this finding and the prior analyses, the two advantages of the chosen neural approach become apparent:

(1) The neural classificator is model-based variable.

This means that the NNS categorization radius can be compared for different models with the same categorizer. This makes an evaluation of the models possible.

This means the NNS-classificator is model-sensitive and can at any given time applicate rougher (need for action vs. no need for action) or more sensitive models (e.g., seven instead of four categories of Need for Action) and test them in comparison.

Classical categorizers can only do this to a certain degree, e.g., new calculations, restructuring or manual improvements.

(2) The neural classificator is completion-sensitive. Classical categorizers can only do this to a certain degree, e.g., by manual improvements.

Concerning the categorization results of the three groups C & C, E & E. and C & A it is remarkable that especially within Group III C & A the four individual Success factors (SF 7, SF 8, SF 9, SF 10) have to be analyzed individually.

This can be explained by the fact that through the non-linearity, i.e., independence of the Success factors whose amalgation in main categories (secondary factors) brings a model-conform result, but, as in well known classical statistical analysis, a differentiated analysis of the four Success factors (SF 7-10) per patient brings a differentiated impression with relevant additional information.

The present technology can include an innovative 3-Step approach. The present technology is ready for practical use of the doctor, the medical team, the administrator in the health care ministry, the manager of a hospital or for the use by the interested patient for his self-analysis and the preparation of an Individualized Action Program:
Step 1: Comprehensive overall picture: Categorization of the patients in Group I (E & E), Group II (C & C) or Group III (‘C &A’);
Step 2: Analysis of the specific selection out of the 10 success factors (per patient or per group); and
Step 3: Differential Analysis per Success factor on item basis for the Individualized Action Program of the patient or the specific patient group.
The following is a detailed description of the present technology example embodiment Example 2: Application for Precise Glycemic Control in Individualized Diabetes Management.

The example embodiment makes it possible, without being limited thereto, to create precise methods for the evaluation of predictive diabetic's glycemic control, and includes firmware and software code to be used in computing the key components of the method. The inventive methods for evaluating BG, the long-term probability of SH, and the short term risk of hyperglycemia and hypoglycemia, are also validated based on extensive data collected, as will be discussed later in this document. Finally, the aspects of these methods can be combined in structured display or matrix form.

Creating a computer based memory of individual patient's medical records can be accomplished by the present technology. Specifically for the example embodiment, a microcomputer system is controlled by a program stored in a permanent memory on the hard disc drive (secondary memory) of a computer in such a manner that the following method steps may be carried out: Controlled by an internal or external clock, binary-encoded sensor and categorization data on a patient are supplied to a microcomputer system, which converts these data to a vector form in a defined sequence. The vector is called the patient status data vector (PSV). The PSV represents the patient to be assessed and treated by his blood glucose, the ingested carbohydrate divided in the three classes fast, medium and slow carbohydrate and the parameters of his self-control test described below.

To create an individual experience-based model of the patient, a sufficiently large number of physiological or psychological states/data for example from the questionnaire have to be integrated in the model using the PSV, with components resulting from the data mentioned here. Furthermore, it may be assumed that a causal relationship exists between a personal system state at time t and a personal system state t+Δt, when Δt is sufficiently small. This means, however, that the denser a stored time history, defined via Δt, is of all patient system states, the more complete the experience-based model of the patient is, and, as a consequence of the causality of the personal system behavior, the more complete the ability of the patient model is to predict future states of the patient and, therefore, to intervene in his physiological state in order to bring it under control. For this to be accomplished, however, all possible patient states and future states based thereon have to be stored in a symmetrical manner done by recording the patient's state vectors over long time and diverse life situations. Also, known patient's situations as hyperglycemia and hypoglycemia states have to be stored in this symmetrical manner, which can be done by simulated PSV also.

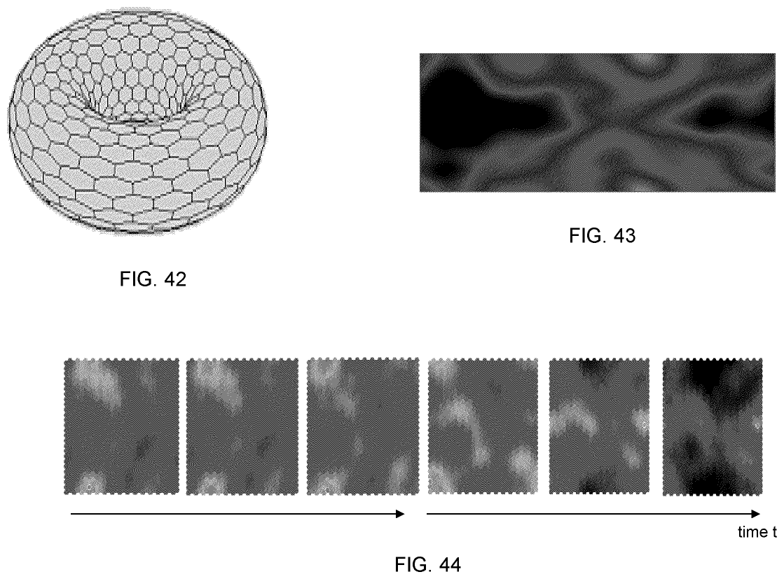

In an inventive refinement of the current technology, an expanded, computer-aided memory model for these large amounts of stored PSV's is used, which serves to combine a combination of computer-aided estimators (neurons) into a neuronal network. These estimators are coded in a way that they are placed on a topologically closed, two-dimensional surface such as the torus-like surface shown in FIG. 42 on a regular or irregular grid formed of the estimators. In a first advantageous embodiment, it is thereby made possible to assign the same number of adjacent estimators to every estimator. This means it is possible to assign the same number of similar system states in a topologically adjacent manner to every personal system state that is categorized by the values of the estimators. Fringe effects and cut-off effects, which are known from the literature, may therefore be avoided, i.e., all of the estimators as a whole may categorize system states in a continual manner (dee Reuter: Computing with Activities, Computational Intelligence, LNCS 2206: 174-184, 2001 (the contents of which are incorporated herein by reference)).

In a further embodiment of the present example embodiment, the actual categorization of system states is not carried out using only one selected estimator, e.g., the estimator with the highest output value, but the activity of all estimates, as shown in FIG. 43. So the activity pattern of the whole torus represents the categorization of a PSV, i.e., the matrix of all estimates represents a system state. Using this kind of categorization, it is possible to encode a larger number of distinguishable categorizations as known before, e.g., using 10 times 10 estimators and a band width of their estimates of 16 bit, 10²⁴ PSV's.

To create the experience-based model of a patient itself, diverse PSV's are presented to the neural net described above in its selectable “conditioning” operating state. These PSV's may represent real patient states recorded over the time or PSV's which have been created in a reasonable, synthetic manner, i.e., simulated or designed on the basic of medical knowledge. During this conditioning phase of the neural net, which is controlled via the mathematical conditioning formula of the SOM's, the structure of the estimators is modified such that the neural net/network formed by them store the presented patient's states in its structure in a way that they may be called up in a defined kind. In detail and common to the theory of the neural nets, this may take place by changing the connection weights between the estimators of a first estimator layer, which represents the PSV components, and the elements of a second estimator layer, which perform the actual categorization. This procedure is known as the self-organizing-principle of simulated neural maps with N estimators, briefly named SOM's. (See book of Kohonen T.: Self-Organizing Maps, 1995 (the contents of which are incorporated herein by reference)) and can be formulated briefly by the formulas, Equation 1.

Δₓ(x^{t}ⱼ,w^{t}ₛ) min{Δₓ(x^{t}ⱼ,w^{t}ₛ)|i=1, . . . , N Equation 1

whereby Δₓ(x^{t}ⱼ,w^{t}ₛ) denotes the difference between the presented PSV x^{t}ⱼ at an given time t out of an ensemble with the serial number j and w^{t}ₛ the weight vector from all estimators of the first layer to the estimator s with the largest activity. Next, the weights of all estimators are adapted regarding the estimator of largest activity by the formula Equation 2

wᵢ^{t+1}=wᵢ^{t}+ε^{t}·hₛᵢ^{t}·Δₓ(x^{t}ⱼ,w^{t}ᵢ) Equation 2

with the time-dependent learning rate ε, Equation 3

$${ɛ}^{t} = {ɛ}_{start} \cdot {( \frac{{ɛ}_{end}}{{ɛ}_{start}} )}^{\frac{t}{{t}_{max}}} \qquad Equation 3$$

the neighborhood function h^{t}ₛᵢ, Equation 4

$${h}_{si}^{t} = {e}^{\frac{- Δ {A ( {k}_{s} , {k}_{i} )}^{2}}{2 {( {δ}^{t} )}^{2}}} \qquad Equation 4$$

and the adoption function δ^{t}, Equation 5

$${δ}^{t} = {δ}_{start} \cdot {( \frac{{δ}_{end}}{{δ}_{start}} )}^{\frac{t}{{t}_{max}}} \qquad Equation 5$$

is used.

The variable “start” denotes the values of the time-dependent parameters at the beginning of the conditioning operation state, “end” the values of the time-dependent parameters at the end of the condition operation state and tₘₐₓ the maximum number of conditioning operation state steps to be calculated.

In this way, all PSV's are coded in the weight structure of the neural net if the conditioning operating state is finished, whereby similar PSV's correspond to similar activity patterns of the net.

Due to the structure of the second estimator layer described, it is possible to store all presented sensor and categorization vectors and therefore, patient states, in constant form in the estimator-based and therefore in an experience-based model and, therefore, due to the adjacency of causally successive patient states, to define a causal chain of events via the second estimator layer using its related modification of its estimators. Due to the mathematical conditioning formula and the large memory capacity, the categorizing neural network may be continually modified without losing its previously-stored structure. As such, it is possible to expand a system model continually, in an adaptable, experience-based manner (See Reuter: Ruling Robots by the Activity Patterns of Hierarchical SOMs, ISC '2003 (Eurosis03), 2003 (the contents of which are incorporated herein by reference)).

Due to the structure of the neural nets in a selectable “classification” operating state, PSV's can be categorized if the neural net is conditioned by some basic vectors or by a complete set of the patient status data vectors, whereby the quality of the result of the categorization depends on the density of the different statuses already coded in the neural net during the condition state. This recall takes place in that way, that an activity pattern of the neural net neurons Φ₍ₜ₎ for a given time t corresponds to one and only one PSV at the same time t and that similar activity patterns Φ′_{(t′)} of the neural net correspond to similar PSV values. In detail in the classification modus of the net, a PSV is presented by the network by presenting the components of the PSV to the first layer of the neural network. Based on its special—learned—structure, the second—classification—layer of the network will be induced to an activity PSVs2 which is similar or equal to the activity PSVs1 which has been stored before in the conditioning state whereby the difference between PSVs2 and PSVs1 is coded in the different activity patterns Φ₍ₜ₎ of the neural net when PSVs2 or PSVs1 is presented to the neural net. This difference will be as larger as larger the difference between PSVs2 and PSVs1, whereby the mathematical dependence of this difference depends on the chosen neighborhood function h^{t}_{Si}. In that way, even PSVs that have not yet been presented, but are assigned to patient states stored in the model to an extent defined in the conditioning formula, will be categorized so that a continuous chance of activity patterns over the time denotes a continuous change of a patient's state over the time t.

In a further advantageous embodiment of the present example embodiment, the activity patterns Φ₍ₜ₎ categorizing the patient's or the patients' states can be decoded by a second kind of neural nets following the backpropagation scheme and displayed on a monitor or announced by a loudhailer. Thereby the backpropagation net is trained in that way, that an activity patterns Φ₍ₜ₎ is assigned to a patient status PS(t). The appropriated backpropagation net consist of three layer of estimators, whereby the first layer—the input layer—gets the activity patterns Φ₍ₜ₎ from the first net in a one to one way, the second layer—the hidden layer—transform the activity pattern in a new neuro-mental representation and the third—the output-layer—represents the probability of the patient's statuses via several estimators, whereby every estimator represents one patient status.

Due to the structure, all calculated and measured values will be stored on a hard disc drive or a removable storage drive for later use.

Due to the structure described in the previous paragraphs, it is possible to categorize every PSV, and a medical report reps. a trend analysis of the patients diabetes status can be calculated by combining several Φ₍ₜ₎, resp. PS(t) in chronological order. The resulting time trajectory of the PS(t) then describes varying diabetes statuses, resp. the diabetes value history over selectable time windows, which can be visualized or explained in an acoustic way. In that way, the patient can control himself resp. his course of disease/therapy.

The present technology can include creating a computer based predictor for BG estimation. Alternatively, the example embodiment provides a method, system and computer program product for predicting one or several patient's physical or psychological parameters via a predictive control module. This predictive control module is constituted by a neural chain of estimators, whose principle hierarchical structure is the same as described above, whereby this chain now is used to calculate the patient states and/or the BG and/or both to be expected in the near or far future, with the help of his individual personal model.

For this propose, the components of the PSV's of a selectable time window T, represented by an independent measured PSV's, are combined in any order to a new (enlarged) personal status vector EPSV. Representing the history of the patient's status in the time-window T, this EPSV forms one of the diverse EPSVs, which are used to create an experience-based model of the patients time behavior in the same line as described above. Also in the same line as described above, this model will be decoded by the second net structure, whereby the output of this structure now will be a predicted value/parameter or directive, for example the BG-value to be expected in the next hours.

Next, on the basis of these predicted values/parameters or directive or machine command, a patient directive or a simple record will be created, whereby due to the structure of the method, system and computer program product, a special interface for an external expert system guarantees that the predicted values/parameter or directive can be adequately transferred to medically sensitive machine commands, patient directives or records.

Due to the structure, the “conditioning” operating state may be activated during on-going operation or during any periods of time for all neural nets and in this, the ascertained nets may be validated by a rule-based optimization algorithm whether they still satisfy a given categorization standard or whether they have to be sensitized, to optimize the categorization system.

As Example of the present technology application for diabetes management, an evaluation of patient's type by self-assessments or external assessments can be accomplished.

In a further advantageous embodiment of the present example embodiment, the patient's medical and physiological status is linked with data that are measurable via electronic means and which were obtained via self-assessment or external assessment (by the patient, physician, or diabetes experts) in the “Patient DISC/IDEA Test” (or in other psychological and psychological tests like present technology). This enables a standardized prognosis of the course of the specific patient's condition, an individualized prognosis that takes into account the patient's pattern of behavior, which was ascertained electronically (psychologically and empirically on a patient typology determined via random sampling).

In a further advantageous embodiment of the present example embodiment, the consultation and patient-care style of the treating physician and the consultation and patient-care style of the patient's other diabetes care providers are linked with the standardized prognosis determined from the medical-physiological data, psychological data and the individualized prognosis per patient based on his behavior style (empirical-electronic categorization based on the DISC model or other models). As a result, the individualized, patient-specific prognosis of the course of his condition becomes an individualized, therapeutic recommendation model for controlling the individual patient (with his empirically ascertained and predicted patterns of behavior). This individualized control, which requires minimal effort (since it is ascertained electronically and in an individualized manner, and is linked with a predictive rule), results in a precise, individualized prediction of the patient's behavior, and of the opportunities and risks he will face in his diabetes self-management (compliance).

In addition, information about an individual's self-control (per the DISC/IDEA Style) and a) a general training package (Personal Interaction Training=PIT) for the diabetes care provider, and b) a patient-specific recommendation for behavior are generated electronically for the treating physician and/or other diabetes care providers (nutritionists, psychologists, other medical personnel who are providing medical care that may affect organs in a diabetes-related manner, etc.). The resultant, significant improvement in a patient's behavior in diabetes therapy (compliance), and the resultant patient-specific, individualized support by this patient's diabetes care providers (Individualized Diabetes Therapy) serve to significantly improve the success of diabetes therapy, to protect life (improving the quality of life and life expectancy), and, ultimately, to help reduce the costs of health care, which benefits society as a whole.

In a further advantageous embodiment of the present example embodiment—as another example, but not limited hereto—the patient's behavior style (determined based on the DISC/IDEA categorization system) is related to his “social balance” style, his accuracy in following therapeutic instructions (treatment and adherence), and the success of his therapy (course of glucose values over time: glucose training). The Patient Style Prediction (PSP) indicator is generated. In all, the three parameters of the physician's behavior style of patient management, the physician's social balance, and the physician's compliance management are used to predict the success of therapy based on the Therapy Style Prediction (TSP) indicator based upon the self-assessment data of physician and patient as ONE example of the present technology application. The physician-patient interaction that may be derived from these indicators is analyzed based on the IPS data and using the electronic prognosis that was obtained. It is then incorporated in a DISC/IDEA optimization catalog—which was processed electronically—with four main types (DISC=Driver-Introspective-Supportive-Cooperative/IDEA=Introspective-Driv-er-Expressive-Amiable) and 4×4=16 subtypes (DISC/IDEA test, evaluation scheme) for the patient and for the physician.

PIT for the physician (depending on the type) and for his patient (depending on the type) in the form of computer-aided instruction is thereby made possible.

The three data records named above, i.e., D1=scale of proactive dynamics/dominance (PDD), D2=scale of emotional and communicative openness (EKO), D3=compliance scale (TZ), and the additional scales D4=social balance and flexibility scale (SBE) (D1, D2, D3 and D4 as the prediction group), including a lying scale (D5) for realistically correcting the patient's claims about his eating habits (D6=bolus optimization) are continually refined (D7=prognostic values) and reconciled with the actual data (D8) (validation values: D9).

In a further advantageous embodiment of the present example embodiment, the neural estimators are divided in several modules estimating the several linear independent psychological items resulting from the questionnaire. By this way, a more complex estimation of crossing items between several psychological basis items can be ensured and the personal vector carries/conveys more structured information.

In a further advantageous embodiment of the present example embodiment, the results of the different neural estimators are estimated by a hierarchically higher neuronal neural based “integrator” to an overall estimator of the patient's status.

In a further very advantageous embodiment of the present example embodiment (ADVICE), the sum of all individual patient diagnoses based on a physician or a team of physicians is designed as a learning system or an electronically connected Diabetes Management Network, which—connected via computers, PC's, the internet, and eventually, cellular phones—provides the physician with a computer-aided and wirelessly-supported, therapeutic learning curve that is optimized continually, thereby guaranteeing an asymptotic approximation of optimal diabetes therapy.

Also, a related optimization of the patient's behavior (SMART) with accuracy of the data, (personal compliance) may be realized in one of the above advantageous embodiments of the present example embodiment.

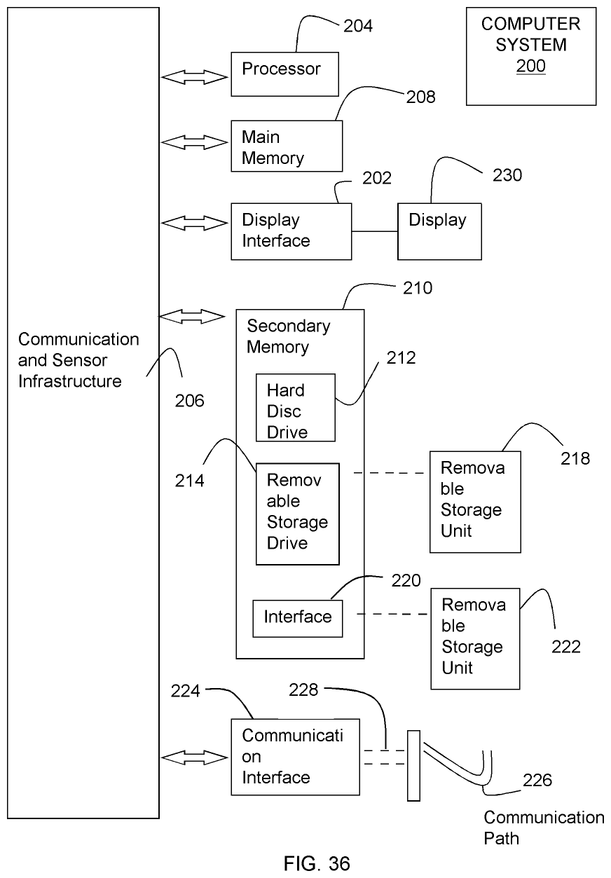

The following are exemplary systems. The method of the example embodiment may be implemented using hardware, software or a combination thereof and may be implemented in one or more computer systems or other processing systems, such as personal digital assistants (PDA's), in blood glucose self-monitoring devices (SMBG memory meters), or systems which administer insulin to a patient; if adequate, memory and processing capabilities are available. In an example embodiment, the example embodiment was implemented in software running on a general purpose computer 200 as illustrated in FIG. 36.

Computer system 200 includes one or more processors, such as processor 204. Processor 204 is connected to communication infrastructure 206 (e.g., a communications bus, cross-over bar, or network). Computer system 200 may include a display interface 202 that forwards graphics, text, and other data from the communication infrastructure 206 (or from a frame buffer not shown) for display on the display unit 230.

Computer system 200 also includes a main memory 208, preferable random access memory (RAM), and may also include a secondary memory 210. The secondary memory 210 may include, for example, a hard disk drive 212 and/or a removable storage drive 214, representing an optical disc drive, a floppy drive, a magnetic tape drive, a flash memory, etc. The removable storage drive 214 reads from and/or writes to a removable storage unit 218, representing an optical disc drive, a floppy drive, a magnetic tape drive, a flash memory, etc. which is read by and written by removable storage drive 214. As will be appreciated, the removable storage unit 218 includes a computer usable storage medium having stored therein computer software and/or data.

In alternative embodiments, secondary memory 210 may include other means for allowing computer programs or other instructions to be located into computer system 200. Such means include, for example, a removable storage unit 222 and an interface 220. Examples of such removable storage units/interfaces include a program cartridge interface (such as that found in video game devices), a removable memory chip (such as a ROM, PROM, EPROM or EEPROM) and associated socket, and other removable storage units 222 and interfaces 220 which allow software and data to be transferred from the removable storage unit 222 to computer system 200.

Computer system 200 may also include a communications interface 224. Communication interface 224 allows software and data to be transferred between computer system 200 and external devices. Examples of communications interface 224 may include a modem, a network interface (such as an Ethernet card), a PCMCIA slot and card, etc. Software and data transferred via communications interface 224 are in the form of signals 228, which may be electronic, electromagnetic such as optical, or other signals capable of being received by communications interface 224. Signals 228 are provided to communications interface 224 via a communications path (i.e., channel) 226. Channel 226 carries signals 228 and may be implemented using wire or cable, fiber optics, a phone line, a cellular phone link, an RF link, an infrared link, and other communications channels.

In this document, the terms “Computer Program Medium” and “Computer Usable Medium” are used to generally refer to media such as removable storage drive 214, a hard disk installed in a hard disk drive 221, and signals 228. These computer program products are means for providing software to a computer system 200. The example embodiment includes such computer program products.

Computer programs (also called computer control logic), structures of neural nets and DISC/IDEA evaluation schemes are stored in main memory 208 and/or secondary memory 210. Computer programs and structures of neural nets may also be received via communications interface 224. Such computer programs, when executed, or such neural net structures or DISC/IDEA evaluation schemes, when used, enable computer system 200 to perform the features of the present example embodiment, as discussed herein. In particular, the computer programs, when executed, enable processor 204 to perform the functions of the present example embodiment. Accordingly, such computer programs represent controllers of computer system 200.

In an embodiment where the example embodiment is implemented using software, the software may be stored in a computer program product and loaded into computer system 200 using removable storage drive 214, hard drive 212 or communications interface 224. The control logic (software), when executed by the processor 204, causes the processor 204 to perform functions to the example embodiment as described herein

In another embodiment, the example embodiment is implemented primarily in hardware using for example, hardware components such as Application Specific Integrated Circuits (ASICs). Implementation of the hardware state machine to perform the functions described herein will be apparent to persons skilled in the relevant art(s).

In yet another embodiment, the example embodiment is implemented using a combination of both hardware and software.

In an example software embodiment of the example embodiment, the methods described above were implemented in SPSS control language, but could be implemented in other programs such as, but not limited to, C++ programming language or other programs available to those skilled in the art.

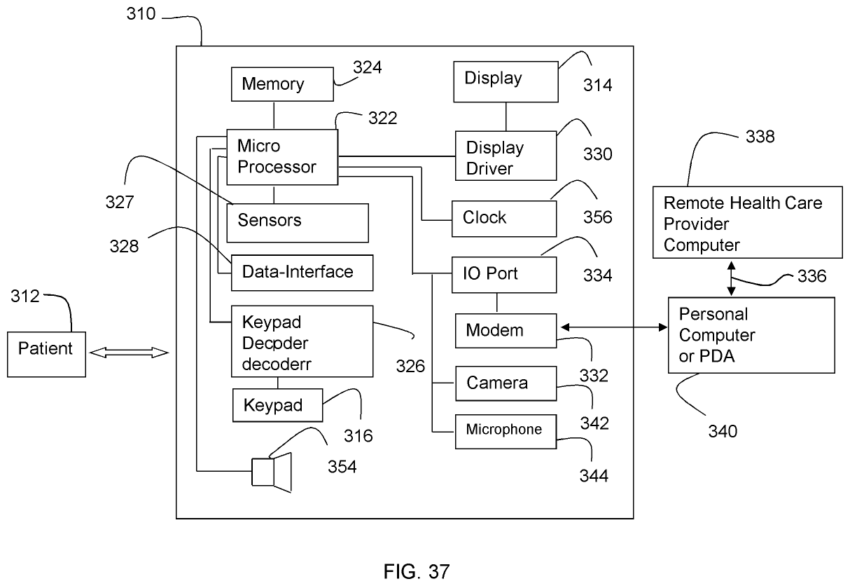

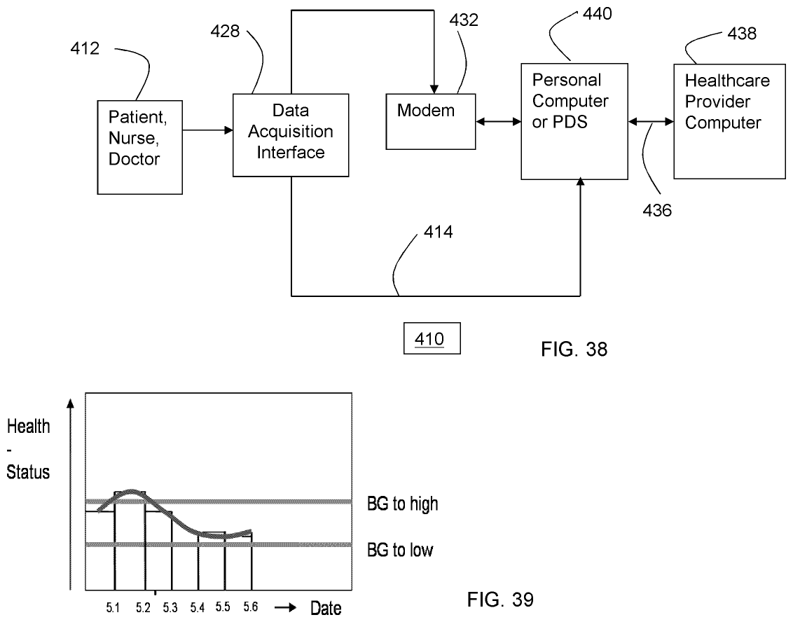

FIGS. 37-38 show block diagrammatic representations of alternative embodiments of the example embodiment. Referring to FIG. 38, there is shown a block diagrammatic representation of the system 410 which essentially comprises the data acquisition device 428 used by a patient or a nurse or a doctor 412 for recording, inter alia, insulin dosage readings and/or measured blood glucose (“BG”), and/or ingested carbohydrate, divided in the three classes fast, medium and slow carbohydrate, and/or psychological parameters of a self-control test. Data obtained by the data acquisition device 428 are preferably transferred through appropriate communication links 414 or data modem 432 to a processing station or chip, such as a personal computer 440, PDA, or cellular telephone, or via an appropriated internet portal. For instance, data stored may be stored within the data acquisition device 428 and may be directly downloaded into the personal computer 440 through an appropriate interface cable and then transmitted via the internet to a processing location. An example is the “ONE TOUCH” monitoring system or meter by LifeScan, Inc., which includes an interface cable to download the data to a personal computer.

Further yet, the data acquisition device 428 or and involved acquisition mechanism may include indwelling catheters and subcutaneous tissue fluid sampling or other devices to intervene in patient's physiology or measure physiological parameters.

The computer PDA 440 includes the software and hardware necessary to process, analyze and interpret the self-recorded diabetes patient data in accordance with predefined flow sequences (as describe above in detail) and generate an appropriate data interpretation output. Preferably, the results of the data by the computer 440 are displayed in the form of a paper report generated through a printer associated with the personal computer 440. Alternatively, the results of the data interpretation procedure may be directly displayed on a video display unit associated with computer 440.

FIG. 37 shows a block diagrammatic representation of an alternative embodiment having a diabetes management system that is a patient-operated apparatus 310 having a housing preferably sufficiently compact to enable apparatus 310 to be hand-held and carried by a patient. A strip guide for receiving a blood glucose test strip (not shown) is located on a surface of housing 316. The test strip is for receiving a blood sample from patient 312. Alternatively, a data-interface 328 or a sensor-interface 327 can be used, depending on which equipment of the data acquisition is in use. The apparatus includes a microprocessor 322 and a memory 324 connected to microprocessor 322. Microprocessor 322 is designed to execute a computer program stored in memory 324 to perform the various calculations and control functions as discussed in great details above. A keypad 316 is connected to microprocessor 322 through a standard keypad decoder 326. Display 314 is connected to microprocessor 322 through a display driver 330. Microprocessor 322 communicates with di splay driver 330 via an interface, and display driver 330 updates and refreshes display 314 and the control of microprocessor 322. Speaker 354 operates under the control of microprocessor 322 to emit audible tones alerting the patient to possible future hypoglycemia or hyperglycemia or gives word of advice what to do next to prevent hypoglycemia or hyperglycemia. Clock 356 supplies the current date and time to microprocessor 322.

Memory 324 also stores the locked data of the patient 312, the insulin dose values, the insulin types, and the parameter values used by microprocessor 822 and the neural net structures to calculate future blood glucose values, supplemental insulin doses, and carbohydrate supplements. Each blood glucose value and insulin dose value is stored in memory 324 with a corresponding date and time. Also, the neural net structures and the potential adapted neural net structures, the old and potential modified data of the self-test and the optimization parameters are stored in memory 324 with a corresponding date and time. Memory 324 is preferably a non-volatile memory, such as an electrically erasable read only memory (EEPROM). This kind of memory is preferably also in use for the alternative embodiments discussed before.

Apparatus 310 further includes an input/output port 334, preferable a serial port, which is connected to microprocessor 322. Port 334 is connected to a modem 332 by an interface, preferably a standard R232 interface. Modem 332 is for establishing a communication link between apparatus 310 and a personal computer 340 or a healthcare provider computer 338 through a communication network 336. Specific techniques for connecting electronic devices through connection cords are well known in the art. Another alternative example is “Bluetooth” technology communication. A still or video camera 342, and a microphone 344 can be in operable communication with the microprocessor 322 for utilization in direct audio and/or video chat communication between the patient and the HCP.

Accordingly, the embodiments described herein are capable of being implemented over data communication networks such as the internet, making evaluations, estimations, and information accessible to any processor or computer at any remote location, as depicted in FIGS. 36-38 and/or U.S. Pat. No. 5,851,186 to Wood, which is hereby incorporated by reference herein. Alternatively, patients located at remote locations, may have their data transmitted to a central healthcare provider or residence, or a different remote location.

In particular, the present example embodiment provides a method for the predictive control of a biological organism using self-adaptive model structures, wherein the state quantities that describe an organism, i.e., the physiological and medical data that are ultimately measured and four psychological behavior style components—which are categorized based on a specified matrix structure of the organism according to the criteria (D-I-S-C/I-D-E-A) and are summarized in 16 subcategories—are ascertained and supplied to a suitable device that categorizes them so that, based on this categorization, handling instructions for the physician and patient are derived, which results in an improved “prediction of the bolus values and insulin requirements of patients with diabetes”.

In particular, the present example embodiment provides a method for the predictive control of a biological organism using self-adaptive model structures, wherein, based on the results of the handling instructions described in the previous paragraph, requirements for improved therapy provided by diabetes physicians to patients with diabetes may be derived using a suitable device by incorporating the psychological data and typology definition (DISC/IDEA) and, as a further measure, incorporating the patient's compliance over time by determining a patient's requirements for future therapy based on therapy already completed and the state quantities that were measured and that describe the organism using an adaptable expert system or an additional, model-based system of categorization.

In particular, the present example embodiment provides a method for the predictive control of a biological organism using self-adaptive model structures, wherein the following method steps are carried out using a sensor device that repeatedly ascertains the DISC/IDEA data and supplies them to an electronic device, which combines them to form a vector using a program-controlled, digital device:

(1) Supply the sensor data vector to a computer-aided, statistical estimator structure, i.e., a neural network, the program of which is designed such that:
(1.1) its elements (neurons) are ordered such that they form a topologically closed, two-dimensional surface (a torus) on a regular or irregular grid;
(1.2) all elements of the estimator estimate the structure of the one sensor data vector (SV) with a value between zero and one, these estimates being carried out—while retaining similarities between various sensor data vectors—such that, using a mathematical formula stored in the processor, these similarities are reflected in similar estimates and such that they are topologically adjacent, while retaining topological interrelationships;
(1.3) in a selectable operating state, “conditioning”, the behavior of all estimators and/or the parameters that characterize them are adjusted automatically via the input of unknown (not yet presented) sensor data vectors—provided they differ to a certain extent, to be defined a priori, from all sensor data vectors presented up to that point—, the adjustment representing a refinement of the ability of the estimator to categorize and, therefore, representing a refinement of the model structure of the system to be controlled, without increasing the number of estimators; and
(1.4) in a selectable operating state, “classification”, any sensor data vectors of the sensor data vectors presented previously and stored in the structure of the estimator are assigned to the measure described above, based on similarity, so that model structures of the system to be controlled are represented by the totality of all estimator values.

(2) The totality of all values of the estimator are supplied to a second computer-aided, statistical estimator structure, which is designed such that it calculates adequate regulating parameters for the system to be regulated from the totality of all values of the first estimator, in which case:
(2.1) its elements (neurons) are ordered such that they correspond to a neural feed-forward structure,
(2.2) in a selectable operating state, “conditioning”, the structure of the second estimator may be designed such that a defined totality of all values of the first estimator corresponds to a certain control instruction for the system to be controlled,
(2.3) in a selectable operating state, “classification”, any totalities of all values of the first estimator are assigned to certain control instructions.

In particular, the present example embodiment provides a system using a prediction system of physiological data for establishing a target function for an optimization algorithm, which is implemented in a computer system. With this optimization algorithm, optimal decisions for individual use or medical advice can be computed.

Preferably, in the above method or system, a target function, Equation 6

$$t = \sqrt{\frac{\int {( G - {G}_{t} )}^{2}}{T}} \qquad Equation 6$$

is used, where G is the ideal glucose value, Gt is the glucose value at time t and T is the measurement period.

Preferably, in the above method or system, cross validation methods are used for reducing necessary patient records.

Preferably, in the above method or system, the prediction correctness (failure measure) of the predictor is appraised with a function as, Equation 7:

$$t = \sqrt{\frac{\int {( G - {G}_{t} )}^{2}}{T}} \qquad Equation 7$$

where G is the ideal glucose value, Gt is the glucose value at time t and T is the measurement period.

Preferably, in the above method or system, missing values for patient records in a cross validation or learning process are substituted by values delivered by a predictor as described (failure measure with predictor values).

Preferably, in the above method or system, a patient's record is classified into risk classes, which are comprised of parameters describing the glucose value and the controllability of the patient's behavior.

Preferably, in the above method or system, the prediction correctness for the glucose value for a patient as a parameter describing the risk classes is used.

Most preferably, in the above method or system, a patient is given a feedback with described risk classes.

Preferably, in the above method or system, input data are delivered by a continuous sensor system, i.e. a connection with sensor data is provided.

Preferably, in the above method or system, cross validation and scoring methods for selecting of optimal parameters are used.

Preferably, in the above method or system, cross validation for categorizing patient cohorts by a physiological description is used, i.e. cross validation for assessing DISC/IDEA clusters is used.

Preferably, in the above method or system, based on a detected surplus of insulin at any given time, a prediction is made which indicates that a certain amount of additional carbohydrates may be ingested.

Preferably, in the above method or system, a patient DISC/IDEA class is used as an input value for the prediction system, i.e. DISC/IDEA categories serve as an input for the prediction.

Preferably, in the above method or system, patient records for a learning procedure are separated in DISC/IDEA categories, i.e. a stratification with DISC/IDEA categories is carried out.

In summary, the example embodiment proposes a data analysis computerized (or non-computerized) method and system and a patient specific estimations structure method and system for the simultaneous evaluation of significant components of glycemic control in individuals with diabetes: BG and the risk of hyperglycemia and hypoglycemia combined with a predictor for the early and further future BG-courses, the personal medical records and advice for an optimal therapy. The method provides, among other things, four sets of output.

The potential implementations of the method, system, and computer program product of the example embodiment are that it provides the following advantages, but are not limited thereto. First, the example embodiment enhances existing home BG monitoring devices by producing and displaying: 1) estimated categories for BG, 2) estimated probability for SH in the subsequent six month, 3) estimated short-term risk of hyperglycemia and hypoglycemia (i.e. for the next 24 hours), 4) estimated doses of insulin requirements to fix the BG in a favored interval. The latter may include warnings, such as an alarm, that indicates imminent hyperglycemia and hypoglycemia episodes. These four components can also be integrated to provide continuous information about the glycemic control of individuals with diabetes, and to enhance the monitoring of their risk of hyperglycemia and hypoglycemia.

As an additional advantage, the intention enhances existing software or hardware that retrieves measured BG data.

Moreover, another advantage, the example embodiment evaluates the effectiveness of various treatments for diabetes and changes of the medical report caused by the lifestyle of the patient.

Further still, as patients with diabetes face a life-long optimization problem of maintaining strict glycemic control without increasing their risk of hyperglycemia and hypoglycemia, the present example embodiment alleviates this related problem by use of its individual patient model, which “learns” over time the special psychological and physiological behavior of the patient and adapts 1) the succession of the necessary insulin doses to fix an optimal BC, 2) the advice what to do next to prevent hyperglycemia and hypoglycemia, 3) the standards for optimizing the therapy of the patient.

Another advantage, the example embodiment evaluates the effectiveness of new insulin or insulin delivery devices. Any manufacturer or researcher of insulin or insulin delivery devices can utilize the embodiments of the example embodiment to test the relative success of proposed or tested insulin types or device delivery designs.

Finally, another advantage, the example embodiment evaluates the effectiveness of drugs that are adjunct to insulin therapy.

### EXAMPLE

This example consists of seven algorithms for simultaneous evaluation, from routine collected physiological data blood glucose, ingested carbohydrate—divided in the three classes fast, medium and slow carbohydrate—and psychological parameters of the self-control test DISC/IDEA to prevent hyperglycemia and hypoglycemia, to calculate the BG value in the early next future (24 hours), to give indications about how to optimized the daily behavior of the patient in the context of an evidence-based medicine, to optimize the therapy, and to verify the daily behavior of selected groups to optimize the DISC/IDEA categorization. This method pertains directly to enhancement of existing BG monitoring devices, an existing DISC/IDEA categorization, the mathematical procedure of cross-correlation, a standard to divide the three classes: fast, medium and slow carbohydrates and the knowledge about periods of an increasing risk for hyperglycemia and hypoglycemia. The basis of this method is an individual computer-based model of a diabetes patient. The data analysis method has seven components (algorithms) all advecting in the patient's computer model.

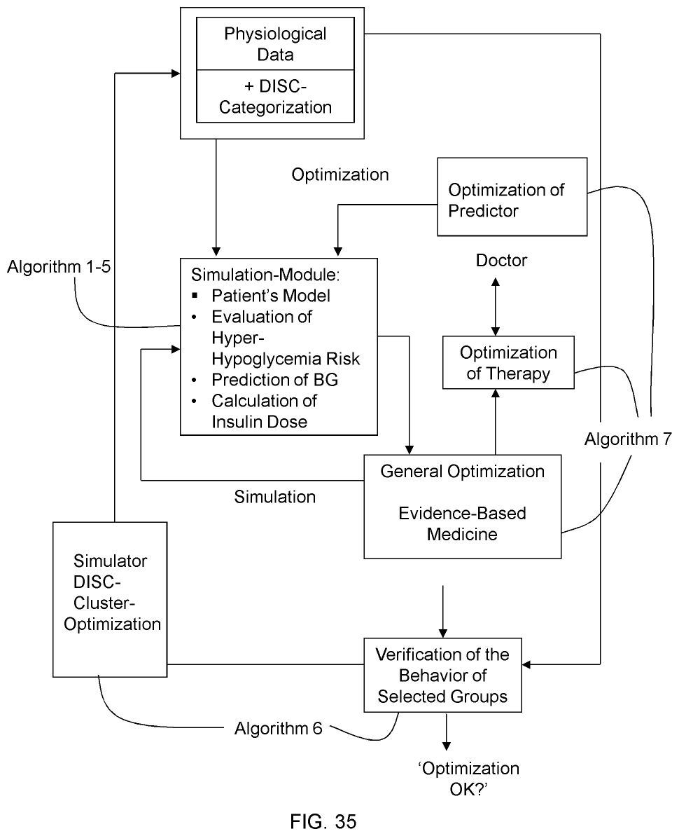

The following Algorithms (see FIG. 35) can be utilized in or by the present technology:

Algorithm 1: Creation of an individual patient's model

Algorithm 2: Calculation/prediction of the BG in given time interval

Algorithm 3: Evaluation of long-term risk for severe hyperglycemia and Hypoglycemia

Algorithm 4: Evaluation of short-term (within 24-48 hours) risk of hyperglycemia and hypoglycemia

Algorithm 5: Calculation of necessary insulin doses to fix the patient's BG in a favorite interval

Algorithm 6: Verification of behavior of the selected groups to optimize the DISC/IDEA categorization

Algorithm 7: Verification of therapy and predictor to optimize both

Algorithms 1, 2, 3 and 5 provide uninterrupted monitoring and information about the overall glycemic control of an individual with type 1 or type 2 diabetes mellitus (T1DM, T2DM), covering both the high and low ends of the BG scale.

Algorithm 4 is supposed to be activated when Algorithm 3 indicates an increasing long-term risk for hyperglycemia or hypoglycemia. Upon activation, Algorithm 4 requires more frequent monitoring (4 times a day) and provides a 24 to 48-hour forecast of the risk for moderate/severe hyperglycemia and hypoglycemia.

Data sets can be utilized by or in the present technology.

In order to ensure that the results of our optimization can be generalized to population level, Algorithms 1, 2, 3 and 4 were first optimized using training data sets and tested for accuracy using unrelated test data sets.

For Algorithm 5, standard values for the administration of insulin doses were used that were verified by Algorithms 2, 3 and 4.

For Algorithm 6, three special studies (Annex IS₁, IS₂, IS₃) have been carried out:

(1) IS₁: The Proof of Concept Study with n=131 diabetes patients of type 2 and type 1 in February-March-April 2009 verified that 100% of the patients accepted the resulting Individual Personal Profile with the personal style (English version I-D-E-A, German version A-D-E-L=Analytic (Analytiker)-Driver-Expressive(r)-Amiable/Liebenswerter) as correct and realistic description of their personal style.

(2) IS₂: In a second study with n=1,000 patients, a multiple regression analysis and a factor analytic study led to the categorization of the patients and was verified by an analysis of the Secondary Factors which indicate the 3 Categories of patients (E & E=Enabling and Empowerment, C & C=Communication and Control, A & C=Adaptation (of Lifestyle) and Coping). The 9 Success factors of Diabetes Management have been identified (which was later on a 10 Factor Scheme, since the two component factor 3 of the IS₂-Study Motivation and Knowledge of Diabetes Management was later on differentiated in the German Study IS₃ into two separate factors: F3=Motivation (and Energy) and F4=Knowledge of Self-Care.

(3) IS₃: The results of the n=1,000 US Study (IS₂) were verified and reinforced by the third study IS₃ (Study 3 of the present technology), carried out in November (Pre Studies) and December (Final Contract Study) 2010:
relevance of style for behavior, adaptation and communication (multiple correlation R=0.45);
verification of 9 (10) success factors;
classification of patients in three categories (E & E, C & C, A & C).

For training Data Set 1: 15 data sets representing standard patients which eat fast carbohydrate and inject a large dose of insulin (sub-cutaneously) and describing the BG-behavior of the patient's metabolism over a 24 h period.

For training Data Set 1: 15 data sets representing standard patients which eat medium carbohydrate and inject a large dose of insulin (sub-cutaneously) and describing the BG-behavior of the patient's metabolism over a 24 h period.

For training Data Set 1: 15 data sets representing standard patients which eat slow carbohydrate and inject a large dose of insulin (sub-cutaneously) and describing the BG-behavior of the patient's metabolism over a 24 h period.

For training Data Set 1: 15 data sets representing standard patients which eat fast carbohydrate and inject a medium dose of insulin (sub-cutaneously) and describing the BG-behavior of the patient's metabolism over a 24 h period.

For training Data Set 1: 15 data sets representing standard patients which eat medium carbohydrate and inject a medium dose of insulin (sub-cutaneously) and describing the BG-behavior of the patient's metabolism over a 24 h period.

For training Data Set 1: 15 data sets representing standard patients which eat slow carbohydrate and inject a medium dose of insulin (sub-cutaneously) and describing the BG-behavior of the patient's metabolism over a 24 h period.

For training Data Set 1: 15 data sets representing standard patients which eat fast carbohydrate and inject a small dose of insulin (sub-cutaneously) and describing the BG-behavior of the patient's metabolism over a 24 h period.

For training Data Set 1: 15 data sets representing standard patients which eat medium carbohydrate and inject a small dose of insulin (sub-cutaneously) and describing the BG-behavior of the patient's metabolism over a 24 h period.

For training Data Set 1: 15 data sets representing standard patients which eat slow carbohydrate and inject a small dose of insulin (sub-cutaneously) and describing the BG-behavior of the patient's metabolism over a 24 h period.

Algorithm 1 can be utilized for the creation of an individual patient's model

Example No. 1 provides for, without being limited thereto, a mathematical model, representing a patient, with varying eating and injecting behavior.

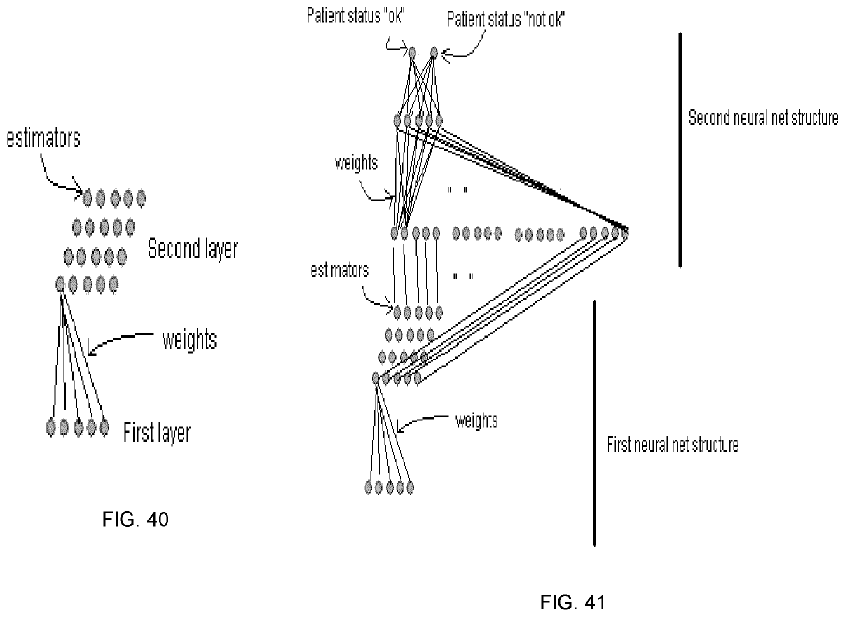

Algorithm 1 includes a neural net following the self-organizing-principle for sorting diverse personal status vectors (PSVs) to representing activity patterns on a closed grid. An exemplary structure of these kinds of nets is shown in FIG. 40 and an exemplary closed grid in FIG. 42.

In Algorithm 1, the first layer is constituted of 12 estimators, i.e., the 10 Core Instruments [see communication system of the present technology) with 10 Core Instruments (especially Core No. 1=Individual Diabetes Status and Core No. 2=IPP=“Individual Personal Profile”, Core No. 11=“Individual Stress Test” (IST), and Core No. 12=“Individual Measurement Behavior” (IMB)].

These estimators are comprised to 10 Success factors and 3 Patient Categories. They are representing some (for example, but not limited to) 267 items of the communication system of the present technology=ICS.

The second layer, which represents the neuro-mental patient's model itself, is constituted of 12 times 12 neurons/estimators. To create the patient's model, we proceed as follows.

The initial learning rate ε^{t} was set to 0.9, the initial adoption function δ^{t} to 0.98. For the neighborhood function h^{t}ₛᵢ the Gaussian function was selected. The initial radius of the influence of h^{t}ₛᵢ; was the grid radius.

A total of 10 data sets of every patient's type were presented in random order to the neural net structure. The weights were adapted in 1,100 conditioning operation state steps.

Detailed estimations of the validity of the neural net were made using the test data sets only.

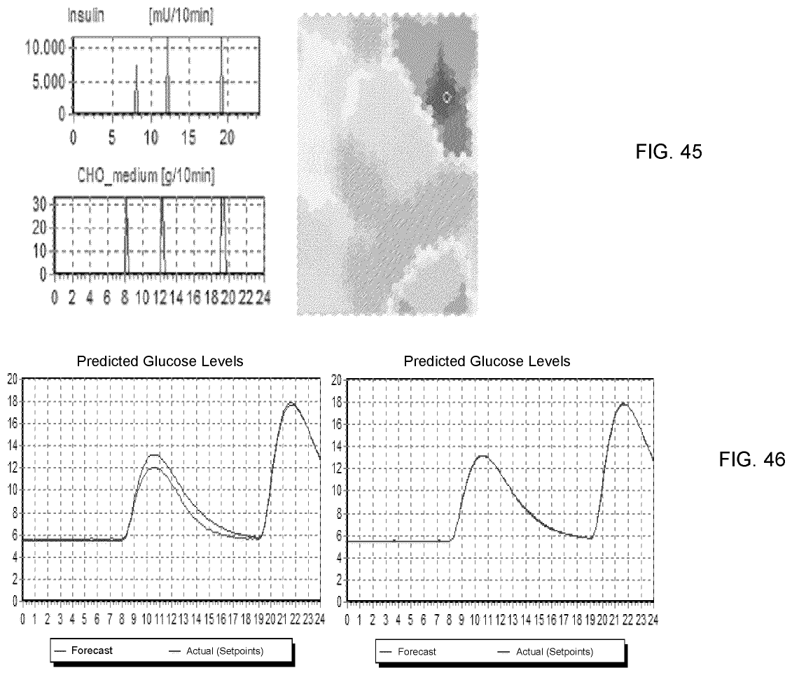

This separation of training and test data sets allows us to claim that the estimated preciseness of Algorithm 1 be generalized to any other data of subjects following the physical and psychical standard of the used patient category. Moreover, when we present test PSV-sets which slightly differ from the learning PSV-sets we can observe, that the activity pattern also differed only slightly, with a significance of 0.98 calculated with a standard t-test. An exemplary visualization of a PSV and the corresponding activity pattern of the neural net are shown in FIG. 45.

Algorithm 2 can be utilized for the calculation/prediction of the BG in given time interval.

Example No. 1 provides for, without being limited thereto, an expansion of Algorithm 2 to include estimating individual probabilities for the blood glucose (BG) in a defined time in the future, which in this example, without loss of generality, was limited to a maximum to 24 hours.

Algorithm 2 includes a neural net resp. a hierarchical neural net battery, whereby all of them follow the self-organizing-principle for sorting diverse personal status vectors (PSV's) to representing activity patterns on a closed grid and a neural net integrating the estimations of the SOM's, to an overall patient behavior model, followed by a neural net, following the backpropagation algorithm to decode the activity pattern of the lower neural net hierarchy. In that way, a predicted BG-value is calculated for a defined time window.

Example (FIG. 41): An exemplary structure of these kinds of nets is shown in FIG. 41. In Algorithm 2, the first layer is constituted of 2,320 estimators.

The “number” and “power” of these estimators is extracted and “condensed” from the complete patient information of the BPPS model, for instance (but not limited hereto) 232 items of 10 Core Instruments of the communication system of the present technology (but not limited to this exemplary model) with 10 Success factors for Diabetes Management (see Study IS₂, USA, n=1,000; IS₃, Germany, n=2,358).

These 2,320 neurons/estimators (defined by the BPPS model information of 232 items, 10 Core Instruments, 10 Success factors and 3 Secondary Factors or Patient Categories) represent the features of a patient's neuronal category/status at a given time T.

If a time scale, for example 10 minutes, is chosen and a prediction level of 4 his selected, 24 PSV's, representing 4 hours of history.

The second layer, which represents the neuro-mental patient's model itself, is constituted of 12 times 12 neurons/estimators. To create the patient's model, we proceed as follows.

The initial learning rate ε^{t} was set to 0.9, the initial adoption function h^{t}ₛᵢ, to 0.98. For the neighborhood function h^{t}ₛᵢ the Gaussian function was selected. The initial radius of the influence of h^{t}ₛᵢ was the grid radius.

A total of 10 data sets of every patient's history were presented in random order to the neural net structure. The weights were adapted in 1,100 conditioning operation state steps.

Detailed estimations of the validity of the neural net were made using the test data sets only.

This separation of training and test data sets allows us to claim that the estimated preciseness of Algorithm 2 be generalized to any other data of subjects following the physical and psychical standard of the used patient category. Moreover, when we present test PSV-sets which slightly differ from the learning PSV-sets, we can observe that the activity pattern also differed only slightly, with a significance of 0.96 calculated with a standard t-test.

To calculate the predictive regime of the BG we proceed further on as follows:

The activity pattern of the neural net hierarchy 1 was presented to the integration SOM of hierarchy 2 and its resulting activity pattern to the input layer of the backprogation neural net (neural net 2), whereby the input layer of the backpropagation neural network structure has 144 neurons/estimators, the hidden layer 30, while the number of estimators in the output layer can be chosen freely and can be as many estimators as required. The initial learning rate s was set to 0.75.

A total of 10 data sets of every patient's history were presented in random order to the neural net 2. The weights were adapted in 2,000 conditioning operation state steps until the desired BG-value regime was decoded out of the activity pattern of the neural net 1.

Detailed estimations of the validity of the neural net were made using the desired BG-values data sets and comparing them with the BG data regimes the net-structure calculates.

Also, detailed estimations of the validity of the neural net were made using the test data sets only.

The separation of training and test data sets allows us to claim that the estimated preciseness of Algorithm 1 be generalized to any other data of subjects following the physical and psychical standard of the used patient category. Moreover, when we present test PSV-sets which slightly differ from the learning PSV-sets, we can observe that the activity pattern also differed only slightly, with a significance of 0.97 calculated with a standard t-test. An exemplary visualization of a not yet well predicted BG-regime (left-hand side) and a well predicted BG-regime (right-hand side) are shown in FIG. 46.

Algorithm 3 can be utilized for the evaluation of long-term prediction of the patient's status.

Example No. 1 provides for, without being limited thereto, an expansion of Algorithm 2 to include estimating individual probabilities for biochemically significant hypoglycemia (BSH, defined as BG reading<=39 mg/dl) or biochemically moderate hypoglycemia (BMH, defines as 39 mg/dl<BG reading<=55 mg/dl) or the development of the patient's status in the future.

Algorithm 3 is a classification algorithm. That is, based on SMBG data for a subject, it classifies the subject in a certain risk category for future BSH or MSH. In order to approximate as closely as possible future real applications of Algorithm 3, we proceed as follows:

From the individual patient's model and from the calculation/prediction of the BG in a given time interval and from the DISC/IDEA-categorization, an enlarged PSV data set is created.

As described above, a neural predictor is created to calculate the probability for biochemically significant hypoglycemia or biochemically moderate hypoglycemia, and advice is given when these events occur to a significant degree. Also, it is created to calculate the probability for patient's psychological behavior in time.

Detailed estimations of the validity of the neural net were made using the desired BG-values data sets and comparing them with the BG data regimes the net-structure calculates.

Also, detailed estimations of the validity of the neural net were made using the test data sets only.

The separation of training and test data sets allows us to claim that the estimated preciseness of Algorithm 1 be generalized to any other data of subjects following the physical and psychical standard of the used patient category. Moreover, when we present test PSV-sets which slightly differ from the learning PSV-sets, we can observe that the activity pattern also differed only slightly, with a significance of 0.93 calculated with a standard t-test.

Algorithm 4 can be utilized for the evaluation of short-term risk for severe hyperglycemia and hypoglycemia.

Example No. 1 provides for, without being limited thereto, an expansion of Algorithm 3 to include estimating individual probabilities for biochemically significant hypoglycemia (BSH, defined as BG reading<=39 mg/dl) or biochemically moderate hypoglycemia (BMH, defines as 39 mg/dl<BG reading<=55 mg/dl) and/or patients psychological behavior in a short time.

Algorithm 4 is a classification algorithm following the same principles as the algorithm for the long-term risk, described above. In order to approximate as closely as possible future real applications of Algorithm 4, we proceed as follows:

From the individual patient's model and from the calculation/prediction of the BG in given time interval and from the DISC/IDEA-categorization an enlarged PSV-data set is created.

As described above, a neural predictor is created to calculate the probability for biochemically significant hypoglycemia or biochemically moderate hypoglycemia, and advice is given when these events occur to a significant degree in the early next (immediate) future.

Detailed estimations of the validity of the neural net were made using the desired BG-values data sets and comparing them with the BG data regimes the net-structure calculates.

Also, detailed estimations of the validity of the neural net were made using the test data sets only.

The separation of training and test data sets allows us to claim that the estimated preciseness of Algorithm 1 be generalized to any other data of subjects following the physical and psychical standard of the used patient category. Moreover, when we present test PSV-sets which slightly differ from the learning PSV-sets, we can observe that the activity pattern also differed only slightly, with a significance of 0.945 calculated with a standard t-test.

Algorithm 5 can be utilized for the calculation of necessary insulin doses to fix the patient's BG in a favorite interval.

Example No. 1 provides for, without being limited thereto, an expansion of Algorithm 4 to include calculating the individual dose for an insulin injection to fix the patient's BG in a favorite interval.

Algorithm 5 is a Calculating Module. In Order to Approximate as Closely as Possible Future Real Applications of Algorithm 5, we Proceed as Follows:

From the individual patient's model and from the calculation/prediction of the BG in given time interval and from the DISC/IDEA-categorization and from the patient's physical data like weight, drug abuses, sports activities, sex etc., an insulin dose is calculated to fix the BG in the given time window in a favorite interval.

This dose is given as input component to the individual patient's model, which calculates by means of the representation of the patient's physiology the BG values in the early next future, preferably in the next 3 hours, by the help of the prediction algorithm described above.

Algorithm 6 can be utilized for the verification of behavior of the selected groups to optimize the DISC/IDEA-Categorization.

Example No. 1 provides for, without being limited thereto, an expansion of Algorithm 2 to include estimating individual probabilities for an optimal patient's model by changing the DISC/IDEA-parameters.

Algorithm 6 is a calculating module based on statistical formula methods. In order to approximate as closely as possible future real applications of Algorithm 6, we proceed as follows:

From the individual patient's model and from the calculation/prediction of the BG in a given time interval and from the DISC/IDEA-categorization and from the patient's physical data like weight, drug abuses, sports activities, sex etc., an individual adaptation of the DISC/IDEA-categories are calculated.

Algorithm 7 can be utilized for the verification of therapy and predictor to optimize both.

Example No. 1 provides for, without being limited thereto, an expansion of Algorithm 2 to include estimating individual probabilities for an optimal patient's model by changing the DISC/IDEA-parameters.

Algorithm 7 is a calculating module based on statistical formula methods. In order to approximate as closely as possible future real applications of Algorithm 7, we proceed as follows:

An optimization module with a target function, Equation 8

$$t = \sqrt{\frac{\int {( B G - B {G}_{t} )}^{2}}{T}} \qquad Equation 8$$

whereby BG describes the ideal glucose value, BGₜ the glucose value at time t and T the measurement period, is implemented to calculate the optimal glucose concentration for individual use or medical advice.

Using cross validation methods, it is calculated if the necessary patient records can be reduced.

The prediction correctness of the predictor is appraised by implementing the function, Equation 9:

$$t = \sqrt{\frac{\int {( G - {G}_{t} )}^{2}}{T}} \qquad Equation 9$$

whereby BG describes the ideal glucose value, BGₜ the glucose value at time t and T the measurement period.

Missing values for patient records in a cross validation or learning process are substituted by values delivered by the predictor as described above.

Patient's records are classified into risk classes. These risk classes are comprised of parameters describing the glucose value and the controllability of the patient's behavior.

The prediction correctness for the glucose value for a patient is used as a parameter describing the risk classes.

These risk classes are returned to the patient as a feedback.

The example embodiment may be embodied in other specific forms without departing form the spirit or essential characteristics thereof. The foregoing description of examples, embodiments, etc. is therefore to be considered in all respects illustrative rather than limiting of the example embodiment described herein. Scope of the example embodiment is thus indicated by the appended claims rather than by the forgoing description, and al changes, which come within the meaning and range of equivalency of the claims, are therefore intended to be embraced therein.

The system according to the example embodiment may include an individualized disease management system of the present technology, which operates on four integrated program modules.

1. SCTWEB Module© (Survey Construction Tool Web Module)

The SCTWEB MODULE© contains a database tool in which we can put validated items and descriptors into categories which forms the basis of the survey of the present technology.

The SCTWEB MODULE© is prepared to handle all western languages and Cyrillic languages like Ukrainian and Russian languages.

Once a survey is set-up in the SCTWEB MODULE©, this tool publishes the questionnaire of the present technology on the internet and is approachable for the browsers: Internet Explorer (6 and 7 and higher), Firefox (2 and 3) and Safari (3,0). This accounts for 98% of the browser market.

2. The CSM Module© (Client and Survey Management Module)

The CSM Module contains several databases in which we register the healthcare provider who will participate in the survey of the present technology. This can be done per region, in the USA per state and in Europe per country.

Δt that moment, the healthcare provider is registered into the system using an e-mail with his password sent to him. As a result, the healthcare provider is able to setup his patient database.

From that moment on the healthcare provider can create a survey out of the modules that are set in the SCTWEB Module© and send that survey to the patient.

Δt the moment the patient is matched to the survey that the healthcare provider created the system creates and individualized ID-number that gives a patient entrance into two internet pages. The first page is the survey page and the second page is his/her personal portfolio page.

Every patient receives an individual portfolio page on the internet on which he can see his “concept-report” and can create his/her final report also with comments/remarks her/himself to send to the healthcare provider.

Also, the CSM Module automatically creates a healthcare provider control page on which—after authorization of the patient—the healthcare provider finds the report from the patient.

The present technology system is set up in in such a way that based on real-time scoring the “concept-reports” will be published on the portfolio page of the patient, instantly after filling in the questionnaire, and that there is instant publishing of the report on the healthcare provider control page after authorization and establishment of the final report of the patient.

The system will send out automatically an announcement by e-mail to the healthcare provider that the report of the patient is available.

3. The RG Module© (Report Generator Module)

The RG Module is the survey scoring program. It is designed to build the reporting template for the report lay-out. Reports are placed on management control pages and personal portfolio pages.

4. The PC Module© (Patient Communication Module)

The PC Module is integrated in the CSM module and sets the communication templates for patients, respondents and health care providers.

Based on the content of the CSM module, the PC module notifies and instructs how to approach a survey, sends out reminders, notifies respondents, reminds respondents, notifies when reports are published etc.

5. Safeguards of the Present Technology System

The service centers of the present technology are linked to our data centers.

Data centers can be positioned in Amsterdam in the exemplary (we lease several dedicated servers at Denit (our provider) with a 99.8% performance service level agreement.

The back-up systems for the dedicated servers are positioned in Norway at Denit, in the exemplary. As an extra safeguard, there are two real time extra back-up data centers on our own servers, one in Rotterdam (WVD media) and one in Zwijndrecht (Service Center of the present technology), in the exemplary.

So in case of emergency in Amsterdam, we automatically switch to the back-up systems and then we have a maximum loss of data entry of 1 hour.

All processing centers are logged in to our data centers through the internet.

For the present technology project we place extra high speed/high capacity servers in the data centers which provide capacity for a minimum of 1,000,000 data entries and storage reports a year, to guarantee real time scoring and processing (January 2011).

The system according to the example embodiment may include a Report Generator Module.

The Report Generator Module is an application that works in cooperation with the portfolio system and the “Survey Construction Tool (SCT). The Report Generator Module is used to make reports for survey trajectories and is specialized in making personalized reports for surveys.

In this document, we firstly describe the key concepts and basic flow of the program. Then we go into more detail on how to use the report generator to achieve the Basic Individual Profile (BIP) Reports and Promptsheets.

As mentioned before, the report generator module works together with the Portfolio System and the Survey Construction Tool.

FIG. 47 shows the way the communication is constituted. Data is downloaded to the report generator, reports are generated and those reports are finally uploaded back to the portfolio system to be available online.

A report generator can be included with the present technology. Although some aggregate functions are available, with the report generator a report is always created for a specific patient. Reports generated using the report generator can automatically be uploaded back to the portfolio system so that it is available to both participants (via the personal portfolio page) and doctors of HCP's (through the portfolio status page).

Usually, every survey can be different from the next. Hence, the resulting reports are likely to vary in structure as well. That is why the report generator works with templates/forms, i.e. partially complete documents to be filled in and that can be reused and adapted and hence provide the demanded flexibility.

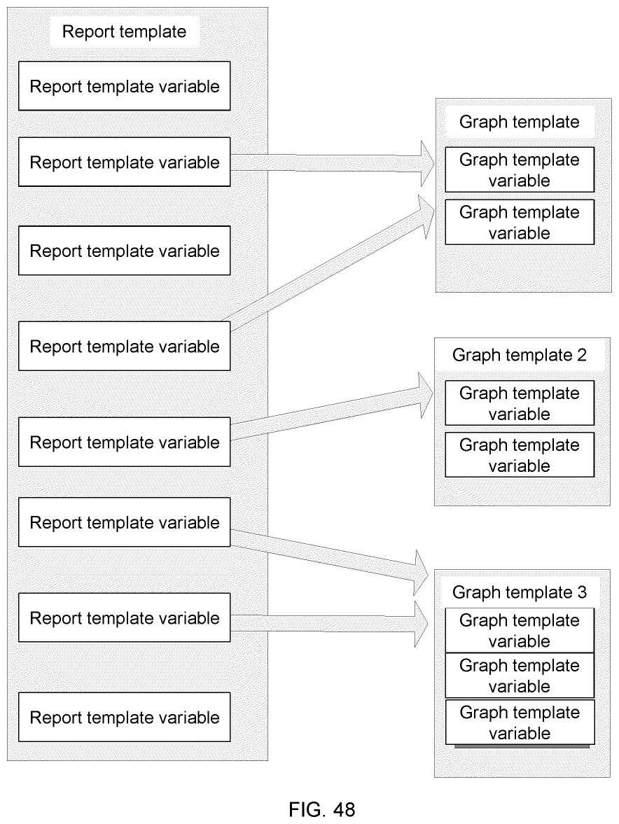

FIG. 48 is a schematic diagram showing how report templates and graph templates are related. On the basis of every report, there is a report template. A report template may be a Microsoft Word XML document and hence may be edited using the widely supported Microsoft Word XML.

A report template contains report template variables. A report template variable is a part of the report that is replaced by actual survey data once a report is generated. There are different types of report template variables. One of them is a reference to a graph template. A graph template shows information (averages, totals) about a specific survey question or a group of survey questions. A graph template is also a Microsoft Word document, but can contain embedded Excel Chart or Microsoft Chart objects. Graph template contains graph template variables. A graph template variable is replaced by a (numerical) value upon report generation.

With the report generator we can create two types of reports: individual patient reports and general reports. Individual reports are reports that are created for specific patients and doctors such as BIP profiles and promptsheets. In those reports, data is used that is linked to a specific patient. The patients and doctors are being entered in the portfolio system under a Client management section. General reports are reports that use all survey data, for instance for a group of patients for one doctor. Those kinds of reports are used to report on the higher organizational level and not for specific patients.

Based on various settings that can be modified using the user interface, the report generator will insert one or more texts in a report.

All texts reside in separate Word XML documents that have particular, pre-set filenames. These documents all reside in a folder that should have a certain, pre-defined structure. Which texts are inserted and where, depends on the place of the variables used in the report template.

As mentioned before, the report generator also works in cooperation with the survey construction tool. Hence, it is possible to display information from the SCT.

Depending on the settings in the user interface and of course the patient's survey data, a text is inserted in the report. All individual texts are stored in separate Word documents that reside in a predefined folder structure. There are a number of report variables, that each inserts (each of which inserts) a different text into the document.

The system and method according to the example embodiment enables clients/patients and HCP/doctors to remove typical communication barriers in existing health care systems. By providing a data communication platform within the system of the example embodiment for communicating relevant physiological (medical, biological) and psychological (personality profile determined using psychometry) information among the individuals involved in and being part of the system. Thus, the clients/patients and HCP/doctors are each enabled and motivated to contribute to the overall efficiency of the health care system of the example embodiment.

In particular, when used for managing diabetes patients, the system and method according to the example embodiment distinguish between diabetes type 1 patients and diabetes type 2 patients.

The following is a detailed description of an exemplary application present technology. Example 3: Improved Risk Detection and First Management for Cardiovascular Patients (Including Multimorbidity Patients with Diabetes Through the Comprehensive Empowerment by the present technology system).

Empowerment and a collaborative cardiovascular risk-patients as described above, the comprehensive BPPS approach, integrating:

Bio-Medical Factors (Bio-Marker),

Psychological Factors (Psycho-Marker),

Personal Characteristics and Traits (Perso-Marker), and

Socio-Economic Factors (Socio-Marker)

is combined through the NNS of the present technology with an activation of the patient with (the innovative embodiment of) the Three Step Model as shown in Table 6.

TABLE 6

| | |
|---|---|
| Step 1 | “Self-Assessment” of the Patient |
| Step 2 | “Reality Check” and (Lab Results) Feedback as well as |
| | Diagnosis of the |
| Step 3 | Physician/Medical Experts |
| | “Collaborative Care”: Patient and Physician cooperating |
| | in the use of the EEG (Electroencephalogram) and ECG |
| | (Electrocardiogramm) |

This is creating the (innovative and significantly improved) empowerment and preparation of the patient with cardiovascular risks for a significantly improved cardiovascular risk management, using the EEC and ECG measurement ranges with the NNS of Prof. Reuter as risk indicator.

The NNS of the present technology can be utilized for the classification of brain and cardiovascular conditions in risk management. This installation is an entitlement to the utilization of the procedure contained in and protected by patent DE 39 29 077 C2, the contents of which are incorporated herein by reference.

With the introduction of digital calculators that become ever smaller in size and faster it is now possible to measure, to process and to analyze in the online-modus highly complex and weak electromagnetic amplitudes. This offers the possibility to also include, to process and to analyze signals that are by their nature low-intensity biological or physiological signals of high relevance in cardiovascular therapy.

The simultaneous classification of brain and heart signals of low intensity can be accomplished by the present technology. Especially the simultaneous classification and identification of brain and heart signatures is an essential parameter to monitor the vital condition of individuals under changing and life-threatening circumstances.

Today it is the state of the art to apply a standardized measurement procedure of the pulse rate by analyzing its exact frequency over time with the electrocardiogram (ECG) and the brain activity through the electromagnetic activity of its neurons via electroencephalogram (EEG): This standardized procedure is applied to classify and to identity the ‘activity status’ of these two organs.

Simultaneous Medical Monitoring of Pulse Rate and Brain Activity

Especially with the more and more complex medically indicated interventions relating to vital cardiovascular functions or operations caused by traumata a simultaneous combined monitoring of these two vital parameters (brain and heart) is necessary. This is the only way to realize an effective classification and identification of the activity status of this vital combination: cardiovascular system and central nervous system (CNS).

This simultaneous monitoring combined with an equally simultaneous classification/identification of brain and heart status by their activity behavior of the innervating neurons is the object of the present patent application.

The reason for this is mainly the insufficient separation of low-intensity bio-signals from the noise that is masking the low-intensity bio-signals. The low-intensity bio-signals also can be separated from frequencies of other electromagnetically active organs and in case of the brain the ‘masking’ of the electromagnetic signals of the lower brain structures coordinating the vitality of the individual which is caused by the large tissue mass of the neocortex.

There is a failure of existing signal identification and monitoring methods. Because of these obstacles the existing signal processing, classification and identification procedures (that have been favored so far) fail an identification of low-intensity or ‘masked’ or screened activity centers is not possible or requires an enormous attention and routine of the respective applicant.

On the other hand many medical studies show that the pulse rate frequently is changing in the tenth or hundredth Hertz range while this pulse rate variability is equivalent to the permanent adjustment of the default offered by the brainstem. This makes the pulse rate range of minor variations the central parameter of the vitality of the cardiovascular monitoring system located in the brainstem, which is always active. When it extinguishes and/or sways without adequate correction in wide ranges, this is an indicator for severe damages and/or poisonings and/or malfunctions of the Central Nervous System (CNS), which can lead to the death of an individual.

Furthermore, the latest medical analyses show that a successfully combined standardization of EEC- and ECG-values cannot be expected in the future. They have to be considered rather as highly individual and dependent upon Lifestyle and bio-medical life history. The two parameters depend for example upon age, gender, physical and mental health, diabetes and other permanent diseases affecting the metabolism. Also climate and other not mentioned parameters might have an influence.

In addition, one can see that therapeutic actions can influence the ‘normal’ EEC- and ECG-values of an individual, which has consequences for the classification of the vital parameters.

The existing monitoring results are only a rough guideline. The values and results shown in the literature are therefore merely a rough guideline. In addition to a constant gathering of the vital parameters heart and brain activity modern ‘Analyzers’ have to be subject to conditioning—thus offering individual adjustability—if one is aiming at an adequate analysis of heart and brain activity.

Thus, conditioning for exact monitoring by NNS of the present technology can be accomplished. This conditioning can be realized by using neuronal networks, adaptive fuzzy-classificators or re-writeable media such as ROM, PROM, EPROM, EEPROM, in which the individual heart and brain activity pattern can be stored in form of learning patterns that are used as references for the current individual status.

All these guidelines have not been sufficiently solved by the existing ‘state of the art’ technology since the standard procedures separate data gathering and data analysis of EEC- and ECG-values; the values of the pulse rate are not analyzed in the tenth or hundredth Hertz range and no ‘micro changes’ of the individual heart and brain activity patterns are taken into consideration to classify the individual status.

The NNS-Classificator of the present technology can be utilized for cardio-vascular risk management. The example embodiment described here is covering and integrating all these focal aspects since it achieves and secures the simultaneous continuous and individual gathering of heart and brain activity data, analyzed in the tenth or hundredth Hertz range and establishes a very exact categorization and identification.

In an additional Example embodiment the present installation of the present technology system allows for a readjustment of the individual heart and brain activity patterns and allows defaults that can be altered. Therefore the present technology system is individualizing the evaluation criteria for the classification/identification of the activity status of the associations of neurons that are gathered and individualized. Additionally the present technology NNS-system is tracing the status alterations that have been produced by therapeutic measures, for example.

The following is an example embodiment of an medical risk management monitoring system in cardiovascular therapy of the present technology. All these tasks are for the first time ever solved by the characteristics of this patent claim in the presented way of the NNS system for monitoring of high risk cardiovascular and diabetes patients. Many modifications and variations of the example embodiment will be apparent to those of ordinary skill in the art in light of the foregoing disclosure. Therefore, it is to be understood that, within the scope of the appended claims, the example embodiment can be practiced otherwise than has been specifically shown and described.

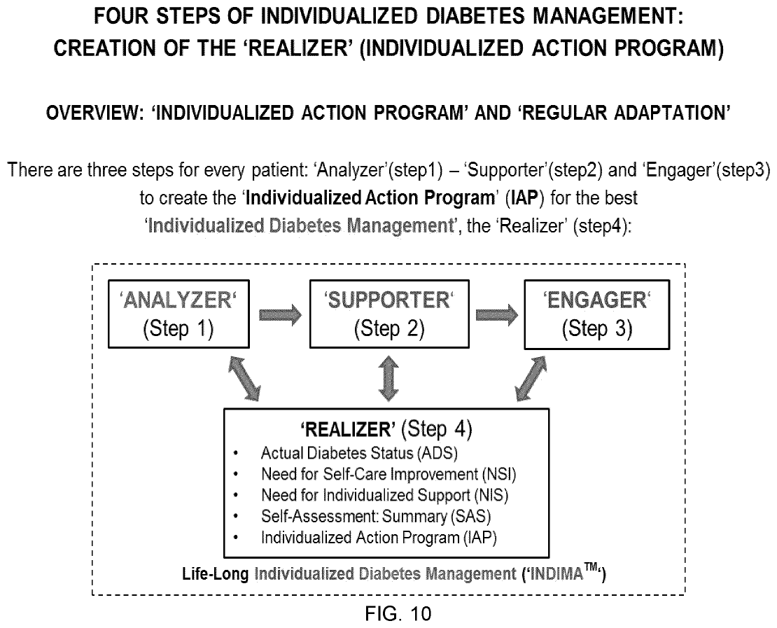

All of the systems, methods, and computer programs described above and in the claims can be applied and have been applied successfully to IHM or can be applied to any of the other five areas of application described in the introduction and in FIG. 10.

The program or method of the present technology can include the 10 question guide resulting in multiple reports for assisting the patient and/or the HCP team in understanding the need for action, options for action and risk management. Questions 1-4 can be related to self-assessment, while questions 5-10 can be related to the doctor or HCP team.

Additional data can be inputted into the present technology system or application software from peripheral devices, such as but not limited to, glucose monitoring systems and/or an activity monitoring system such as Actibelt®. Activity data can be utilized to create a physical activity profile. SQL-Based data reports can be created for physical and psychological state of the patient; blood glucose; cholesterol/lipids; and/or blood pressure for diabetes. Statistical processing with multiple correlation analysis and predictive models can also be created. The data can be transmitted to the present technology system via wired or wireless connection.

Using wearable activity monitoring device as an example, the movement data measured by device are correlated with the comprehensive patient profile, i.e. the diabetes patient's, which can include B-Bio-Medical, P-Psychological, P-Personal and S-Social-Systemic (socio-economic and cultural) data.

The resulting integration of the qualitative and quantitative mobility data with the BPPS-marker of the present technology shows all of the correlations, inter-dependencies and interrelations for an optimization of the Individualized Diabetes Management.

The monitoring device is empowering the diabetes patient, enabling to increased mobility, to independence and individualized support & training programs, improving the allover outcome significantly.

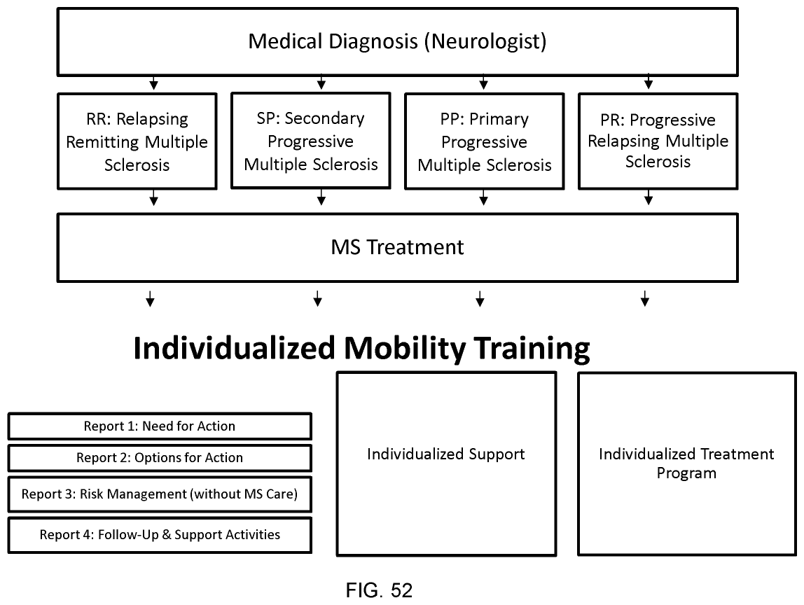

In the exemplary and as shown in FIG. 52, the present technology utilized with a peripheral activity monitoring device, can be configured or configurable for Individualized Multiple Sclerosis (IMS) management. The activity monitoring device can include a motion sensor (accelerometer) at body center of mass, and can be integrated in a belt. With the IHM of the present technology being a web-based system that can be integrated (Bluetooth/UMTS/LTE) to create an online monitoring system.

There are about 2.3 million patients worldwide diagnosed with Multiple Sclerosis (MS) (estimated by the National Multiple Sclerosis Society http://www.nationalmssociety.org/). The IHM of the present technology will provide the patient a chance to participate actively in their health management by empowering and enabling them to manage their Multiple Sclerosis actively. The IHM of the present technology can improve the physical and objective state of the patient, and at the same time giving a feeling of empowerment, enabling to do something about coping with Multiple Sclerosis.

The IHM of the present technology can be configured or configurable as a hybrid system with the activity monitoring device for continuous and personalized support in IMS management. It can provide self-direction & MS self-management plus measurement for feedback. This includes physical objective data+(BioMedical-, Psycho-, Perso-, Socio-Marker) creating the basis for the integrated monitoring device with the present technology.

It can be appreciated that there will be significant outcome improvement for MS patients. All existing MS medications will have significant effects through the combination of the medical treatment program with the present technology hybrid system, empowering and enabling the patient for Individualized Self-Care (ISC). This improvement can be achieved through:
(1) Individualized Self-Care and Mobility Training,
(2) Individualized Support Program, and
(3) Individualized MS Treatment Program.

Basis is the patient's self-direction and the ‘Individualized Self-Care’ which is triggered by the present technology hybrid system and the activity monitoring device measurement for feedback:

the activity monitoring device is measuring the physical objective data;

the IHM of the present technology is covering the M-P-P-S Marker (Medical-, Psycho-, Perso-, Socio-Marker);

the activity monitoring device and IHM of the present technology are integrated into the present technology hybrid system.

For the MS patient mobility and walking (autonomy and independence is of highest importance. The present technology is creating best possible individual support and training program. The present technology is a source of daily empowerment, leads to self-direction and is enabling the patient to experience quality of life with a relative maximum of autonomy and independence.

Thus, there is a paradigm shift in Multiple Sclerosis Management through the present technology hybrid system, leading to an optimized self-care and an individualized mobility training, resulting in a significant 10%-50% or even more outcome improvement.

The movement data, measured by activity monitoring device, are correlated with the comprehensive patient profile, i.e. the MS patient's B=Biomedical, P=Psychological, P=Personality, and S=Social (socio-economic and cultural) data.

The resulting integration of the qualitative and quantitative mobility data (movement data) with the BPPS-marker of the present technology system shows all of the correlations, inter-dependencies and interrelations for an optimization of the ‘Individualized MS Management’.

The present technology hybrid system is empowering the MS patient, enabling to increased mobility, to independence and ‘Individualized Support & Training Programs’, improving the all over outcome significantly.

Improvement of Individualized MS Management through patient self-analysis, partner and social environment support and individualized treatment program based upon ‘monitoring and mobility training associated with the present technology hybrid system.

The social, personality, psychological and empirical mobility data for an individualized training and support program and an optimized MS management can be combined.

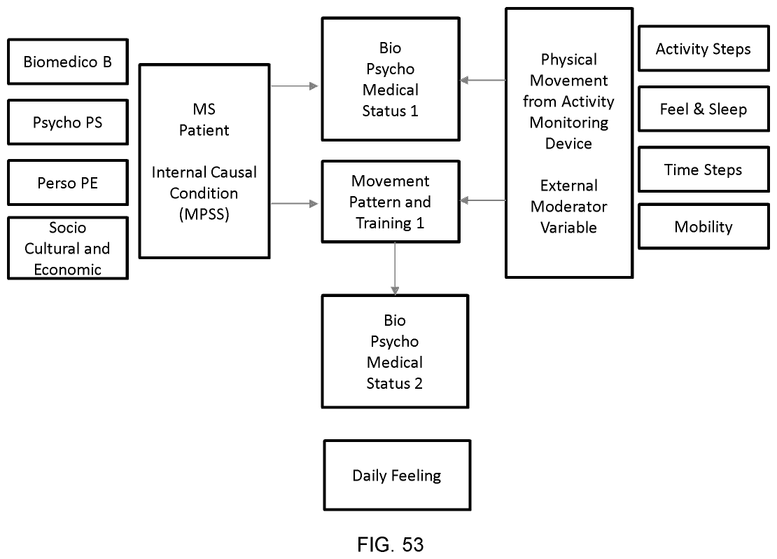

Using the patient's risk profile predicted by the activity monitoring device a present technology based 4-factor preventive optimization program (MPPS): Medico, Psycho, Perso, and Socio elements can be provided, as shown in FIG. 53.

The program or method can further include a further report for priorities of actions. This could be customized or prioritized for the patient, the family group, and the HCP team.

In addition to the above, embodiment of the present technology can be configured or configurable so that previous user input expands to direct communication, such as but not limited to a chat box, video conferencing with a professional in area of concern for the user or based on the analysis of results measured as indicated by previous system.

Previous user input can expand to direct neural and coronary artery input or connection.

Previous user input can expand to environment management analysis such as, but not limited to, immune system analysis, hygiene analysis and antibiotic resistance analysis in light of present circumstances.

Previous user input can expand to life management analysis inclusive of financial, social interaction, physical activity and vocation development.

In various example embodiments, an electronic device can utilize the present technology and can operate as a standalone device or may be connected (e.g., networked) to other devices. In a networked deployment, the electronic device may operate in the capacity of a server or a client machine in a server-client network environment, or as a peer machine in a peer-to-peer (or distributed) network environment. The electronic device may be a personal computer (PC), a tablet PC, a set-top box (STB), a personal digital assistant (PDA), a cellular telephone, a web appliance, a network router, switch or bridge, or any machine capable of executing a set of instructions (sequential or otherwise) that specify actions to be taken by that device. Further, while only a single electronic device is illustrated, the term “device” shall also be taken to include any collection of devices that individually or jointly execute a set (or multiple sets) of instructions to perform any one or more of the methodologies discussed herein.

The processor unit and memory of the electronic device can communicate with each other via a bus. In other embodiments, the electronic device may further include a video display (e.g., a liquid crystal display (LCD)). The electronic device may also include an alpha-numeric input device(s) (e.g., a keyboard), a cursor control device (e.g., a mouse), a voice recognition or biometric verification unit (not shown), a drive unit (also referred to as disk drive unit), a signal generation device (e.g., a speaker), a universal serial bus (USB) and/or other peripheral connection, and a network interface device. In other embodiments, the electronic device may further include a data encryption module (not shown) to encrypt data.

The processing unit can be a module operably associated with a drive unit, with the drive unit including a computer or machine-readable medium on which is stored one or more sets of instructions and data structures (e.g., instructions) embodying or utilizing any one or more of the methodologies or functions described herein. The instructions may also reside, completely or at least partially, within the memory and/or within the processors during execution thereof by the electronic device. The memory and the processors may also constitute machine-readable media.

The instructions may further be transmitted or received over a network via the network interface device utilizing any one of a number of well-known transfer protocols (e.g., Extensible Markup Language (XML)). While the machine-readable medium is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the device and that causes the device to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

It can be appreciated that the present technology integrates:

(1) the Self-Care responsibility of the Patient (Client),

(2) the unconditioned acceptance of the Patient (Client) by the Doctor and his team, and

(3) the cost efficient best quality with the ‘Individualized Treatment Program’ and the ‘Individualized Support Program’ for the Patient (Client), Partner, Family and Diabetes Group:

It can be further appreciated that the present technology integrates provides a best quality and responsible cost-efficiency through a 360 degree approach. The Synergy Patient (PSA), Doctor and Diabetes Team (MDT=Medical Team) and the social environment (PFG) leads to best possible

### Individualized Self-Care

### Individualized Support Program

### Individualized Treatment Program.

The IHM of the present technology can include further development of its comprehensive approach “for all 10 patient groups on three levels I, II, III or in three 3 steps:
Step 1/Level I: PHR=Personalized Health Report for 10 groups
   page 1: Status=Diagnosis, Treatment, Medication
   page 2: IAP=Individualized Action Program
   ‘OnePager’=Individualized Action Plan
Step 2/Level II: Individualized Disease Management=guide system of the present technology (5 App-Instruments for each one of the 10 groups)
   1 and 2: diabetes type 1 and type 2;
   3-8 Individualized Disease Management for 6 additional chronic diseases (Groups 3-8)
   for normal patients with Acute Disease Management (ADM) (Group 9)
   and for Handicapped Health Care (HHC) (Group 10)
10 Groups with 5 App-Instruments each=50 App-Instruments for Guide Systems for all patients

Step 3/Level III: 4 Step Collaborative Care (SIP Group development)
as innovation and complete new development of the Guide System within SIP Group (master version):
   support of Roche Diabetes Care;
   result: a Diabetes Care Guide, consisting of 8 reports
   very positive results of Focus Studies in Portugal (Lisbon and Porto) and respective design of a specific Portuguese version of Diabetes Guide of the present technology;
   very positive results of application studies in Medical Centers

It can be appreciated that the 10 stable success factors of the present technology, as discussed above, can be utilized for other diseases, for normal patients and for handicapped and for health management & prevention; although the loadings of the 10 success factors were differing among the among the 10 Groups differentiated by the IHM system of the present technology.

In the exemplary, this can be utilized in 8 groups of chronic diseases comprised in the IHM system. These exemplary groups can be:
Group 1—Diabetes Type 1
Group 2—Diabetes Type 2
Group 3—Cardiovascular Diseases
Group 4—Oncology
Group 5—Multiple Sclerosis
Group 6—Pain Patients
Group 7—Respiration and Allergy Patients
Group 8—Psychosomatic Patients
Group 9—the so-called normal and basically healthy persons/patients without a chronic disease (1-8) and without a handicap or being disabled (group 10) and
Group 10—the handicapped persons/patients

In a further exemplary, a patient with an eating disorder (which is conditioned through all your life) can utilize the present technology and needs in the first step unconditioned acceptance (and not critic, since you are okay), empowerment and enabling.

The second step is cooperation with the diabetes and control of your diabetes. The third and final step is adaptation and coping, leading to healthy living and quality of life. This will lead to the best possible diabetes management.

While embodiments of the individualized and collaborative health care system, method and computer program have been described in detail, it should be apparent that modifications and variations thereto are possible, all of which fall within the true spirit and scope of the present technology. With respect to the above description then, it is to be realized that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology. For example, any suitable sturdy material may be used instead of the above-described. And although diabetes management has been described, it should be appreciated that the individualized and collaborative health care system, method and computer program herein described is also suitable for the management of any chronic disease or health issued, and can further be implementable for financial management.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A system for individualized and collaborative health care using groups of state parameters for defining a state of each individual, and using groups of action parameters for defining treatment options, support options and/or behavior options targeted at an individual within said plurality of individuals, the system comprising:
at least one sensor configured to ascertain physiological or psychological sensor data of the targeted individual; and
a computer system in communication with the sensor, the computer system comprises one or more processors programmed with computer program instructions which, when executed cause the computer system to:
convert the sensor data to a sensor data vector in a defined sequence;
process input data that is based on the groups of state parameters and the sensor data vector, into output data, which are the basis for the groups of action parameters, using defined relationships/assignments between groups of state parameters and groups of action parameters;
process one or more estimators based on the sensor data vector in a hierarchical manner;
store, on at least one data storage device, the groups of state parameters, the groups of action parameters and the defined relationships/assignments between groups of state parameters and groups of action parameters;
define at least one state of each of the individuals using the output data, the state of the individuals being in part defined from a social module, a personal module and a psychological module that are implemented by the computer system;
receive medical information about the individuals;
compare the state of the individuals and the medical information by determining a deviation from at least part of the state of the individuals and at least part of the medical information;
define at least one treatment or behavior option using the groups of action parameters, the action parameters being defined in part from the social module, the personal module, the psychological module, and the deviation;
target the treatment or behavior option to a targeted individual within the plurality of individuals;
generate a predicted state of health of the targeted individual at a pre-determined time period utilizing a neural chain of the estimators, and classifying the targeted individual to a category of a plurality of categories according to the predicted state, and providing the predicted state of health with the treatment or behavior option; and
communicate to the targeted individual, by way of a data communication system, the treatment or behavior option, state parameters selected from the groups of state parameters and/or action parameters selected from the groups of action parameters among the plurality of individuals;
a graphical user interface operably implemented or implementable on the computer system and executable by the processors.

2. The system according to claim 1, wherein the graphical user interface being configured or configurable to initiate direct communication between the targeted individual and a health care professional.

3. The system according to claim 2, wherein the direct communication is video chat utilizing a camera in operable communication with the processor.

4. The system according to claim 1, wherein the state parameter group is based on observation, evaluation and assessment of the health care client using a web-based questionnaire sent to the targeted individual by way of a communication interface of the computer system.

5. The system according to claim 4, wherein the web-based questionnaire is configured or configurable to provide information regarding self-assessments of a medical and physiological condition of the targeted individual, information regarding a psychological condition of the targeted individual, information regarding a personality trait, communication style, genetic factors, and/or behavior patterns of the targeted individual, and information regarding fitness, activities, and/or lifestyle of the targeted individual.

6. The system according to claim 5, wherein the information from the web-based questionnaire is used in part by the processor to define at least one parameter in the group of state parameters by assigning a marker or value for the targeted individual, and wherein the client-specific action parameter group is created by the processor where each parameter in the client-specific action parameter group is assigned a marker or value for the targeted individual.

7. The system according to claim 5, wherein the treatment or behavior option includes at least one report selected from the group consisting of rating the targeted individual condition associated with groups of success factors relating to at least one question in the web-based questionnaire, supporting further detailed self-assessment of the targeted individual, and categorizing an action to be conducted by the targeted individual.

8. The system according to claim 1, wherein the treatment or behavior option further includes need-for-action levels selected from the group consisting of a first level where the deviation is determined to be at a first predetermined value, a second level where the deviation is determined to be at a second predetermined value that is less than the first predetermined value, a third level where the deviation is determined to be a third predetermined value that is less than the second predetermined value, and a fourth level where no deviation is found.

9. The system according to claim 1, wherein the computer system further includes a data interface for data acquisition, the data interface is configured or configurable to receive biomedical information selected from the group consisting of blood pressure, lipids, and blood glucose level.

10. The system according to claim 1, wherein the defined relationships/assignments between groups are redefined/updated using empirical pairs/empirically defined relations and neural networks determined relations of action parameter groups and state parameter groups, and wherein the neural networks comprises a self-organizing map constructed from a set of the action parameters, a set of predetermined action levels, and corresponding predetermined disease progression data.

11. The system according to claim 1, wherein the estimators are coded to be placed on a topologically closed, two-dimensional surface on a regular or irregular grid formed of the estimators configured to assign a same number of adjacent estimators to every the estimator.

12. A method for individualized and collaborative health care involving a plurality of individuals, using groups of state parameters that define a state of each individual, and using groups of action parameters that define individualized treatment options, individualized support options and/or individualized behavior options targeted at a targeted individual within the plurality of individuals, the method being implemented in a computer system that includes one or more physical processors configured to execute one or more computer program modules, the method comprising the steps of:
ascertaining physiological or psycho-medical sensor data of the targeted individual utilizing at least one sensor;
converting, using the processors, the sensor data to a sensor data vector in a defined sequence;
processing, using the processors of the computer system, input data received by the computer system and the sensor data vector, which are based on the groups of state parameters, into output data, which are the basis for the groups of action parameters, using defined relationships/assignments between groups of state parameters and groups of action parameters;
storing, on at least one data storage device of the computer system, the groups of state parameters, the groups of action parameters and the defined relationships/assignments between groups of state parameters and groups of action parameters;
defining, using the processors of the computer system, at least one state of each of the individuals using the output data, the state of the individuals being in part defined from a social module, a personal module and a psychological module;
processing, using the processors of the computer system, medical information associated with the individuals;
comparing, using the processors, the state of the individuals and the medical information by determining a deviation from at least part of the state of the individuals and at least part of the medical information;
defining, using the processors of the computer system, at least one treatment or behavior option or an individualized action program using the groups of action parameters, the action parameters being defined in part from the social module, the personal module, the psychological module, and the deviation;
processing, using the processors of the computer system, one or more estimators based on the sensor data vector in a hierarchical manner;
generating a predicted state of health of the targeted individual at a pre-determined time period utilizing a neural chain of the estimators, and classifying the targeted individual to a category of a plurality of categories according to said predicted state, and providing the predicted state of health with the treatment or behavior option or the individualized action program;
communicating to the targeted individual the treatment or behavior option using a communication interface of the computer system, state parameters selected from the groups of state parameters and/or action parameters selected from the groups of action parameters among the plurality of individuals; and
initiating direct communication between the targeted individual and a health care professional by way of a graphical user interface operably implemented or implementable on the computer system and executable by the processors.

13. The system according to claim 12, wherein the direct communication is video chat utilizing a camera in operable communication with the processor.

14. The method according to claim 12, wherein a health care client-specific the state parameter group is determined by assessing the health care client using a web-based questionnaire, and wherein the web-based questionnaire is configured or configurable to provide information regarding self-assessments of a medical and physiological condition of the targeted individual, information regarding a psychological condition of the targeted individual, information regarding a personality trait, communication style, genetic factors, and/or behavior patterns of the targeted individual, and information regarding fitness, activities, and/or lifestyle of the targeted individual.

15. The method according to claim 14, wherein the treatment or behavior option or the individualized action program is at least in part dependent on the information provided by the web-based questionnaire.

16. The method according to claim 14 further comprising the steps of:
defining at least one parameter in the group of state parameters by in part using the information from the web-based questionnaire to assign a marker or value for the targeted individual; and
creating the client-specific action parameter group where each parameter in the client-specific action parameter group is assigned a marker or value for the targeted individual.

17. The method according to claim 14 further comprises the step of creating at least one report and associating the report with the treatment or behavior option, the report being selected from the group consisting of rating the targeted individual condition associated with groups of success factors relating to at least one question in the web-based questionnaire, supporting further detailed self-assessment of the targeted individual, and categorizing an action to be conducted by the targeted individual.

18. The method according to claim 12 further comprises the step of receiving biomedical information using a data interface of the computer system, the data interface is configured or configurable for data acquisition, the biomedical information being selected from the group consisting of blood pressure, lipids, and blood glucose level, and wherein the treatment or behavior option is at least in part dependent on the biomedical information.

19. The method according to claim 7, wherein the defined relationships/assignments between groups are redefined/updated using empirical pairs/empirically defined relations and neural networks determined relations of action parameter groups and state parameter groups, and wherein the neural networks comprises a learning system based upon a self-organizing map constructed from a set of the action parameters, a set of predetermined action levels, and corresponding predetermined disease progression data.

20. A non-transitory computer readable medium with an executable program stored thereon comprising instructions for execution by at least one processing unit for individualized and collaborative health care involving a plurality of individuals, using groups of state parameters that define a state of each individual, and using groups of action parameters that define individualized treatment options, individualized support options and/or individualized behavior options targeted at a targeted individual within the plurality of individuals, such that the instructions when executed by the at least one processing unit cause the at least one processing unit to:
ascertain physiological or psycho-medical sensor data of the targeted individual utilizing at least one sensor;
convert, using the processors, the sensor data to a sensor data vector in a defined sequence;
process, using the processors of the computer system, input data received by the computer system and the sensor data vector, which are based on the groups of state parameters, into output data, which are the basis for the groups of action parameters, using defined relationships/assignments between groups of state parameters and groups of action parameters;
store, on at least one data storage device of the computer system, the groups of state parameters, the groups of action parameters and the defined relationships/assignments between groups of state parameters and groups of action parameters;
define, using the processors of the computer system, at least one state of each of the individuals using the output data, the state of the individuals being in part defined from a social module, a personal module and a psychological module;
process, using the processors of the computer system, medical information associated with the individuals;
compare, using the processors, the state of the individuals and the medical information by determining a deviation from at least part of the state of the individuals and at least part of the medical information;
define, using the processors of the computer system, at least one treatment or behavior option or an individualized action program using the groups of action parameters, the action parameters being defined in part from the social module, the personal module, the psychological module, and the deviation;
process, using the processors of the computer system, one or more estimators based on the sensor data vector in a hierarchical manner;
generate a predicted state of health of the targeted individual at a pre-determined time period utilizing a neural chain of the estimators, and classifying the targeted individual to a category of a plurality of categories according to the predicted state, and providing the predicted state of health with the treatment or behavior option or the individualized action program;
communicate to the targeted individual the treatment or behavior option using a communication interface of the computer system, state parameters selected from the groups of state parameters and/or action parameters selected from the groups of action parameters among the plurality of individuals; and
initiate direct communication between the targeted individual and a health care professional by way of a graphical user interface operably implemented or implementable on the computer system and executable by the processors.
